(19) United States
(12) Patent Application Publication
(10) Pub. No.: US 2022/0119549 A1
(43) Pub. Date: Apr. 21, 2022

(54) SHIELDED BIOLOGICS WITH MASKING DOMAINS TO SHIELD ANTIGEN BINDING CAPABILITY OF BIOLOGICS AND USES THEREOF

(71) Applicant: Tavotek Biotherapeutics (Hong Kong) Limited, Shung Wan (HK)

(72) Inventors: Di ZHANG, Hillsborough, NJ (US); Lihua SHI, Wynnewood, PA (US); Minseon CHO, Breinigsville, PA (US); Motohiko NISHIDA, Ambler, PA (US); Man-Cheong FUNG, Ringoes, NJ (US); Mark CHIU, Paoli, PA (US)

(73) Assignee: Tavotek Biotherapeutics (Hong Kong) Limited

(21) Appl. No.: 17/501,036

(22) Filed: Oct. 14, 2021

Related U.S. Application Data
(60) Provisional application No. 63/092,061, filed on Oct. 15, 2020.

(51) Int. Cl. C07K 16/32; C12N 15/63; C07K 16/24; A61P 35/00

(52) U.S. Cl. CPC C07K 16/32; C12N 15/63; C07K 16/241; A61K 2039/507; C07K 2317/565; C07K 2317/31; C07K 2319/03; A61P 35/00

(57) ABSTRACT

The present disclosure relates to a shielded biologic with a masking domain, e.g., based on Insulin-like Growth Factor 2 (IGF2), fused to the Fab domain of an antibody via protease-cleavable linker to mask the antigen binding capability of the antibody, and to the use of said shielded biologic for effective and site-specific disease treatment while reducing systematic toxicity.

### CROSS-REFERENCE TO RELATED APPLICATION

This application claims the priority of Provisional Application No. 63/092,061, filed on Oct. 15, 2020, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 11, 2021, is named 15271_0004-00000_SL.txt and is 215,340 bytes in size.

### FIELD OF THE DISCLOSURE

The present disclosure relates to shielded biologics, e.g., using a TavoPRECISE-Shield technology, to engineer biologics with novel IGF2-based masking domains to shield their antigen binding capability while the shielded biologics can be selectively activated in a desired diseased tissue for therapeutic treatment.

### BACKGROUND OF THE DISCLOSURE

Therapeutic biologic drugs, more specifically, monoclonal antibodies and Fc fusion proteins, have become popular in the treatment of cancer, immunological diseases, infectious diseases, and metabolic diseases. In contrast to small molecule chemical compounds, antibody drugs have the advantage of having higher target selectivity, for better safety and more favorable pharmacokinetic profiles. Since the commercialization of the first antibody drug Muromonab in 1986 for transplant rejection, the value of the biologics market has grown tremendously and continues to grow at a fast pace in the biopharmaceutical market. To date there have been 100 antibody drugs approved for clinical use and hundreds more being developed for various diseases (Mullard 2021).

Although having great success along with huge clinical and commercial potential, there are still many challenges to overcome in order to develop more efficacious and safer antibody-based biologic drugs for patients. One of these challenges is mechanism-based on-target, off-site toxicities associated with systemic administration of antibody drugs to a target which happens to have important physiological roles in normal tissues besides pathological roles in disease states. For example, tumor necrosis factor alpha (TNFα) is a pathological proinflammation factor which is critical in the development of many autoimmune diseases. In fact, anti-TNFα antibodies have become major therapies for rheumatoid arthritis (RA), Crohn's disease and many other types of autoimmune diseases. As a matter of fact, anti-TNFα biologics drugs, including Humira, Remicade, Embrel and Simponi, constitute the most successful drug class in the biopharmaceutical marketing history. However, physiologically TNFα is an immune modulator critical for host defense, and blockade of TNFα with anti-TNFα biologics drugs may lead to serious on-target toxicities including severe infections and elevated risk of malignancy (Fiorentino, Ho et al. 2017, Fernandez-Ruiz and Aguado 2018). Therefore, chronic use of anti-TNFα biologics drugs pose a great risk to patients. On the other hand, anti-tumor antibody drugs targeting HER2 (e.g., Trastuzumab) and VEGF (e.g., Bevacizumab) are popular drugs for the treatment of many solid tumors. However, due to the presence of these drug targets, as well as their physiological roles in some normal tissues including the heart and the vasculature, usage of anti-HER2 and anti-VEGF therapeutic antibodies can pose serious adverse effects including heart failure and other cardiac dysfunctions in some patients (Touyz, Herrmann et al. 2018, Agunbiade, Zaghlol et al. 2019). Such mechanism-of-action based toxicities greatly limit the choice of these therapeutic antibodies as treatment options for some patients and/or limit the therapeutic window in pushing a higher dose to maximize potential efficacy.

As another example, immune checkpoint inhibitors, such as antibodies to PD-1, PD-L1, and CTLA-4, have shown outstanding efficacy in the treatment of many types of cancers and transformed the therapeutic landscape of oncology. However, severe immune-related adverse events, including rash, diarrhea, colitis, hypophysitis, hepatotoxicity and hypothyroidism, are often associated with some of these immune-oncology drug treatments (Spiers, Coupe et al. 2019). In many cases such severe toxicities can be life-threatening and limit duration of the treatment. Adverse effects have been reported more frequently with CTLA-4 inhibitors than PD-1/PD-L1 inhibitors, with the worst case being for bispecific antibody to PD-1 and CTLA-4. Such mechanism-based toxicities are due to the non-discriminated attack of normal tissues by the activated immune cells associated with the systemic treatments, especially for CTLA-4 which may play more important physiological roles in many different organs. Besides immune checkpoint inhibitors, anti-CD47 antibody drugs have shown great promise as a novel anti-tumor treatment in recent years by enhancing phagocytosis of tumor cells. However, some patients with the anti-CD47 drug treatment had anemia due to the significant expression of CD47 in different types of blood cells (Zhang, Huang et al. 2020).

T cell redirection therapies, including bispecific T cell engagers and CAR-T treatments, also showed significant therapeutic efficacies, but in the meantime posed severe risks for potential life-threatening adverse effects in some patients due to the dysregulated activated T cells attacking the same targets in normal tissues besides those in the tumor. Another promising anti-tumor treatment in recent years is antibody drug conjugates (ADC) which enable the delivery of cytotoxic drug payloads to tumor cells by antibodies targeting tumor antigens. However, such treatment may suffer severe side effects if the antibody part on the ADC recognizes the same target expressed in healthy tissues and inadvertently directs the cytotoxic payloads to kill the normal cells.

To overcome these safety concerns and thereby broadening the therapeutic index, it is critical to improve the site of action of these therapeutic antibodies by specific engagement of targets, preferably at the disease site, while sparing the same targets expressed in normal healthy tissue. One promising strategy is to add a protease-cleavable masking domain to mask the antigen binding domain of biologics as a shielded biologic (Lin, Lu et al. 2020). With the antigen binding capability masked by the masking domain, the engineered shielded biologics may bypass and not bind to bystander targets in otherwise normal tissues. Meanwhile, deregulation of various proteases is involved in the pathogenesis of many cancers, autoimmune diseases and inflammatory disorders. Such over-abundant disease-specific proteases may locally cleave off the masking domain from the shielded biologics, and thus unlock and turn on the activity of therapeutic antibody selectively at the disease site. By using a shielded antibody, the selectivity of antibodies to targets in the disease region, rather than healthy tissues, is greatly improved which translates into higher therapeutic index and better safety profile.

Several different masking strategies have been developed in recent years for antibody prodrug development (Kavanaugh 2020, Lin, Lu et al. 2020). These masking strategies can be broadly categorized into two types. The first type is a spatial hindrance-based approach which relies on the fusion of a masking domain to sterically interfere with the antigen-binding capability of the antibody. Certain antibody domains have been developed by AbbVie and Roche to sterically hinder the antibody binding to its target. Besides, autologous IgG hinge domain, coiled-coil domain, non-antibody protein fragment, such as latency-associated peptide, and amino acid polymer have also been developed as such masking domains (Metz, Panke et al. 2012, Chen, Chuang et al. 2017, Lu, Chuang et al. 2019, Trang, Zhang et al. 2019). Another type of strategies is affinity peptide-based which relies on affinity peptide or mutated antigen to occupy the antigen-binding, and thus exclude the antibody binding to its target antigen (Donaldson, Kari et al. 2009, Autio, Boni et al. 2020). For example, affinity peptides are used to mask the antibody as Probody developed by Cytomx, and Akrevia and Harpoon have similar strategies to mask the antibody known as Xilio and ProTriTac respectively.

All these different masking strategies have various pros and cons in terms of the size of the masking domain, masking and unmasking efficiency, and immunogenicity and universal applicability. In view of this, there remains additional need to develop novel masking methods with improved features including better masking efficacy to minimize systematic toxicity, better unmasking to increase drug efficacy in the disease site, specific unmasking with disease-specific protease-cleavable linker, better choice of sequences to minimize neo-epitopes, and/or better antibody engineering for optimized PK and/or drug stability in disease sites. The goal is to equip therapeutic antibodies with enhanced efficacy and safety to achieve a wider therapeutic index to allow potentially higher drug dosing for optimal efficacy with less safety concerns. This disclosure addresses safety along with providing novel approaches to improve biologic potency and preciseness.

### SUMMARY OF THE DISCLOSURE

The disclosure provides a shielded biologic comprising a heavy chain polypeptide comprising, from N-terminus to C-terminus, a first masking domain unit, a first protease-cleavable linker and an antibody heavy chain or an antigen-binding fragment thereof; and a light chain polypeptide comprising, from N-terminus to C-terminus, a second masking domain unit, a second protease-cleavable linker and an antibody light chain or an antigen-binding fragment thereof, wherein the first masking domain unit and the second masking domain unit form a masking domain. The masking domain is capable of shielding the antigen binding capability of the biologic in normal tissues, and in target disease tissues, one or both of the masking domain units are cleaved off by one or more disease site-specific proteases and the shielded biologic is converted to an active biologics.

In some embodiments, the disclosure provides a novel antibody engineering approach to make a shielded biologic by TavoPRECISE-Shield comprising a masking domain, e.g., wherein the A-chain and B-chain of Insulin Growth Factor 2 (IGF2) is fused to the N-terminus of antibody variable domains via a protease-cleavable linker sequence, respectively. In some embodiments, the IGF2-containing masking domain shields the antigen-binding capability of the biologic by steric hindrance in normal tissues. In disease tissues, the overexpressed proteases can cleave off the masking domain, so the shielded biologic by TavoPRECISE-Shield is converted to an active biologic which allows for full functional activity to its target (e.g., illustrated by FIG. 1). This IGF2-based shielded biologics by TavoPRECISE-Shield approach can facilitate an enhanced site-of-action selectivity of the antibody by acting on the targets in disease tissues while sparing targets in normal tissues, thus enhancing the safety profile of the biologics by reducing systemic toxicity.

In some embodiments, the disclosure provides an IGF2-containing masking domain comprising IGF2 A chain and B chain with one or more mutations that disrupt IGF2 binding to its receptor. In some embodiments, the disclosure provides a IGF2-containing masking domain comprising IGF2 A chain with a V43L mutation and IGF2 B chain with a Y27A mutation that disrupt IGF2 binding to its receptor.

As non-limiting examples, the disclosure provides shielded biologics by TavoPRECISE-Shield in which the A-chain and B-chain of IGF2 are fused, via a protease-cleavable linker sequence, to the N-terminus of heavy chains and light chains, respectively, of therapeutic antibodies against one or multiple drug targets, including TNFα, IL-1β, HER2, VEGF, EGFR, cMET, Nectin-4, CTLA-4, CD3ε, a4-integrin, CD20, CD11a, CD52, RANK-L, PD-1, PD-L1, CD47, CD24, CD166, and CD71.

The shielded biologics by TavoPRECISE-Shield with a masking domain (e.g., IGF2-based) provided in this disclosure can be monoclonal antibodies, bispecific or multi-specific antibodies, fusion proteins, antibody drug conjugates (ADC), or chimeric antigen receptor (CAR)s. The shielded biologics by TavoPRECISE-Shield can be full length IgG, IgA, IgD, IgM antibodies or may comprise only an antigen-binding portion including a F_{ab}, F(_{ab′})₂, scFv fragment or single chain domain VHH. The backbones of the shielded biologics by TavoPRECISE-Shield may be modified to affect functionality, e.g., to reduce or eliminate residual effector functions.

The disclosure also provides a shielded biologic by TavoPRECISE-Shield with a masking domain (e.g., IGF2-based) with an extended half-life when compared to the native antibody. The extension of half-life can be realized by engineering the C_{H2} and C_{H3} domains of the antibody with any one or more sets of mutations selected from M252Y/S254T/T256E, M428L/N434S, T250Q/M428L, N434A and T307A/E380A/N434A when compared to a parental native antibody, residue numbering according to the EU Index.

The disclosure also provides a shielded biologic by TavoPRECISE-Shield with a masking domain (e.g., IGF2-based) with enhanced resistance to proteolytic degradation by a protease that cleaves the native antibody between or at residues 222-237 (EU numbering). The resistance to proteolytic degradation can be realized by engineering E233P/L234A/L235A mutations in the hinge region with G236 deleted in the shielded biologic when compared to a parental wild-type antibody, residue numbering according to the EU Index.

The disclosure also provides a polynucleotide encoding a shielded biologic by TavoPRECISE-Shield in which a masking domain (e.g., IGF2-based) is fused to the N-terminus of antibody heavy chain and light chain as disclosed herein.

The disclosure also provides a vector comprising one or more polynucleotides of the disclosure.

The disclosure also provides a host cell comprising one or more vectors of the disclosure.

The disclosure also provides a method of generating a shielded biologic by TavoPRECISE-Shield comprised with a masking domain (e.g., IGF2-based) fused to the antibody variable domains via a protease-cleavable linker, comprising culturing a host cell of the disclosure under conditions that the shielded biologic by TavoPRECISE-Shield is expressed, and purifying the shielded biologic by TavoPRECISE-Shield.

The disclosure also provides a pharmaceutical composition comprising a shielded biologic by TavoPRECISE-Shield with a masking domain (e.g., IGF2-based) of the disclosure and a pharmaceutically acceptable carrier.

The disclosure also provides a method of cleaving the masking domain (e.g., IGF2-based) off of a shielded biologic by TavoPRECISE-Shield disclosed herein by different types of protease, thus converting the shielded biologic by TavoPRECISE-Shield into an active antibody.

The disclosure also provides a method of detecting cleavage of a masking domain (e.g., IGF2-based) from a shielded biologic by TavoPRECISE-Shield disclosed herein by different types of proteases.

The disclosure also provides a method of measuring the antigen-binding capability of a shielded biologic by TavoPRECISE-Shield with a masking domain (e.g., IGF2-based) and the active biologic after the masking domain is cleaved off by proteases.

The disclosure also provides a method of measuring the functional activities of a shielded biologic by TavoPRECISE-Shield with a masking domain (e.g., IGF2-based) and the active biologic after the masking domain is cleaved off by a protease by in vitro assays.

The disclosure also provides a method of measuring the in vivo efficacy of a shielded biologic by TavoPRECISE-Shield with a masking domain (e.g., IGF2-based) and the active biologic after the masking domain is cleaved off by a protease in animal drug potency studies.

The disclosure also provides a method of measuring the safety profile of a shielded biologic by TavoPRECISE-Shield with a masking domain (e.g., IGF2-based) and the active biologic after the masking domain is cleaved off by a protease in animal toxicity models.

The disclosure also provides a method of measuring the half-life of a shielded biologic by TavoPRECISE-Shield with a masking domain (e.g., IGF2-based) and the active biologic after the masking domain is cleaved off by a protease.

The disclosure also provides a method of treating an auto-immune/inflammatory disease in a subject, comprising administering to the subject a therapeutically effective amount of the shielded biologic by TavoPRECISE-Shield with a masking domain (e.g., IGF2-based). The disclosure also provides use of a shielded biologic by TavoPRECISE-Shield with a masking domain (e.g., IGF2-based) provided herein in a method of treating an auto-immune/inflammatory disease; and for use of a shielded biologic by TavoPRECISE-Shield provided herein in the manufacture of a medicament for use in an auto-immune/inflammatory disease. Exemplary auto-immune and/or inflammatory diseases include, but are not limited to, the following: rheumatoid arthritis, systemic lupus erythematosus, osteoarthritis, ankylosing spondylitis, Behcet's Disease, gout, psoriatic arthritis, multiple sclerosis, Crohn's colitis, and inflammatory bowel disease.

The disclosure also provides a method of treating an inflammatory or autoimmune disease condition such as Type I and II diabetes, as well as other inflammatory neuro, ophthalmic and dermatologic conditions in a subject, comprising administering to the subject a therapeutically effective amount of a shielded biologic by TavoPRECISE-Shield with a masking domain (e.g., IGF2-based). The disclosure also provides use of a shielded biologic by TavoPRECISE-Shield with an IGF2-based masking domain provided herein in a method of treating a disease such as diabetes, nerve, eye, and skin diseases; and for use of a shielded biologic by TavoPRECISE-Shield provided herein in the manufacture of a medicament for use in a disease such as diabetes, nerve, eye, and skin diseases. Exemplary diseases include but are not limited to: Type II diabetes mellitus, Parkinson's disease, age-related macular degeneration (AMD), polyneuropathy, sensory peripheral neuropathy, proliferative diabetic retinopathy, diabetic neuropathy, decubitus ulcer, fulminant Type 1 diabetes, retinal vasculitis, non-infectious posterior uveitis, and alcoholic neuropathy.

The disclosure also provides a method of treating cancer in a subject, comprising administering to the subject a therapeutically effective amount of a shielded biologic by TavoPRECISE-Shield with a masking domain (e.g., IGF2-based). The disclosure also provides use of the shielded biologic by TavoPRECISE-Shield with the IGF2-based masking domain provided herein in a method of treating cancer; and for use of a shielded biologic by TavoPRECISE-Shield provided herein in the manufacture of a medicament for use in cancer. Exemplary cancers include, but are not limited to: multiple myeloma, non-small cell lung cancer, acute myeloid leukemia, breast cancer, pancreatic cancer, bile duct cancer, colorectal cancer, esophageal/GE junction cancer, bladder cancer, peritoneum cancer and glioblastoma.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

### BRIEF DESCRIPTION OF THE DRAWINGS

FIG. 1: A schematic drawing of a shielded biologic by TavoPRECISE-Shield with an IGF2-based masking domain (not drawn to scale and not necessarily reflective of the relative relationship among the various domains), and the process of cleaving apart the IGF2-based masking domain by protease digestion and converting the shielded biologic by TavoPRECISE-Shield into an active biologic.

FIG. 2: A schematic diagram of a shielded biologic by TavoPRECISE-Shield with an IGF2-based masking domain, comprising a heavy chain polypeptide, comprising, from N-terminus to C-terminus, an IGF2 chain, a protease cleavable linker, and variable and constant domains of an antibody heavy chain, and a light chain polypeptide, comprising, from N-terminus to C-terminus, an IGF2 chain, a protease cleavable linker, and variable and constant domains of an antibody light chain.

FIG. 3: Reduced SDS-PAGE analysis of the heavy chain and light chain of exemplary anti-TNFα shielded antibody with IGF2-based masking domain before and after cleavage by protease MMP2.

FIG. 4: Reduced SDS-PAGE analysis of the heavy chain and light chain of exemplary anti-TNFα shielded antibody with IGF2-based masking domain before and after cleavage by protease MMP3 or uPA.

FIG. 5: Binding to human TNFα by exemplary anti-TNFα shielded antibody with IGF2-based masking domain before and after cleavage by protease MMP2, MMP3 or uPA detected by ELISA assay. The Y axes units are in Absorbance values at 450 nm. The x axes units are the concentration of the respective test articles in ng/mL units.

FIG. 6: Functional neutralization of human TNFα via HEK-Blue TNFα reporter assay by exemplary anti-TNFα shielded antibody with IGF2-based masking domain before and after cleavage by protease MMP2, MMP3 or uPA. The Y axes units are in percent TNFα activity. The x axes units are the concentration of the respective test articles in ng/mL units.

FIG. 7A. Diagram of anti-TNFα shielded antibody TAVO190197 in which the protease-cleavable linker sequence between the IGF2 A chain and antibody heavy chain comprises substrate sequence cleavable by MMP2 while the protease-cleavable linker sequence between the IGF2 B chain and antibody light chain comprises substrate sequence cleavable by MMP3. FIG. 7B. SDS-PAGE analysis of the heavy chain and light chain of TAVO190197 before and after cleavage by protease MMP2 alone, MMP3 alone or combination of MMP2 and MMP3. FIG. 7C. Binding to human TNFα by TAVO190197 before and after cleavage by protease MMP2 alone, MMP3 alone, and combination of MMP2 and MMP3. The Y axes units are in Absorbance values at 450 nm. The x axes units are the concentration of the respective test articles in ng/mL units. FIG. 7D. Functional neutralization of human TNFα by TAVO190197 before and after cleavage by protease MMP2 alone, MMP3 alone, and combination of MMP2 and MMP3. The Y axes units are in percent TNFα activity. The x axes units are the concentration of the respective test articles in ng/mL units.

FIG. 8A. ELISA assay showing binding to IGF2 receptor by anti-TNFα shielded antibody with variants IGF2-based masking domain. The Y axes units are in Absorbance values at 450 nm; the x axes units are the concentration of the respective test articles in ng/mL units. FIG. 8B. Reduced SDS-PAGE analysis of the heavy chains and light chains of anti-TNFα shielded antibody with variants IGF2-based masking domain before and after cleavage by protease MMP2. FIG. 8C. Binding to human TNFα by anti-TNFα shielded antibody with variants IGF2-based masking domain before and after cleavage by protease MMP2. The Y axes units are in Absorbance values at 450 nm; the x axes units are the concentration of the respective test articles in ng/mL units. FIG. 8D. Functional neutralization of human TNFα by anti-TNFα shielded antibody with variants IGF2-based masking domain before and after cleavage by protease MMP2. The Y axes are in percent TNFα activity. The x axes are the concentration of the respective test articles in ng/mL units.

FIG. 9A and FIG. 9C show Human TNFα binding to anti-TNFα shielded antibody with variant IGF2-based masking domain listed in Table 4. The Y axes are in units of Absorbance values at 450 nm; and the x axes are the concentration of the respective test articles in ng/mL units. FIG. 9B and FIG. 9D show functional neutralization of human TNFα by anti-TNFα shielded antibody with variant IGF2-based masking domain as listed in Table 4. The Y axes units are in percent TNFα activity; the x axes units are the concentration of the respective test articles in ng/mL units.

FIG. 10: CAIA-induced limb inflammation model showing arthritic scores upon dosing with either anti-TNFα antibodies adalimumab and infliximab, or anti-TNFα shielded antibody with IGF2-based masking domain. The Y axis is mean group arthritic score. The x axis is days post arthritis induction.

FIG. 11: Mice survival plots from the Listeria infection model upon dosing with anti-TNFα antibodies adalimumab, infliximab, and anti-TNFα shielded antibody with IGF2-based masking domain.

FIG. 12: Reduced SDS-PAGE analysis of the heavy chain and light chain of exemplary shielded anti-HER2 antibody with IGF2-based masking domain before and after cleavage by protease MMP2 or uPA.

FIG. 13: Binding to human HER2 by exemplary shielded anti-HER2 antibody with IGF2-based masking domain before and after cleavage by protease MMP2 or uPA detected by ELISA assay. The Y axes units are in Absorbance values at 450 nm. The x axes units are the concentration of the respective test articles in ng/mL.

FIG. 14: Binding to human HER2 expressed on the surface of breast cancer cell line BT474 by exemplary shielded anti-HER2 antibody with IGF2-based masking domain by flow cytometry assay. The Y axes units are in MFI. The x axes units are the concentration of the respective test articles in ng/mL units.

FIG. 15: Inhibition of the proliferation of breast cancer cell line SK-BR-3 by exemplary shielded anti-HER2 antibody with IGF2-based masking domain and matching anti-HER2 antibodies without the masking domain. The Y axes units are in percent cell viability. The x axes units are the concentration of the respective test articles in ng/mL units.

FIG. 16A. ADCC activity of exemplary shielded anti-HER2 antibody with IGF2-based masking domain and matching anti-HER2 antibody without the masking domain.

FIG. 16B. ADCP activity of exemplary shielded anti-HER2 antibody with IGF2-based masking domain and matching anti-HER2 antibody without the masking domain. In FIG. 16A and FIG. 16B, the Y axes units are in Luminescence. The x axes units are the concentration of the respective test articles in ng/mL units.

FIG. 17A. Size exclusion chromatography (SEC) analysis of exemplary anti-TNFα shielded antibody TAVO271272 with variant IGF2-based masking domain and matching anti-TNFα antibody TAVO167127 without the masking domain. FIG. 17B. Size exclusion chromatography (SEC) analysis of exemplary shielded anti-HER2 antibody TAVO293294 with variant IGF2-based masking domain and matching anti-HER2 antibody TAVO289203 without the masking domain. In both A and B, the Y axes units are in Absorbance values at 280 nm. The x axes units are retention time in minutes.

### DETAILED DESCRIPTION OF THE DISCLOSURE

### Definitions

All publications, including but not limited to patents and patent applications, cited in this specification are herein incorporated by reference as though fully set forth. If certain content of a reference cited herein contradicts or is inconsistent with the present disclosure, the present disclosure controls.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology are used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” is meant in a broad sense and includes immunoglobulin molecules including monoclonal antibodies such as murine, human, humanized and chimeric monoclonal antibodies, antibody fragments, bispecific or multi-specific antibodies, dimeric, tetrameric or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g., IgM). Each heavy chain is comprised of a heavy chain variable region (V_{H}) and a heavy chain constant region (comprised of domains C_{H1}, hinge, C_{H2} and C_{H3}). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3), are based on sequence variability (Wu et al. (1970) J Exp Med 132: 211-50 (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3), refer to the regions of antibody variable domains which are hypervariable in structure as defined by Chothia and Lesk (Chothia et al. (1987) J Mol Biol 196: 901-17. The International ImMunoGeneTics (IMGT) database (http://www_imgt_org) provides a standardized numbering and definition of antigen-binding sites. The correspondence between CDRs, HVs and IMGT delineations is described in (Lefranc et al. (2003) Dev Comp Immunol 27: 55-77). The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include F_{ab}, F(_{ab′})², F_{d} and Fᵥ fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Publ. Nos. WO1998/44001, WO1988/01649, WO1994/13804 and WO1992/01047.

“Monoclonal antibody” refers to an antibody population with single amino acid composition in each heavy and each light chain, except for possible alterations such as removal of C-terminal lysine from the antibody heavy chain. Monoclonal antibodies typically bind one antigenic epitope, except for bispecific monoclonal antibodies which bind two distinct antigenic epitopes. Monoclonal antibodies may have heterogeneous glycosylation within the antibody population. Monoclonal antibody may be monospecific or multi-specific, or monovalent, bivalent, or multivalent. A bispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a high purity, such as antibodies that are at least 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin, such as is optimized to have minimal adverse immune response when administered to a human subject. If the antibody contains a constant region or a portion of the constant region, the constant region also is derived from sequences of human origin.

“Shielded biologic by TavoPRECISE-Shield” refers to an antibody with a protein domain that masks the ability of the antibody in binding to its antigen. When the masking domain is intact, the shielded biologic by TavoPRECISE-Shield exists in an inactive state without being able to bind and exert its action to its target. The shielded biologics by TavoPRECISE-Shield can be converted to an active antibody when the masking domain is removed, and its antigen-binding capability is restored. A shielded biologic by TavoPRECISE-Shield is also referred to herein as a shielded antibody, a shielded biologic, or an antibody prodrug.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of a mammalian IgG heavy chain are designated in sequence as C_{H1}-hinge-C_{H2}-C_{H3}. The “hinge,” “hinge region” or “hinge domain” of an IgG is generally defined as including Glu216 and terminating at Pro230 of human IgG₁ according to the EU Index but functionally, the flexible portion of the chain may be considered to include additional residues termed the upper and lower hinge regions, such as from Glu216 to Gly237 and the lower hinge has been referred to as residues 233 to 239 of the Fc region where F_{c}γR binding was generally attributed. Hinge regions of other IgG isotypes may be aligned with the IgG₁ sequence by placing the first and last cysteine residues forming inter-heavy chain S—S bonds. Although boundaries may vary slightly, as numbered according to the EU Index, the CH₁ domain is adjacent to the V_{H} domain and amino terminal to the hinge region of an immunoglobulin heavy chain molecule and includes the first (most amino terminal) constant region of an immunoglobulin heavy chain, e.g., from about EU positions 118-215. The F_{c} domain extends from amino acid 231 to amino acid 447; the C_{H2} domain is from about Ala231 to Lys340 or Gly341 and the C_{H3} from about Gly341 or Gln342 to Lys447. The residues of the IgG heavy chain constant region of the C_{H1} region terminate at Lys. An Fc domain containing molecule comprises at least the C_{H2} and the C_{H3} domains of an antibody constant region, and therefore comprises at least a region from about Ala231 to Lys447 of IgG heavy chain constant region. The Fc domain containing molecule may optionally comprise at least a portion of the hinge region.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in a 3-dimensional space through the folding of the antigen molecule. Antibody “epitope” depends on the methodology used to identify the epitope.

A “leader sequence” as used herein includes any signal peptide that can be processed by a mammalian cell, including the human B2M leader. Such sequences are well-known in the art.

A “cleavable linker” is a peptide substrate cleavable, e.g., by an enzyme. For example, a cleavable linker, upon being cleaved by an enzyme, allows for activation of the present shielded biologics by TavoPRECISE-Shield via removal of the IGF2-based masking domain. Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (e.g., conservative in nature as would be known to a person in the art) to the native sequence, as long as the protein maintains a desired activity. These modifications can be deliberate, as through site-directed mutagenesis, or can be accidental, such as through mutations of hosts that produce the proteins, or errors due to PCR amplification or other recombinant DNA methods.

The term “masking domain” in this disclosure refers to a protein domain that can be fused to an antibody and mask the antibody from binding to its antigen. The masking domain can shield the antibody in recognizing its target so that the antibody is kept in an inactive shielded biologic by TavoPRECISE-Shield form. When the masking domain is removed, the variable domains of the antibody are exposed and can bind and exert actions to its target.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

“Valent” refers to the presence of a specified number of binding sites specific for an antigen in a molecule. As such, the terms “monovalent,” “bivalent,” “tetravalent,” and “hexavalent” refer to the presence of one, two, four and six binding sites, respectively, specific for an antigen in a molecule.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence, in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced, and the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refer to an antibody binding to a specific antigen with greater affinity than for another antigen. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and/or (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to an amount of an agent, or combined amounts of two agents, that, when administered to a mammal or other subject for treating a disease, is sufficient to affect such treatment for the disease. The “therapeutically effective amount” will vary depending on the agent(s), the disease and its severity, and the age, weight, etc., of the subject to be treated.

Before the present disclosure is further described, it is to be understood that this disclosure is not limited to particular embodiments described, as such may, of course, vary. It is also to be understood that the terminology used herein is for the purpose of describing particular embodiments only and is not intended to be limiting.

Shielded Biologics by TavoPRECISE-Shield with Masking Domains

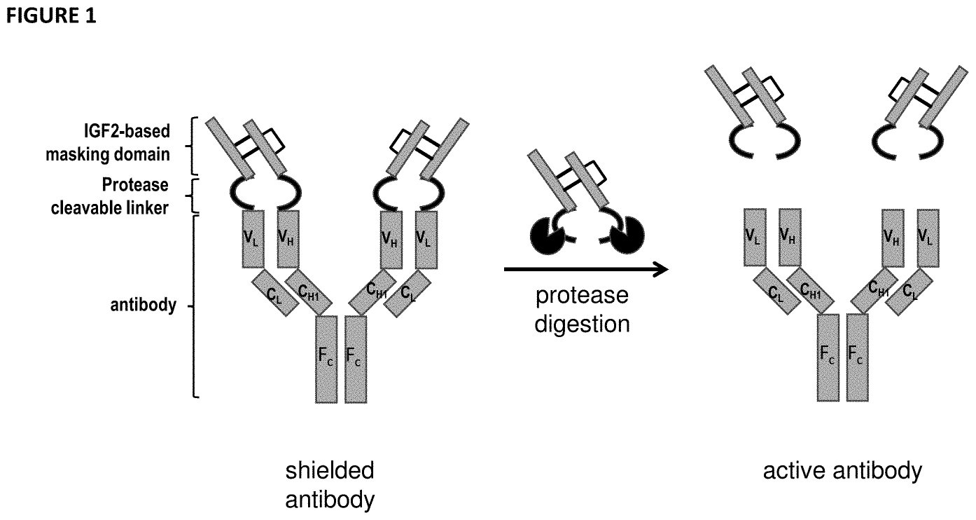

Therapeutic antibodies hold great promise as an efficacious treatment for cancer, immunological diseases, infectious diseases, and metabolic diseases. However, mechanism-based on-target, off-site toxicities associated with systematic administration of antibody drugs to a target, which has important physiological roles in normal tissues besides pathological roles in disease states, greatly limit the choice of these therapeutic antibodies as treatment options or limit high therapeutic doses. This disclosure provides a novel antibody engineering approach to generate a shielded biologic by TavoPRECISE-Shield in which a masking domain is fused to a therapeutic biologic via a protease-cleavable linker. The masking domain is employed to mask the antigen-binding capability of the biologics by steric hindrance in normal tissues. In disease tissues, overexpressed proteases can cleave off the masking domain, so the shielded biologics by TavoPRECISE-Shield is converted to an active biologic to bind and exert its functional activity to its target (see, e.g., FIG. 1). This shielded biologics by TavoPRECISE-Shield approach can facilitate an enhanced site-of-action selectivity of the biologics by acting only on targets in disease tissues while sparing targets in normal tissues, thus helping to improve the safety profile of the biologics by avoiding systemic toxicity.

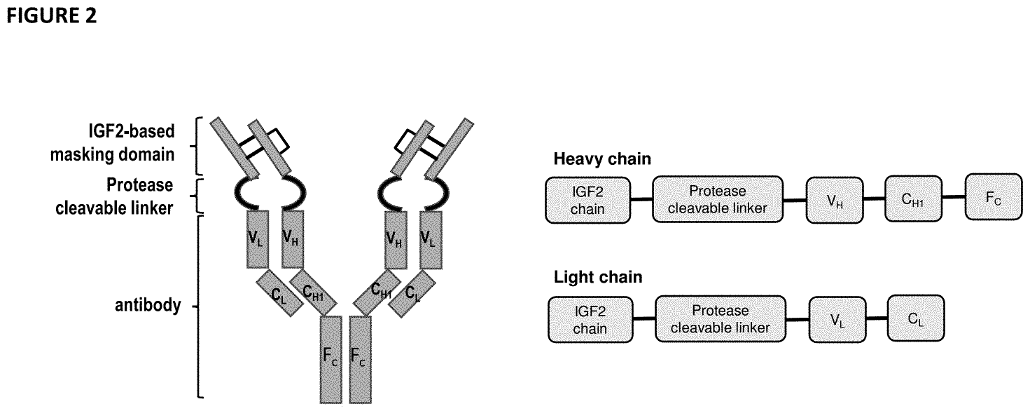

In some embodiments, the disclosure provides a masking domain comprising two peptide chains (e.g., a first masking domain unit and a second masking domain unit), referred to as the A chain and B chain, of mature Insulin Growth Factor 2 (IGF2). The IGF2 A chain and B chain are linked together by one or more, e.g., two, inter-molecular disulfide bonds, and an additional intra-molecular disulfide bond is formed within the A chain (see, e.g., FIG. 1). A shielded biologic by TavoPRECISE-Shield is formed with the A chain and B chain of IGF2 fused to the N-terminus of the antibody heavy chain and light chain respectively via protease-cleavable linker sequences. The protease-cleavable linker sequence comprises one or more substrate sequence of proteases, optionally also comprising one or more linker peptides (also known as spacers) between IGF2 chains and protease substrate sequences and/or between protease substrate sequences and antibody chains. As shown in FIG. 2, a shielded biologic by TavoPRECISE-Shield comprises two IGF2-based masking domains (formed by two sets of a first and a second masking domain units) fused to the N-terminus of the two Fab domains of the antibody via four protease-cleavable linker sequences.

In certain embodiments, a shielded biologic by TavoPRECISE-Shield comprises: a) two heavy chain polypeptides comprising, in an N-terminus to C-terminus orientation, the A chain of IGF2, protease-cleavable linker and antibody heavy chain; and b) two light chain polypeptides comprising, in an N-terminus to C-terminus orientation, the B chain of IGF2, protease-cleavable linker and antibody light chains (see, e.g., FIG. 2).

In certain embodiments, a shielded biologic by TavoPRECISE-Shield comprises: a) two heavy chain polypeptides comprising, in an N-terminus to C-terminus orientation, the B chain of IGF2, protease-cleavable linker and antibody heavy chains; and b) two light chain polypeptides comprising, in an N-terminus to C-terminus orientation, the A chain of IGF2, protease-cleavable linker and antibody light chains (see, e.g., FIG. 2).

As a non-limiting example, the disclosure provides a shielded biologic by TavoPRECISE-Shield TAVO190191 in which IGF2-based masking domains are fused, via MMP2/9 protease-cleavable linker sequences, to the N-terminus of an anti-TNFα antibody. TAVO190191 comprises a heavy chain polypeptide, designated as EAC190 with sequence set forth as SEQ ID NO. 1, comprising, in an N-terminus to C-terminus orientation, the A chain of IGF2, MMP2/9 protease-cleavable linker sequence and an anti-TNFα antibody heavy chain sequence with IgG1 Fc with E233P, L234A, L235A, F405L, M428L, N434S mutations and G236 deleted, and a light chain polypeptide, designated as EAC191 with sequence set forth as SEQ ID NO. 2, comprising, in an N-terminus to C-terminus orientation, the B chain of IGF2, MMP2/9 protease-cleavable linker sequence and an anti-TNFα antibody light chain sequence (Table 2).

As a non-limiting example, the disclosure provides a shielded biologic by TavoPRECISE-Shield TAVO196197 in which IGF2-based masking domains are fused, via MMP3 protease-cleavable linker sequences, to the N-terminus of an anti-TNFαantibody. TAVO196197 comprises a heavy chain polypeptide, designated as EAC196 with sequence set forth as SEQ ID NO. 3, comprising, in an N-terminus to C-terminus orientation, the A chain of IGF2, MMP3 protease-cleavable linker sequence and an anti-TNFα antibody heavy chain sequence with IgG1 Fc with E233P, L234A, L235A, F405L, M428L, N434S mutations and G236 deleted, and a light chain polypeptide, designated as EAC197 with sequence set forth as SEQ ID NO. 4, comprising, in an N-terminus to C-terminus orientation, the B chain of IGF2, MMP3 protease-cleavable linker sequence and an anti-TNFα antibody light chain sequence (Table 2).

As a non-limiting example, the disclosure provides a shielded biologic by TavoPRECISE-Shield TAVO271272 in which variant IGF2-based masking domains are fused, via MMP2/9 protease-cleavable linker sequences, to the N-terminus of an anti-TNFα antibody. TAVO271272 comprises a heavy chain polypeptide, designated as EAC271 with sequence set forth as SEQ ID NO. 73, comprising, in an N-terminus to C-terminus orientation, the A chain of IGF2 with V43L mutation, MMP2/9 protease-cleavable linker sequence and an anti-TNFα antibody heavy chain sequence with IgG1 Fc with E233P, L234A, L235A, F405L, M428L, N434S mutations and G236 deleted, and a light chain polypeptide, designated as EAC272 with sequence set forth as SEQ ID NO. 74, comprising, in an N-terminus to C-terminus orientation, the B chain of IGF2 with Y27A mutation, MMP2/9 protease-cleavable linker sequence and an anti-TNFα antibody light chain sequence (Table 4).

As a non-limiting example, the disclosure provides a shielded biologic by TavoPRECISE-Shield TAVO212213 in which IGF2-based masking domains are fused, via MMP2/9 protease-cleavable linker sequences, to the N-terminus of an anti-IL-1β antibody. TAVO212213 comprises a heavy chain polypeptide, designated as EAC212 with sequence set forth as SEQ ID NO. 5, comprising, in an N-terminus to C-terminus orientation, the A chain of IGF2, MMP2/9 protease-cleavable linker sequence and an anti-IL-1β antibody heavy chain sequence with IgG1 Fc with E233P, L234A, L235A, K409R, M428L, N434S mutations and G236 deleted, and a light chain polypeptide, designated as EAC213 with sequence set forth as SEQ ID NO. 6, comprising, in an N-terminus to C-terminus orientation, the B chain of IGF2, MMP2/9 protease-cleavable linker sequence and an anti-IL-1β antibody light chain sequence (Table 2).

As a non-limiting example, the disclosure provides a shielded biologic by TavoPRECISE-Shield TAVO214215 in which IGF2-based masking domains are fused, via MMP3 protease-cleavable linker sequences, to the N-terminus of an anti-IL-1 antibody. TAVO214215 comprises a heavy chain polypeptide, designated as EAC214 with sequence set forth as SEQ ID NO. 7, comprising, in an N-terminus to C-terminus orientation, the A chain of IGF2, MMP3 protease-cleavable linker sequence and an anti-IL-1β antibody heavy chain sequence with IgG1 Fc with E233P, L234A, L235A, K409R, M428L, N434S mutations and G236 deleted, and a light chain polypeptide, designated as EAC215 with sequence set forth as SEQ ID NO. 8, comprising, in an N-terminus to C-terminus orientation, the B chain of IGF2, MMP3 protease-cleavable linker sequence and an anti-IL-1β antibody light chain sequence (Table 2).

As a non-limiting example, the disclosure provides a shielded biologic by TavoPRECISE-Shield TAVO224225 in which IGF2-based masking domains are fused, via MMP2/9 protease-cleavable linker sequences, to the N-terminus of anti-HER2 antibody Trastuzumab. TAVO224225 comprises a heavy chain polypeptide, designated as EAC224 with sequence set forth as SEQ ID NO. 9, comprising, in an N-terminus to C-terminus orientation, the A chain of IGF2, MMP2/9 protease-cleavable linker sequence and Trastuzumab heavy chain sequence with IgG1 Fc with E233P, F405L, M428L, N434S mutations and G236 deleted, and a light chain polypeptide, designated as EAC225 with sequence set forth as SEQ ID NO. 10, comprising, in an N-terminus to C-terminus orientation, the B chain of IGF2, MMP2/9 protease-cleavable linker sequence and Trastuzumab light chain sequence (Table 2).

As a non-limiting example, the disclosure provides a shielded biologic by TavoPRECISE-Shield TAVO243244 in which IGF2-based masking domains are fused, via uPA protease-cleavable linker sequences, to the N-terminus of anti-HER2 antibody Trastuzumab. TAVO243244 comprises a heavy chain polypeptide, designated as EAC243 with sequence set forth as SEQ ID NO. 11, comprising, in an N-terminus to C-terminus orientation, the A chain of IGF2, uPA protease-cleavable linker sequence and Trastuzumab heavy chain sequence with IgG1 Fc with E233P, F405L, M428L, N434S mutations and G236 deleted, and a light chain polypeptide, designated as EAC244 with sequence set forth as SEQ ID NO. 12, comprising, in an N-terminus to C-terminus orientation, the B chain of IGF2, uPA protease-cleavable linker sequence and Trastuzumab light chain sequence (Table 2).

As a non-limiting example, the disclosure provides a shielded biologic by TavoPRECISE-Shield TAVO293294 in which variant IGF2-based masking domains are fused, via MMP2/9 protease-cleavable linker sequences, to the N-terminus of anti-HER2 antibody Trastuzumab. TAVO293294 comprises a heavy chain polypeptide, designated as EAC293 with sequence set forth as SEQ ID NO. 80, comprising, in an N-terminus to C-terminus orientation, the A chain of IGF2 with V43L mutation, MMP2/9 protease-cleavable linker sequence and Trastuzumab heavy chain sequence with IgG1 Fc with F405L, M428L, N434S mutations, and a light chain polypeptide, designated as EAC294 with sequence set forth as SEQ ID NO. 81, comprising, in an N-terminus to C-terminus orientation, the B chain of IGF2 with Y27A mutation, MMP2/9 protease-cleavable linker sequence and Trastuzumab light chain sequence (Table 5).

As a non-limiting example, the disclosure provides a shielded biologic by TavoPRECISE-Shield TAVO259229 in which IGF2-based masking domains are fused, via MMP2/9 protease-cleavable linker sequences, to the N-terminus of anti-VEGF antibody Bevacizumab. TAVO259229 comprises a heavy chain polypeptide, designated as EAC259 with sequence set forth as SEQ ID NO. 13, comprising, in an N-terminus to C-terminus orientation, the A chain of IGF2, MMP2/9 protease-cleavable linker sequence and Bevacizumab heavy chain sequence with IgG1 Fc with E233P, K409R, M428L, N434S mutations and G236 deleted, and a light chain polypeptide, designated as EAC229 with sequence set forth as SEQ ID NO. 14, comprising, in an N-terminus to C-terminus orientation, the B chain of IGF2, MMP2/9 protease-cleavable linker sequence and Bevacizumab light chain sequence (Table 2).

As a non-limiting example, the disclosure provides a shielded biologic by TavoPRECISE-Shield TAVO245246 in which IGF2-based masking domains are fused, via uPA protease-cleavable linker sequences, to the N-terminus of anti-VEGF antibody Bevacizumab. TAVO245246 comprises a heavy chain polypeptide, designated as EAC245 with sequence set forth as SEQ ID NO. 15, comprising, in an N-terminus to C-terminus orientation, the A chain of IGF2, uPA protease-cleavable linker sequence and Bevacizumab heavy chain sequence with IgG1 Fc with E233P, F405L, M428L, N434S mutations and G236 deleted, and a light chain polypeptide, designated as EAC246 with sequence set forth as SEQ ID NO. 16, comprising, in an N-terminus to C-terminus orientation, the B chain of IGF2, uPA protease-cleavable linker sequence and Bevacizumab light chain sequence (Table 2).

TABLE 2
Example shielded biologics by TavoPRECISE-Shield with IGF2-based masking domain

| | | | | Protease | |
|---|---|---|---|---|---|
| Shielded biologies by | Heavy | Light | | substrate | |
| TavoPRECISE-Shield | chain | chain | target | site | Fc |
| TAVO190191 | EAC190 | EAC191 | TNFα | MMP2/9 | IgG1 with E233P, |
| | | | | | L234A, L235A, F405L, |
| | | | | | M428L, N434S |
| | | | | | mutations and G236 |
| | | | | | deleted |
| TAVO196197 | EAC196 | EAC197 | TNFα | MMP3 | IgG1 with E233P, |
| | | | | | L234A, L235A, F405L, |
| | | | | | M428L, N434S |
| | | | | | mutations and G236 |
| | | | | | deleted |
| TAVO212213 | EAC212 | EAC213 | IL-β | MMP2/9 | IgG1 with E233P, |
| | | | | | L234A, L235A, K409R, |
| | | | | | M428L, N434S |
| | | | | | mutations and G236 |
| | | | | | deleted |
| TAVO214215 | EAC214 | EAC215 | IL-β | MMP3 | IgG1 with E233P, |
| | | | | | L234A, L235A, K409R, |
| | | | | | M428L, N434S |
| | | | | | mutations and G236 |
| | | | | | deleted |
| TAVO224225 | EAC224 | EAC225 | HER2 | MMP2/9 | IgG1 with E233P, |
| | | | | | F405L, M428L, N434S |
| | | | | | mutations and G236 |
| | | | | | deleted |
| TAVO243244 | EAC243 | EAC244 | HER2 | uPA | IgG1 with E233P, |
| | | | | | F405L, M428L, N434S |
| | | | | | mutations and G236 |
| | | | | | deleted |
| TAVO259229 | EAC259 | EAC229 | VEGF | MMP2/9 | IgG1 with E233P, |
| | | | | | K409R, M428L, N434S |
| | | | | | mutations and G236 |
| | | | | | deleted |
| TAVO245246 | EAC245 | EAC246 | VEGF | uPA | IgG1 with E233P, |
| | | | | | F405L, M428L, N434S |
| | | | | | mutations and G236 |
| | | | | | deleted |
| TAVO190197 | EAC190 | EAC197 | TNFα | MMP2/9 | IgG1 with E233P, |
| | | | | MMP3 | L234A, L235A, F405L, |
| | | | | | M428L, N434S |
| | | | | | mutations and G236 |
| | | | | | deleted |
| TAVO224244 | EAC224 | EAC244 | HER2 | MMP2/9 | IgG1 with E233P, |
| | | | | uPA | F405L, M428L, N434S |
| | | | | | mutations and G236 |
| | | | | | deleted |
| TAVO259229x245246 | EAC259 | EAC229 | VEGF | MMP2/9 | IgG1 with E233P, |
| | EAC245 | EAC246 | | uPA | F405L, K409R, M428L, |
| | | | | | N434S mutations and |
| | | | | | G236 deleted |
| TAVO243244x259229 | EAC243 | EAC244 | HER2 | MMP2/9 | IgG1 with E233P, |
| | EAC259 | EAC229 | VEGF | uPA | F405L, K409R, M428L, |
| | | | | | N434S mutations and |
| | | | | | G236 deleted |
| TAVO190191x212213 | EAC190 | EAC191 | TNFα | MMP2/9 | IgG1 with E233P, |
| | EAC212 | EAC213 | IL-1β | | L234A, L235A, F405L, |
| | | | | | K409R, M428L, N434S |
| | | | | | mutations and G236 |
| | | | | | deleted |
| TAVO224225x259229 | EAC224 | EAC225 | HER2 | MMP2/9 | IgG1 with E233P, |
| | EAC259 | EAC229 | VEGF | | F405L, K409R, M428L, |
| | | | | | N434S mutations and |
| | | | | | G236 deleted |

### Masking Domains (e.g., IGF2-Based)

Insulin Growth Factor 2 (IGF2) is a protein hormone that shares structure similarity to Insulin. The mature, active form of IGF2 is a heterodimeric peptide comprised with a shorter A chain and a longer B chain. The two peptide chains dimerize to each other by two inter-molecular disulfide bonds formed between two cysteines on each peptide chain. The A chain peptide contains another pair of cysteines which forms an intra-molecular disulfide bond within A chain. The active heterodimeric IGF2 is formed by posttranslational modifications from a much longer single chain pre-prohormone comprising, in an N-terminus to C-terminus orientation, a signal peptide, B chain, C chain and A chain. The signal peptide in pre-prohormones leads the transport of the peptide from the ribosomal ubiquitins. Following the loss of signal peptide, the pre-prohormone is converted to a prohormone via co-translational modifications. Proteolytic cleavage of the C-chain then yields the A- and B-chain combination, forming mature, active 2-chain heterodimeric peptide.

In certain embodiments, IGF2 pre-prohormone comprises an amino acid sequence having at least 70%, at least 75%, at least 80%, at least 85%, at least 90%, at least 95%, at least 98%, at least 99%, or 100%, amino acid sequence identity to human IGF2 pre-prohormone sequence as SEQ ID NO: 17.

In certain embodiments, the A chain of IGF2 comprises an amino acid sequence having at least 70%, at least 75%, at least 80%, at least 85%, at least 90%, at least 95%, at least 98%, at least 99%, or 100%, amino acid sequence identity to the A chain of human IGF2 sequence as SEQ ID NO: 18.

In certain embodiments, the B chain of IGF2 comprises an amino acid sequence having at least 70%, at least 75%, at least 80%, at least 85%, at least 90%, at least 95%, at least 98%, at least 99%, or 100%, amino acid sequence identity to the B chain of human IGF2 sequence as SEQ ID NO: 19.

In certain embodiments, the C chain of IGF2 comprises an amino acid sequence having at least 70%, at least 75%, at least 80%, at least 85%, at least 90%, at least 95%, at least 98%, at least 99%, or 100%, amino acid sequence identity to the C chain of human IGF2 sequence as SEQ ID NO: 20.

The disclosure provides a masking domain comprising the heterodimerized IGF2 peptides. In some embodiments, the two IGF2 peptide chains dimerize to each other by one or more, e.g., two, inter-molecular disulfide bonds formed between two cysteines on A chain and B chain. In a shielded biologic by TavoPRECISE-Shield, the A chain and B chain of IGF2 are fused to the N-terminus of antibody heavy chain and light chain respectively via a protease-cleavable linker sequence. Due to the size of heterodimerized IGF2 peptides and close proximity to the Fab domains of the antibody, the IGF2-based masking domain interferes or blocks the binding of the antibody to its antigen. In certain embodiments, the A chain of IGF2 is fused to the N-terminus of antibody heavy chain and B chain of IGF2 is fused to the N-terminus of antibody light chain. In other embodiments, the B chain of IGF2 is fused to the N-terminus of antibody heavy chain and A chain of IGF2 is fused to the N-terminus of antibody light chain.

The disclosure provides an IGF2-based masking domain comprising with the A chain of IGF2 comprising an amino acid sequence having at least 70%, at least 75%, at least 80%, at least 85%, at least 90%, at least 95%, at least 98%, at least 99%, or 100%, amino acid sequence identity to the A chain of human IGF2 sequence as SEQ ID NO: 18, and with the B chain of IGF2 comprising an amino acid sequence having at least 70%, at least 75%, at least 80%, at least 85%, at least 90%, at least 95%, at least 98%, at least 99%, or 100%, amino acid sequence identity to the B chain of human IGF2 sequence as SEQ ID NO: 19.

In certain embodiments, the IGF2-based masking domain comprises a truncated form of the A chain of human IGF2 sequence as SEQ ID NO: 18. In other embodiments, the IGF2-based masking domain comprises the A chain of human IGF2 sequence as SEQ ID NO: 18 and additional sequences flanking the A chain from human IGF2 pre-prohormone sequence as SEQ ID NO: 17.

In certain embodiments, the IGF2-based masking domain comprises a truncated form of the B chain of human IGF2 sequence as SEQ ID NO: 19. In other embodiments, the IGF2-based masking domain comprises the B chain of human IGF2 sequence as SEQ ID NO: 19 and additional sequences flanking the B chain from human IGF2 pre-prohormone sequence as SEQ ID NO: 17.

In certain embodiments, the IGF2-based masking domain comprises the A chain and B chain of human IGF2 with mutations that disrupt IGF2 binding to its receptors, thus reduce any toxic effects, including aberrant modulation of glucose metabolism and cell proliferation, due to IGF2 signaling. The A chain and B chain of human IGF2 with mutations that disrupt IGF2 binding to its receptors includes but not limited to mutated A chain and B chain sequences as SEQ ID NO: 69, 70, 71, 72.

In certain embodiments, the IGF2-based masking domain comprises the human IGF2 A chain with V43L mutation and human IGF2 B chain with Y27A mutation that disrupt IGF2 binding to its receptors, thus reduce any toxic effects, including aberrant modulation of glucose metabolism and cell proliferation, due to IGF2 signaling.

In certain embodiments, the IGF2-based masking domain comprises the A chain of IGF2 of other non-human mammalian species, e.g., non-human primates, canines, felines, lagomorphs, ungulates (e.g., equines, bovines, ovines, caprines, etc.), rodents, and the like.

In certain embodiments, the IGF2-based masking domain comprises the B chain of IGF2 of other non-human mammalian species, e.g., non-human primates, canines, felines, lagomorphs, ungulates (e.g., equines, bovines, ovines, caprines, etc.), rodents, and the like.

IGF2 is a mitogenic growth factor (Klement and Fink 2016). It binds to three receptor complexes namely the insulin receptor isoform A (IR-A), IGF1 receptor (IGF1R), and IGF2 receptor (IGF2R). IGF2 exerts its effect on cell growth, differentiation, and survival through binding to IR-A and IGF1R receptors. However, the growth-promoting function of IGF2 is limited to embryonic development in mammals, in contrast to Insulin-like growth factor 1 (IGF1) which is a major growth factor in adults. By acting as a major fetal growth hormone in mammals, IGF2 plays a key role in regulating fetoplacental development. IGF2 promotes granulosa cell proliferation during the follicular phase of the menstrual cycle, acting alongside follicle stimulating hormone. After ovulation has occurred, IGF-2 promotes progesterone secretion during the luteal phase of the menstrual cycle, together with luteinizing hormone. In adults, IGF2 is involved in glucose metabolism in adipose tissue, skeletal muscle, and liver, but its role is not as important as insulin or IGF1. Besides IR-A and IGF1R receptors, IGF2 can bind to IGF2R, but this binding is non-signaling and leads to the sequestration of IGF2 for internalization and degradation, so IGF2R acts as a scavenger.

Mature IGF2 is produced mainly by the liver, and it is also secreted by most tissues where it can act in an autocrine or paracrine manner. The concentration of circulating IGF2 in blood is about 400-700 ng/mL which is the dominant form of IGF in blood and over three times more abundant than that of IGF1 (Yu, Mistry et al. 1999). Circulating IGF2 exists in complexes bound to IGF binding proteins. Currently, at least six high affinity insulin-like growth-factor-binding proteins (IGFBPs) have been identified, which regulate the circulating concentration of IGF2. Over-expression of IGF2 occurs in many cancers associated with a poor prognosis (Livingstone 2013). However, the causal relationship between IGF2 and tumor progression remain elusive. In addition, an experimental bispecific antibody Dusigitumab (MEDI-573) targeting to inhibit both IGF1 and IGF2 showed minimal anti-tumor efficacy in a Phase I/II clinical trials (Haluska, Menefee et al. 2014).

IGF2 belongs to Insulin-Relaxin superfamily of peptide hormones (Nair, Samuel et al. 2012). This superfamily includes Insulin family of peptides, comprising Insulin, IGF1 and IGF2. Within this family is also the relaxin subfamily which comprises seven members: relaxin-1, -2 and -3 and insulin-like peptides 3 (INSL3), 4 (INSL4), 5 (INSL5) and 6 (INSL6). Different members of this superfamily share structural similarities although their sequences are quite diverse from each other. The mature peptide hormones of all members have four conserved cysteines in the A chain and two conserved cysteines in the B chain. Two cysteines from the A chain and two cysteines from the B chain form the inter-chain disulfide bonds while the two additional cysteines in the A chain from the intra-chain disulfide bond. Similar to IGF2, all these family member peptide hormones undergo a series of post-translational modification during maturation, evolving from a single chain preprohormone to active heterodimeric mature form.

Considering the structural similarity among member peptides within this Insulin-Relaxin superfamily, the A chain and B chain of other family member peptides can also constitute the masking domain instead of the IGF2-based masking domain illustrated in this disclosure. Due to the size of heterodimerized peptides and close proximity to the Fab domains of the antibody, the masking domain based on other peptide hormones within the Insulin-Relaxin superfamily may interfere or block the binding of the antibody to its antigen.

In some embodiments, the disclosure provides a shielded biologic by TavoPRECISE-Shield with the A chain and B chain of Insulin fused to the N-terminus of an antibody heavy chain and a light chain respectively via a protease-cleavable linker sequence. The heterodimerized Insulin peptides comprise the masking domain of a shielded biologic by TavoPRECISE-Shield. In some instances, the A chain and B chain of Insulin are derived from Insulin pre-prohormone comprising an amino acid sequence having at least 70%, at least 75%, at least 80%, at least 85%, at least 90%, at least 95%, at least 98%, at least 99%, or 100%, amino acid sequence identity to human Insulin pre-prohormone sequence set forth as SEQ ID NO: 21.

In some embodiments, the disclosure provides a shielded biologic by TavoPRECISE-Shield with the A chain and B chain of IGF1 fused to the N-terminus of an antibody heavy chain and a light chain respectively via a protease-cleavable linker sequence. The heterodimerized IGF1 peptides comprise the masking domain of a shielded biologic by TavoPRECISE-Shield. In some instances, the A chain and B chain of IGF1 are derived from IGF1 pre-prohormone comprising an amino acid sequence having at least 70%, at least 75%, at least 80%, at least 85%, at least 90%, at least 95%, at least 98%, at least 99%, or 100%, amino acid sequence identity to human IGF1 pre-prohormone sequence set forth as SEQ ID NO: 22.

In some embodiments, the disclosure provides a shielded biologic by TavoPRECISE-Shield with the A chain and B chain of Relaxin-1 fused to the N-terminus of an antibody heavy chain and a light chain respectively via a protease-cleavable linker sequence. The heterodimerized Relaxin-1 peptides comprise the masking domain of a shielded biologic by TavoPRECISE-Shield. In some instances, the A chain and B chain of Relaxin-1 are derived from Relaxin-1 pre-prohormone comprising an amino acid sequence having at least 70%, at least 75%, at least 80%, at least 85%, at least 90%, at least 95%, at least 98%, at least 99%, or 100%, amino acid sequence identity to human Relaxin-1 pre-prohormone sequence set forth as SEQ ID NO: 23.

In some embodiments, the disclosure provides a shielded biologic by TavoPRECISE-Shield with the A chain and B chain of Relaxin-2 fused to the N-terminus of antibody heavy chain and light chain respectively via a protease-cleavable linker sequence. The heterodimerized Relaxin-2 peptides comprise the masking domain of a shielded biologic by TavoPRECISE-Shield. In some instances, the A chain and B chain of Relaxin-2 are derived from Relaxin-2 pre-prohormone comprising an amino acid sequence having at least 70%, at least 75%, at least 80%, at least 85%, at least 90%, at least 95%, at least 98%, at least 99%, or 100%, amino acid sequence identity to human Relaxin-2 pre-prohormone sequence set forth as SEQ ID NO: 24.

In some embodiments, the disclosure provides a shielded biologic by TavoPRECISE-Shield with the A chain and B chain of Relaxin-3 fused to the N-terminus of an antibody heavy chain and a light chain respectively via a protease-cleavable linker sequence. The heterodimerized Relaxin-3 peptides comprise the masking domain of a shielded biologic by TavoPRECISE-Shield. In some instances, the A chain and B chain of Relaxin-3 are derived from Relaxin-3 pre-prohormone comprising an amino acid sequence having at least 70%, at least 75%, at least 80%, at least 85%, at least 90%, at least 95%, at least 98%, at least 99%, or 100%, amino acid sequence identity to human Relaxin-3 pre-prohormone sequence set forth as SEQ ID NO: 25.

In some embodiments, the disclosure provides a shielded biologic by TavoPRECISE-Shield with the A chain and B chain of INSL3 fused to the N-terminus of an antibody heavy chain and a light chain respectively via a protease-cleavable linker sequence. The heterodimerized INSL3 peptides comprise the masking domain of a shielded biologic by TavoPRECISE-Shield. In some instances, the A chain and B chain of INSL3 are derived from INSL3 pre-prohormone comprising an amino acid sequence having at least 70%, at least 75%, at least 80%, at least 85%, at least 90%, at least 95%, at least 98%, at least 99%, or 100%, amino acid sequence identity to human INSL3 pre-prohormone sequence set forth as SEQ ID NO: 26.

In some embodiments, the disclosure provides a shielded biologic by TavoPRECISE-Shield with the A chain and B chain of INSL4 fused to the N-terminus of antibody heavy chain and light chain respectively via a protease-cleavable linker sequence. The heterodimerized INSL4 peptides comprise the masking domain of a shielded biologic by TavoPRECISE-Shield. In some instances, the A chain and B chain of INSL4 are derived from INSL4 pre-prohormone comprising an amino acid sequence having at least 70%, at least 75%, at least 80%, at least 85%, at least 90%, at least 95%, at least 98%, at least 99%, or 100%, amino acid sequence identity to human INSL4 pre-prohormone sequence set forth as SEQ ID NO: 27.

In some embodiments, the disclosure provides a shielded biologic by TavoPRECISE-Shield with the A chain and B chain of INSL5 fused to the N-terminus of an antibody heavy chain and a light chain respectively via a protease-cleavable linker sequence. The heterodimerized INSL5 peptides comprise the masking domain of a shielded biologic by TavoPRECISE-Shield. In some instances, the A chain and B chain of INSL5 are derived from INSL5 pre-prohormone comprising an amino acid sequence having at least 70%, at least 75%, at least 80%, at least 85%, at least 90%, at least 95%, at least 98%, at least 99%, or 100%, amino acid sequence identity to human INSL5 pre-prohormone sequence set forth as SEQ ID NO: 28.

In some embodiments, the disclosure provides a shielded biologic by TavoPRECISE-Shield with the A chain and B chain of INSL6 fused to the N-terminus of an antibody heavy chain and a light chain respectively via a protease-cleavable linker sequence. The heterodimerized INSL6 peptides comprise the masking domain of a shielded biologic by TavoPRECISE-Shield. In some instances, the A chain and B chain of INSL6 are derived from INSL6 pre-prohormone comprising an amino acid sequence having at least 70%, at least 75%, at least 80%, at least 85%, at least 90%, at least 95%, at least 98%, at least 99%, or 100%, amino acid sequence identity to human INSL6 pre-prohormone sequence set forth as SEQ ID NO: 29.

Many member peptide hormones in this Insulin-Relaxin superfamily play important and diverse physiological roles. Insulin is critical for the maintaining of blood glucose level and IGF1 is a growth hormone in adults. All seven members of the Relaxin family of peptide hormones play diverse physiological roles in different tissues (Nair, Samuel et al. 2012). In comparison to other peptide hormones, IGF2 does not play a significant physiological role in the adult stage and its circulation level in serum is high, which makes IGF2 a better choice as a masking domain in a shielded biologic by TavoPRECISE-Shield.

### Protease-Cleavable Linker

The protease-cleavable linker is a peptide substrate cleavable by a protease linking a masking domain (e.g., IGF2-based) and antibody heavy and light chains. For example, it comprises one or more substrate sequences of protease, and optionally, it also comprises one or more linker peptides between IGF2 chains and protease substrate sequences and/or between protease substrate sequences and antibody chains. For each of the two Fab arm domains of the antibody, either A chain or B chain of IGF2 is fused to the N-terminus of the antibody heavy chain via one protease-cleavable linker and the counterpart B chain or A chain of IGF2 is fused to the N-terminus of the antibody light chain via another protease-cleavable linker. The A chain and B chain of IGF2 associated with one Fab arm heterodimerize to each other via two disulfide bonds to form one masking domain. As exemplified in FIG. 2, the shielded biologic by TavoPRECISE-Shield comprises two IGF2-based masking domains fused to the N-terminus of the two Fab domains of the antibody via four protease-cleavable linker sequences.

Many disease tissues, including tumor microenvironment and inflammation site, are abundant with various types of proteases whose overexpression correlate with the disease progression. In disease tissues, the protease-cleavable linker sequences of the shielded biologics by TavoPRECISE-Shield are recognized by specific proteases, and the peptide sequence linking the IGF2 chains and antibody chains is cleaved apart. For each Fab arm of the shielded biologics by TavoPRECISE-Shield, the protease may cleave apart both of the two protease-cleavable linker sequences, so the IGF2-based masking domains are “cleaved off” completely. Alternatively, the protease may cleave apart only one of the two protease-cleavable linker sequences, so the IGF2-based masking domain is “cleaved open”. In either case, the masking domain is no longer able to interfere or block the binding of the Fab arm to its target antigen. As a result of protease cleavage, the shielded biologics by TavoPRECISE-Shield can be converted into an active antibody to bind and exert its functional activity on its target (FIG. 1).

In some embodiments, the four protease-cleavable linker sequences linking the two IGF2-based masking domains and the two Fab domains in the shielded biologics by TavoPRECISE-Shield comprise the same sequence with substrate sequence cleavable by the same type of protease. As a non-limiting example, the disclosure provides a shielded biologic by TavoPRECISE-Shield TAVO190191 (Table 2) in which the heterodimeric A chain and B chain of IGF2 are fused to the N-terminus of heavy chain and light chain of anti-TNFα antibody via the same MMP2/9 protease-cleavable linker sequence set forth as SEQ ID NO: 30. As another non-limiting example, the disclosure provides a shielded biologic by TavoPRECISE-Shield TAVO243244 (Table 2) in which the heterodimeric A chain and B chain of IGF2 are fused to the N-terminus of heavy chain and light chain of anti-HER2 antibody Trastuzumab via the same uPA protease-cleavable linker sequence set forth as SEQ ID NO: 31. The employment of four protease-cleavable linker sequences with substrate sequence cleavable by the same type of protease maintains the specificity of shielded biologics by TavoPRECISE-Shield activation. In this design, only one certain type of protease can remove the masking domains and convert the shielded biologics by TavoPRECISE-Shield into an active antibody.

In some embodiments, the four protease-cleavable linker sequences linking the two IGF2-based masking domains and the two Fab domains in the shielded biologics by TavoPRECISE-Shield comprise different sequences with substrate sequences cleavable by different types of proteases.

In some instances, the protease-cleavable linker sequence between the IGF2 chain and antibody heavy chain is comprised of a substrate sequence cleavable by one type of protease while the protease-cleavable linker sequence between the IGF2 chain and antibody light chain comprises substrate sequence cleavable by a different type of protease.

As a non-limiting example, the disclosure provides a shielded biologic by TavoPRECISE-Shield TAVO190197 (Table 2) in which the heterodimeric A chain and B chain of IGF2 are fused to the N-terminus of heavy chain and light chain of an anti-TNFα antibody via different types of protease-cleavable linker sequences. The A chain of IGF2 is fused to the N-terminus of heavy chain of the anti-TNFα antibody via MMP2/9 protease-cleavable linker sequence set forth as SEQ ID NO: 30 while the B chain of IGF2 is fused to the N-terminus of light chain of the anti-TNFα antibody via MMP3 protease-cleavable linker sequence set forth as SEQ ID NO: 32.

As another non-limiting example, the disclosure provides a shielded biologic by TavoPRECISE-Shield TAVO224244 (Table 2) in which the heterodimeric A chain and B chain of IGF2 are fused to the N-terminus of the heavy chain and light chain of anti-HER2 antibody Trastuzumab via different protease-cleavable linker sequences. The A chain of IGF2 is fused to the N-terminus of the heavy chain of Trastuzumab via MMP2/9 protease-cleavable linker sequence set forth as SEQ ID NO: 30 while the B chain of IGF2 is fused to the N-terminus of the light chain of Trastuzumab via uPA protease-cleavable linker sequence set forth as SEQ ID NO: 31. In this configuration, in which the heavy chain and light chain of the antibody are linked to the IGF2 chains via two different types of protease-cleavable linkers, either type of the proteases may cleave apart one of the two linkers and make the masking domain “cleaved open” and the shielded biologics by TavoPRECISE-Shield are converted into an active biologics. This particular design of shielded biologics by TavoPRECISE-Shield allows the shielded biologics to be activated in a disease tissue in which at least one of the two types of proteases is present.

In some instances, the protease-cleavable linker sequence between the IGF2 chains and one Fab arm comprises a substrate sequence cleavable by one type of protease while the protease-cleavable linker sequence between the IGF2 chains and the other Fab arm of the same antibody comprises a substrate sequence cleavable by a different type of protease. As a non-limiting example, the disclosure provides an anti-VEGF shielded antibody TAVO259229x246 (Table 2) in which the heterodimeric A chain and B chain of IGF2 are fused to one Fab arm of anti-VEGF antibody Bevacizumab via MMP2/9 protease-cleavable linker sequence set forth as SEQ ID NO: 30 and fused to the other Fab arm of anti-VEGF antibody Bevacizumab via uPA protease-cleavable linker sequence set forth as SEQ ID NO: 31. This type of molecule can be generated by controlled Fab arm exchange (Labrijn, Meesters et al. 2013). In this configuration in which the two Fab arms of the same antibody are linked to the IGF2 chains via two different types of protease-cleavable linkers, either type of the proteases may only cleave off just one of the two IGF2-based masking domains while the presence of both types of proteases is needed to cleave off both masking domains. This particular design of shielded biologics by TavoPRECISE-Shield allows the shielded biologics by TavoPRECISE-Shield to be fully activated in a disease tissue in which both types of proteases need to be present while the presence of just one of them may only partially activate the shielded biologics by TavoPRECISE-Shield.

In some instances, the protease-cleavable linker sequence between the IGF2 chains and one Fab arm of a bispecific antibody comprises a substrate sequence cleavable by one type of protease while the protease-cleavable linker sequence between the IGF2 chains and the other Fab arm of the bispecific antibody comprises a substrate sequence cleavable by a different type of protease. As a non-limiting example, the disclosure provides a bispecific anti-HER2×VEGF shielded antibody TAVO243244x229 (Table 2) in which the heterodimeric A chain and B chain of IGF2 are fused to the Fab arm of anti-HER2 antibody Trastuzumab via uPA protease-cleavable linker sequence set forth as SEQ ID NO: 31 and the heterodimeric A chain and B chain of IGF2 are fused to the Fab arm of anti-VEGF antibody Bevacizumab via MMP2/9 protease-cleavable linker sequence set forth as SEQ ID NO: 30. This type of bispecific shielded biologics by TavoPRECISE-Shield can be generated by controlled Fab arm exchange (Labrijn, Meesters et al. 2013). In this configuration in which the two Fab arms of the bispecific antibody to two different targets are linked to the IGF2 chains via two different types of protease-cleavable linkers, one type of the proteases may only cleave off the IGF2-based masking domain from the Fab arm with corresponding protease-cleavable linker sequence while the presence of both types of proteases is needed to cleave off both masking domains. This particular design of shielded biologics by TavoPRECISE-Shield allows the selective activation of the bispecific shielded biologics by TavoPRECISE-Shield to just one particular target or both of the two targets depending on the presence of particular type of proteases in the disease tissues.

Proteases are enzymes that catalyze the hydrolytic cleavage of peptide bonds of target proteins. Depending on the catalytic mechanisms, they can be divided into five distinct classes: serine, cysteine, aspartic, metallo- and threonine proteases. Physiologically, proteases are involved in numerous important processes including protein turnover, nutrient digestion, fertilization, cell differentiation and growth, the immune response, and apoptosis. The activity of proteases is normally tightly controlled through multiple redundant mechanisms, including regulation of biosynthesis, activation of inactive precursors known as pro-enzymes or zymogens, and by the binding of endogenous inhibitors and cofactors. However, during pathological conditions including cancer, autoimmune diseases and chronic inflammation, the expression and activity of proteases can be significantly up-regulated and inappropriate proteolysis can have a major role in the development of these diseases. For example, multiple proteases, including metalloproteinases, serine and cysteine proteases have up-regulation in the cancer microenvironment and execution of diverse functions at different stages of malignant progression, including tumor angiogenesis, invasion, and metastasis.

Due to the significant difference in the expression levels of certain types of protease in the disease lesion sites and normal tissues, engineering protease substrate sites in the shielded biologics by TavoPRECISE-Shield in this disclosure allows the conversion of the shielded biologics by TavoPRECISE-Shield into an active biologics selectively in the disease lesion site by protease-mediated cleavage of masking domains (e.g., IGF2-based). The disclosure provides the protease-cleavable linker sequence comprising substrate peptide sequence cleavable by any types of proteases whose expression level or activity is significantly higher in disease lesion site, including cancer, inflammation, autoimmune, cardiovascular, neurodegenerative, and bacterial and viral infection diseases.

Matrix metallopeptidases (MMPs), also known as matrix metalloproteinases or matrixins, are metalloproteinases that are calcium-dependent zinc-containing endopeptidases. Collectively, these enzymes are capable of degrading all kinds of extracellular matrix proteins. MMPs play critical roles during tissue remodeling in normal physiological processes, such as embryonic development and reproduction, as well as in disease processes, such as arthritis, and tumor metastasis. MMPs have been reported as one of the main factors of cancer progression and metastasis formation. MMPs have also been reported as major proteases in the inflammation sites during autoimmune and chronical inflammation diseases. The disclosure provides protease-cleavable linker sequences comprising substrate peptide sequences cleavable by different types of matrix metallopeptidases whose expression level or activity is significantly higher in disease lesion site relative to normal tissues.

Among the family of matrix metalloproteinases (MMPs), MMP2 and MMP9 are up-regulated in many types of cancers, including breast, colorectal and lung cancers. Besides, the expression and activity of MMP2 and MMP9 also correlates to the progression of many autoimmune disorders and inflammatory diseases, including rheumatoid arthritis, psoriasis, multiple sclerosis, chronic obstructed pulmonary disease, inflammatory bowel disease and osteoporosis (Lin, Lu et al. 2020). The disclosure provides a protease-cleavable linker sequence comprising a substrate peptide sequence cleavable by MMP2 or a substrate peptide sequence cleavable by MMP9. As non-limiting examples, the disclosure provides MMP2 and MMP9 cleavable substrate peptide sequence set forth as SEQ ID NO: 33, 34, 35, 36, 37.

Among the family of matrix metalloproteinases (MMPs), MMP-3 plays a critical role in the pathogenesis of rheumatoid arthritis (RA). It is produced mainly by synoviocytes and is a key enzyme involved in matrix degradation in RA. Several studies have suggested that elevated levels of MMP-3 are correlated between the serum and synovium of RA patients, and further that serum MMP-3 is a predictor of the degree of joint destruction in RA. The level of MMP3 in synovium is several hundred-fold more than that of serum level. The disclosure provides a protease-cleavable linker sequence comprising a substrate peptide sequence cleaved by MMP3. As non-limiting examples, the disclosure provides a MMP3-cleavable substrate peptide sequence set forth as SEQ ID NO: 38.

The urokinase plasminogen activator (uPA) has been reported to be overexpressed in many types of cancer, especially breast cancer (Banys-Paluchowski, Witzel et al. 2019). uPA is a serine protease that can catalyze the conversion of plasminogen to plasmin which can degrade the basement membrane or extracellular matrix. The matrix degradation can facilitate tumor cells migration and invasion into the surrounding tissue. The disclosure provides a protease-cleavable linker sequence comprising a substrate peptide sequence cleavable by uPA. As non-limiting examples, the disclosure provides uPA-cleavable substrate peptide sequences set forth as SEQ ID NO: 39 and 40.

The protease-cleavable linker of the present disclosure can optionally include one or more linker peptides interposed between, e.g., IGF2 chains and protease substrate peptide sequence, and/or between protease substrate peptide sequence and antibody chains.

Suitable linker peptides (also referred to as “spacers”) can be readily selected and can be of any of a number of suitable lengths, from 1 amino acid to 30 amino acids (e.g., any specific integer between 1 and 30, or from 1 amino acid (e.g., Gly) to about 20 amino acids (e.g., 2-15, 3-12, 4-10, 5-9, 6-8, or 7-8 amino acids).

Exemplary linker peptides include glycine polymers (G)ₙ, glycine-serine polymers (including, for example, (GS)ₙ, (GSGGS)ₙ (SEQ ID NO: 84) and (GGGS)ₙ (SEQ ID NO: 85), where n is an integer of at least one, e.g., 1, 2, 3, 4, 5, 6, 7, 8, 9, 10, 11, 12, 13, 14, 15, 16, 17, 18, 19, 20), glycine-alanine polymers, alanine-serine polymers, alanine-proline, immunoglobulin isotype and subtype hinge that can comprise IgG1, IgG2, IgG3, IgG4, IgA, IgE, IgM, and other flexible linker peptides known in the art. Both Gly and Ser are relatively unstructured, and therefore can serve as a neutral tether between components.

In certain embodiments, the linker peptide is a Glycine polymer. Glycine accesses significantly more phi-psi space than even alanine and is much less restricted than residues with longer side chains (see Scheraga, Rev. Computational Chem. 11173-142 (1992)). Exemplary linker peptides can comprise amino acid sequences including, but not limited to, GGS, GGSG (SEQ ID NO: 86), GGSGG (SEQ ID NO: 87), GGGGS (SEQ ID NO: 88), GSGSG (SEQ ID NO: 89), GSGGG (SEQ ID NO: 90), GGGSG (SEQ ID NO: 91), GSSSG (SEQ ID NO: 92), and the like.

In certain embodiments, the linker peptide is a rigid linker (Chen, Zaro et al. 2013). Exemplary rigid linker peptides can comprise amino acid sequences including, but not limited to, proline-rich sequence, (XP)ₙ, with X designating any amino acid, preferably Ala, Lys, or Glu, where n is an integer of at least one, e.g., 1, 2, 3, 4, 5, 6, 7, 8, 9, 10, 11, 12, 13, 14, 15, 16, 17, 18, 19, 20). Exemplary rigid linker peptides can also comprise amino acid sequences including, but not limited to, alpha helix-forming linkers with the sequence of (EAAAK)ₙ (SEQ ID NO: 93), where n is an integer of at least one, e.g., 1, 2, 3, 4, 5, 6, 7, 8, 9, 10, 11, 12, 13, 14, 15, 16, 17, 18, 19, 20.

### Therapeutic Antibodies and Fragments Applicable for Shielded Biologics by TavoPRECISE-Shield

Increasing numbers of therapeutic antibodies have shown impressive clinical efficacies in many disease areas, including cancer, autoimmune diseases, inflammation disorders and neurological and metabolism diseases. However, in many cases the pathological drug targets recognized by these therapeutic antibodies may also have significant expression in normal tissues and play critical physiological roles. As a result, systemic administration of such a therapeutic antibody may lead to mechanism-based on-target toxicities due to the non-specific attack of the drug target in the normal tissue besides the same target in the disease sites by the therapeutic antibody. To address this safety problem, this disclosure provides shielded biologics by TavoPRECISE-Shield comprising masking domains (e.g., IGF2-based) fused to the Fab domains of therapeutic antibodies via protease-cleavable linkers. For example, in normal tissue, the antigen-binding capability of the therapeutic antibody is inhibited or blocked by the IGF2-based masking domains. In the disease site, the disease-associated proteases can recognize the protease-cleavable linker sequence and cleave off or cleave open the IGF2-based masking domain, thus turning on the activity of the therapeutic antibody from the shielded biologic by TavoPRECISE-Shield. This shielded biologic by TavoPRECISE-Shield approach helps to improve the site of action of therapeutic biologics for them to engage targets mainly at the disease site while sparing those targets in normal healthy tissue.

In some embodiments, the disclosure provides therapeutic antibodies and fragments to which IGF2-based masking domains are attached to their Fab domains via protease-cleavage linker sequences to make shielded biologics by TavoPRECISE-Shield. The targets of such therapeutic antibodies have differential expression levels in pathological sites and normal tissues. The shielded biologics by TavoPRECISE-Shield remain largely inactive in normal tissue due to the inhibitory effects of the IGF2-based masking domain on the activities of the therapeutic antibodies. The IGF2-based masking domains are cleaved off by proteases in the disease site and the shielded biologics by TavoPRECISE-Shield are converted to active therapeutic biologics.

The therapeutic antibodies and fragments applicable for the shielded biologics by TavoPRECISE-Shield of the present disclosure encompass full length antibodies comprising two heavy chains and two light chains. The antibodies can be human or humanized antibodies. Humanized antibodies include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody. Exemplary human or humanized antibodies include IgG, IgM, IgE, IgA, and IgD antibodies. The present antibodies can be of any class (IgG, IgM, IgE, IgA, IgD, etc.) or isotype. For example, a human antibody can comprise an IgG Fc domain, such as at least one of isotypes, IgG1, IgG2, IgG3 and IgG4.

In some embodiments of the disclosure, the therapeutic antibody is an anti-TNFα antibody. TNFα is a pathological proinflammation factor critical for the development of many autoimmune diseases and anti-TNFα antibodies have become major therapies for rheumatoid arthritis (RA), Crohn's disease and many more types of autoimmune diseases. However, physiologically TNFα is an immune modulator critical for host defense, and blockade of TNFα with anti-TNFαbiologics drugs may lead to serious on-target toxicities including severe infections and elevated risk of malignancy (Fiorentino, Ho et al. 2017, Fernandez-Ruiz and Aguado 2018). Long term administration of anti-TNFα biologic drugs pose a great risk factor for patients. The conversion of anti-TNFα antibodies into shielded biologics by TavoPRECISE-Shield with the fusion of masking domains (e.g., IGF2-based) may increase the safety profile and therapeutic window of anti-TNFα antibodies.

As a non-limiting example, the disclosure provides an anti-TNFα shielded biologic by TavoPRECISE-Shield in which IGF2-based masking domains are fused to anti-TNFαantibody Adalimumab via MMP2/9 protease-cleavable linker sequences, comprising a heavy chain polypeptide with sequence set forth as SEQ ID NO. 41, comprising, in an N-terminus to C-terminus orientation, the A chain of IGF2 with V43L mutation, MMP2/9 protease-cleavable linker sequence and Adalimumab heavy chain sequence, and a light chain polypeptide with sequence set forth as SEQ ID NO. 42, comprising, in an N-terminus to C-terminus orientation, the B chain of IGF2 with Y27A mutation, MMP2/9 protease-cleavable linker sequence and Adalimumab light chain sequence.

As a non-limiting example, the disclosure provides an anti-TNFα shielded biologic by TavoPRECISE-Shield in which IGF2-based masking domains are fused to anti-TNFα antibody Infliximab via MMP3 protease-cleavable linker sequences, comprising a heavy chain polypeptide with sequence set forth as SEQ ID NO. 43, comprising, in an N-terminus to C-terminus orientation, the A chain of IGF2 with V43L mutation, MMP3 protease-cleavable linker sequence and Infliximab heavy chain sequence, and a light chain polypeptide with sequence set forth as SEQ ID NO.44, comprising, in an N-terminus to C-terminus orientation, the B chain of IGF2 with Y27A mutation, MMP3 protease-cleavable linker sequence and Infliximab light chain sequence.

In some embodiments of the disclosure, the therapeutic antibody is an anti-IL-1β antibody. Blocking IL-1β using anti-IL-1β antibody Canakinumab has shown clinical efficacy in several severe auto-inflammatory diseases, including cryopyrin-associated periodic syndromes (CAPS). However, there is a warning for potential increased risk of serious infections due to IL-1 blockade associated with Canakinumab. As a non-limiting example, the disclosure provides an anti-IL-1β shielded biologic by TavoPRECISE-Shield in which IGF2-based masking domains are fused to anti-IL-1β antibody Canakinumab via MMP2/9 protease-cleavable linker sequences, comprising a heavy chain polypeptide with sequence set forth as SEQ ID NO. 45, comprising, in an N-terminus to C-terminus orientation, the A chain of IGF2 with V43L mutation, MMP2/9 protease-cleavable linker sequence and Canakinumab heavy chain sequence, and a light chain polypeptide with sequence set forth as SEQ ID NO.46, comprising, in an N-terminus to C-terminus orientation, the B chain of IGF2 with Y27A mutation, MMP2/9 protease-cleavable linker sequence and Canakinumab light chain sequence.

In some embodiments of the disclosure, the therapeutic antibody is an anti-HER2 antibody. Trastuzumab, an anti-HER2 antibody, was approved to treat HER2 positive breast cancer. However, administration of Trastuzumab is associated with cardiotoxicity due to the presence of HER2 in the heart. As a non-limiting example, the disclosure provides an anti-HER2 shielded biologic by TavoPRECISE-Shield in which IGF2-based masking domains are fused to anti-HER2 antibody Trastuzumab via MMP2/9 protease-cleavable linker sequences, comprising a heavy chain polypeptide EAC224 with sequence set forth as SEQ ID NO. 9, comprising, in an N-terminus to C-terminus orientation, the A chain of IGF2, MMP2/9 protease-cleavable linker sequence and Trastuzumab heavy chain sequence, and a light chain polypeptide EAC225 with sequence set forth as SEQ ID NO. 10, comprising, in an N-terminus to C-terminus orientation, the B chain of IGF2, MMP2/9 protease-cleavable linker sequence and Trastuzumab light chain sequence.

In some embodiments of the disclosure, the therapeutic antibody is an anti-VEGF antibody. Bevacizumab, an anti-VEGF antibody, was approved to treat various cancers, including colorectal, lung, breast, renal, brain and ovarian cancers. It is also approved for many eye diseases associated with VEGF-driven blood vessel growth in the eye. However, administration of bevacizumab is associated with the worsening of coronary and peripheral artery diseases, including hypertension and heightened risk of bleeding. As a non-limiting example, the disclosure provides an anti-VEGF shielded biologic by TavoPRECISE-Shield in which IGF2-based masking domains are fused to anti-VEGF antibody Bevacizumab via uPA protease-cleavable linker sequences, comprising a heavy chain polypeptide EAC245 with sequence set forth as SEQ ID NO. 15, comprising, in an N-terminus to C-terminus orientation, the A chain of IGF2, uPA protease-cleavable linker sequence and Bevacizumab heavy chain sequence, and a light chain polypeptide EAC246 with sequence set forth as SEQ ID NO. 16, comprising, in an N-terminus to C-terminus orientation, the B chain of IGF2, uPA protease-cleavable linker sequence and Bevacizumab light chain sequence.

In some embodiments of the disclosure, the therapeutic antibody is an anti-EGFR antibody. Anti-EGFR antibodies were approved to treat various cancers, including colorectal, lung, and head and neck cancers. However, administration of anti-EGFR antibodies is associated with skin toxicity such as skin rash due to the physiological role of EGFR in the epidermis. As a non-limiting example, the disclosure provides an anti-EGFR shielded biologic by TavoPRECISE-Shield in which IGF2-based masking domains are fused to anti-EGFR antibody Cetuximab via MMP2/9 protease-cleavable linker sequences, comprising a heavy chain polypeptide with sequence set forth as SEQ ID NO. 47, comprising, in an N-terminus to C-terminus orientation, the A chain of IGF2 with V43L mutation, MMP2/9 protease-cleavable linker sequence and Cetuximab heavy chain sequence, and a light chain polypeptide with sequence set forth as SEQ ID NO. 48, comprising, in an N-terminus to C-terminus orientation, the B chain of IGF2 with Y27A mutation, uPA protease-cleavable linker sequence and Cetuximab light chain sequence.

In some embodiments of the disclosure, the therapeutic antibody is an anti-CTLA-4 antibody. Anti-CTLA-4 antibodies were approved, or in clinical trial, to treat various cancers, including melanoma, lung, bladder and prostate cancers. However, administration of anti-CTLA-4 antibodies is associated with various autoimmune side effects, including rash, colitis/diarrhea, dermatitis, hepatitis, and endocrinopathies, likely due to breaking immune tolerance upon CTLA-4 blockade (Di Giacomo, Biagioli et al. 2010). As a non-limiting example, the disclosure provides an anti-CTLA-4 shielded biologic by TavoPRECISE-Shield in which IGF2-based masking domains are fused to anti-CTLA-4 antibody Ipilimumab via uPA protease-cleavable linker sequences, comprising a heavy chain polypeptide with sequence set forth as SEQ ID NO. 49, comprising, in an N-terminus to C-terminus orientation, the A chain of IGF2 with V43L mutation, uPA protease-cleavable linker sequence and Ipilimumab heavy chain sequence, and a light chain polypeptide with sequence set forth as SEQ ID NO. 50, comprising, in an N-terminus to C-terminus orientation, the B chain of IGF2 with Y27A mutation, uPA protease-cleavable linker sequence and Ipilimumab light chain sequence.

In some embodiments of the disclosure, the therapeutic antibody is an anti-CD3ε antibody. Anti-CD3ε antibodies were approved as an immunosuppressant drug given to reduce acute rejection in patients with organ transplants. In more recent years, the Fv part of anti-CD3ε antibody is engineered as part of T cell redirection molecule to engage T cells to kill conjugated tumor cells. However, non-specific T cell activation by anti-CD3ε antibodies is associated with various autoimmune side effects due to breaking immune tolerance upon T cell activation. As a non-limiting example, the disclosure provides a masked anti-CD3ε molecule in which IGF2-based masking domains are fused to the Fab of anti-CD3ε antibody Muromonab via MMP2/9 protease-cleavable linker sequences, comprising a heavy chain polypeptide with sequence set forth as SEQ ID NO. 51, comprising, in an N-terminus to C-terminus orientation, the A chain of IGF2 with V43L mutation, MMP2/9 protease-cleavable linker sequence and Muromonab V_{H} sequence, and light chain polypeptide with sequence set forth as SEQ ID NO. 52, comprising, in an N-terminus to C-terminus orientation, the B chain of IGF2 with Y27A mutation, MMP2/9 protease-cleavable linker sequence and Muromonab V_{L} sequence.

In some embodiments of the disclosure, the therapeutic antibody is an antibody against a4-integrin, CD20, CD11a, CD52, RANK-L, PD-1, PD-L1, CD24, CD47, CD166, or CD71. All these drug targets have high expression in disease sites but also have expression in normal tissues. The disclosure provides shielded biologics by TavoPRECISE-Shield for these therapeutic antibodies in which IGF2-based masking domains are fused to these therapeutic antibodies via appropriate protease-cleavable linker sequences, comprising heavy chain polypeptide comprising, in an N-terminus to C-terminus orientation, the A chain of IGF2 with V43L mutation, protease-cleavable linker sequence and an antibody heavy chain sequence, and light chain polypeptide comprising, in an N-terminus to C-terminus orientation, the B chain of IGF2 with Y27A mutation, protease-cleavable linker sequence and an antibody light chain sequence.

The therapeutic antibodies and fragments applicable for the shielded biologics by TavoPRECISE-Shield of the present disclosure encompass bispecific antibody.

As a non-limiting example, the disclosure provides a bispecific shielded biologic by TavoPRECISE-Shield TAVO190191x212213 in which the IGF2-based masking domains are fused to the Fab arm of an anti-TNFα antibody and to the Fab arm of an anti-IL-1 antibody via MMP2/9 protease-cleavable linker sequences. The anti-TNFα arm comprises a heavy chain polypeptide EAC190 with sequence set forth as SEQ ID NO. 1, comprising, in an N-terminus to C-terminus orientation, the A chain of IGF2, MMP2/9 protease-cleavable linker sequence and anti-TNFα antibody heavy chain sequence, and light chain polypeptide EAC191 with sequence set forth as SEQ ID NO. 2, comprising, in an N-terminus to C-terminus orientation, the B chain of IGF2, MMP2/9 protease-cleavable linker sequence and anti-TNFα antibody light chain sequence. The anti-IL-1 arm comprises a heavy chain polypeptide EAC212 with sequence set forth as SEQ ID NO. 5, comprising, in an N-terminus to C-terminus orientation, the A chain of IGF2, MMP2/9 protease-cleavable linker sequence and anti-IL-1β antibody heavy chain sequence, and light chain polypeptide EAC213 with sequence set forth as SEQ ID NO. 6, comprising, in an N-terminus to C-terminus orientation, the B chain of IGF2, MMP2/9 protease-cleavable linker sequence and anti-IL-1 antibody light chain sequence. This type of bispecific shielded biologics by TavoPRECISE-Shield can be generated by controlled Fab arm exchange (Labrijn, Meesters et al. 2013).

As a non-limiting example, the disclosure provides a bispecific shielded biologic by TavoPRECISE-Shield TAVO224225x259229 in which the IGF2-based masking domains are fused to the Fab arm of anti-HER2 antibody Trastuzumab and to the Fab arm of anti-VEGF antibody Bevacizumab via MMP2/9 protease-cleavable linker sequences. The anti-HER2 arm comprises a heavy chain polypeptide EAC224 with sequence set forth as SEQ ID NO. 9, comprising, in an N-terminus to C-terminus orientation, the A chain of IGF2, MMP2/9 protease-cleavable linker sequence and Trastuzumab heavy chain sequence, and light chain polypeptide EAC225 with sequence set forth as SEQ ID NO. 10, comprising, in an N-terminus to C-terminus orientation, the B chain of IGF2, MMP2/9 protease-cleavable linker sequence and Trastuzumab light chain sequence. The anti-VEGF arm comprises a heavy chain polypeptide EAC259 with sequence set forth as SEQ ID NO. 13, comprising, in an N-terminus to C-terminus orientation, the A chain of IGF2, MMP2/9 protease-cleavable linker sequence and Bevacizumab heavy chain sequence, and light chain polypeptide EAC229 with sequence set forth as SEQ ID NO. 14, comprising, in an N-terminus to C-terminus orientation, the B chain of IGF2, MMP2/9 protease-cleavable linker sequence and Bevacizumab light chain sequence. This type of bispecific shielded biologics by TavoPRECISE-Shield can be generated by controlled Fab arm exchange (Labrijn, Meesters et al. 2013).

The therapeutic antibodies and fragments applicable for the shielded biologics by TavoPRECISE-Shield of the present disclosure encompass antigen-binding fragments that retain sufficient ability to specifically bind to therapeutic drug targets. The binding fragments as used herein may include any 3 or more contiguous amino acids (e.g., 4 or more, 5 or more 6 or more, 8 or more, or even 10 or more contiguous amino acids) of the antibody and encompasses Fab, Fab′, F(ab′)2, and F(v) fragments, or the individual light or heavy chain variable regions or portions thereof. These fragments lack the Fc fragment of an intact antibody, clear more rapidly from the circulation, and can have less non-specific tissue binding than an intact antibody. These fragments can be produced from intact antibodies using well known methods, for example by proteolytic cleavage with enzymes such as papain (to produce Fab fragments) or pepsin (to produce F(ab′)2 fragments).

The therapeutic antibodies and fragments applicable for the shielded biologics by TavoPRECISE-Shield of the present disclosure encompass antigen-binding fragments fused to non-antibody-based scaffolds include, e.g., albumin, an XTEN (extended recombinant) polypeptide, transferrin, an F_{c} receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly (SEQ ID NO: 94)), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

### IgG Fc of Shielded Biologics by TavoPRECISE-Shield

In some embodiments, a shielded biologic by TavoPRECISE-Shield with IGF2-based masking domain as disclosed herein may comprise a modified Fc region, wherein the modified Fc region comprises at least one amino acid modification relative to a native Fc region. In some embodiments, a present shielded biologic by TavoPRECISE-Shield with IGF2-based masking domain is provided with a modified Fc region where a naturally occurring Fc region is modified to extend the half-life of the antibody when compared to the parental native antibody in a biological environment, for example, the serum half-life or a half-life measured by an in vitro assay. Exemplary mutations that may be made singularly or in combination include T250Q, M252Y, I253A, S254T, T256E, P257I, T307A, D376V, E380A, M428L, H433K, N434S, N434A, N434H, N434F, H435A and H435R mutations.

In certain embodiments, the extension of half-life can be realized by engineering the M252Y/S254T/T256E mutations in IgG1 F_{c} residue, numbering according to the EU Index (Dall'Acqua, Kiener et al. 2006).

In certain embodiments, the extension of half-life can also be realized by engineering the M428L/N434S mutations in IgG₁ F_{c} (Zalevsky, Chamberlain et al. 2010).

In certain embodiments, the extension of half-life can also be realized by engineering the T250Q/M428L mutations in IgG₁ F_{c} (Hinton, Xiong et al. 2006).

In certain embodiments, the extension of half-life can also be realized by engineering the N434A mutations in IgG₁ F_{c} (Shields, Namenuk et al. 2001).

In certain embodiments, the extension of half-life can also be realized by engineering the T307A/E380A/N434A mutations in IgG₁ F_{c} (Petkova, Akilesh et al. 2006).

The effect of F_{c} engineering on the extension of antibody half-life can be evaluated in PK studies in mice, relative to antibodies with native IgG F_{c}.

In some embodiments, the present shielded biologics by TavoPRECISE-Shield with IGF2-based masking domain are provided with a modified F_{c} region where a naturally-occurring F_{c} region is modified to enhance the antibody resistance to proteolytic degradation by a protease that cleaves the wild-type antibody between or at residues 222-237 (EU numbering).

In certain embodiments, the resistance to proteolytic degradation can be realized by engineering E233P/L234A/L235A mutations in the hinge region with G236 deleted when compared to a parental native antibody, residue numbering according to the EU Index (Kinder, Greenplate et al. 2013).

In instances where effector functionality is not desired, the antibodies of the disclosure may further be engineered to introduce at least one mutation in the antibody F_{c} that reduces binding of the antibody to an activating F_{c}γ receptor (F_{c}γR) and/or reduces F_{c} effector functions such as C1q binding, complement dependent cytotoxicity (CDC), antibody-dependent cell-mediated cytotoxicity (ADCC) or phagocytosis (ADCP).

F_{c} positions that may be mutated to reduce binding of the antibody to the activating F_{c}γR and subsequently reduce effector functions, including those described for example in (Xu, Alegre et al. 2000) (Vafa, Gilliland et al. 2014) (Bolt, Routledge et al. 1993) (Chu, Vostiar et al. 2008) (Shields, Namenuk et al. 2001). F_{c} mutations with minimal ADCC, ADCP, CDC, F_{c} mediated cellular activation have been described also as sigma mutations for IgG₁, IgG₂ and IgG₄ (Tam, McCarthy et al. 2017). Exemplary mutations that may be made singularly or in combination include K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S mutations on IgG₁, IgG₂, IgG₃ or IgG₄.

Exemplary combination mutations that may be made to reduced ADCC include L234A/L235A on IgG₁, V234A/G237A/P238S/H268A/V309L/A330S/P331S on IgG₂, F234A/L235A on IgG₄, S228P/F234A/L235A on IgG₄, N297A on IgG₁, IgG₂, IgG₃ or IgG₄, V234A/G237A on IgG₂, K214T/E233P/L234V/L235A/G236-deleted/A327G/P331A/D365E/L358M on IgG₁, H268Q/V309L/A330S/P331S on IgG₂, S267E/L328F on IgG₁, L234F/L235E/D265A on IgG₁, L234A/L235A/G237A/P238S/H268A/A330S/P331S on IgG₁, S228P/F234A/L235A/G237A/P238S on IgG₄, and S228P/F234A/L235A/G236-deleted/G237A/P238S on IgG₄. Hybrid IgG_{2/4} F_{c} domains may also be used, such as F_{c} with residues 117-260 from IgG₂ and residues 261-447 from IgG₄.

In some embodiments, the present shielded biologics by TavoPRECISE-Shield with IGF2-based masking domain are provided with a modified F_{c} region where a naturally-occurring F_{c} region is modified to facilitate the generation of a bispecific antibody by F_{c} heterodimerization.

In certain embodiments, the Fc heterodimerization can be realized by engineering F405L and K409R mutations on two parental antibodies and the generation of a bispecific antibody in a process known as Fab arm exchange (Labrijn, Meesters et al. 2014).

In certain embodiments, the Fc heterodimerization can also be realized by Fc mutations to facilitate Knob-in-Hole strategy (see, e.g., Intl. Publ. No. WO 2006/028936). An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole include: T366Y/F405A, T366W/F405W, F405W/Y407A, T394W/Y407T, T394S/Y407A, T366W/T394S, F405W/T394S and T366W/T366S/L368A/Y407V.

In certain embodiments, the Fc heterodimerization can also be realized by Fc mutations to facilitate the electrostatically-matched interactions strategy (Gunasekaran, Pentony et al. 2010). Mutations can be engineered to generate positively charged residues at one Fc domain and negatively charged residues at the other Fc domain as described in US Patent Publ. No. US2010/0015133; US Patent Publ. No. US2009/0182127; US Patent Publ. No. US2010/028637 or US Patent Publ. No. US2011/0123532. Heavy chain heterodimerization can be formed by electrostatically-matched interactions between two mutated Fc.

In some embodiments, the present shielded biologics by TavoPRECISE-Shield with IGF2-based masking domain are provided with a modified F_{c} region where a naturally occurring F_{c} region is modified to facilitate the multimerization of the antibody upon interaction with cell surface receptors, although such engineered antibody exist as monomer in solution. The F_{c} mutations that facilitate antibody multimerization include, but not limited to, E345R mutation, E430G mutation, E345R/E430G mutations, E345R/E430G/Y440R mutations as described in (Diebolder, Beurskens et al. 2014). Such mutations may also include, but not limited to, T437R mutation, T437R/K248E mutations, T437R/K338A mutations as described in (Zhang, Armstrong et al. 2017).

Antibodies of the disclosure further comprising conservative modifications are within the scope of the disclosure. “Conservative modifications” refer to amino acid modifications that do not significantly affect or alter the binding characteristics of the antibody containing the amino acid sequences. Conservative modifications include amino acid substitutions, additions, and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

The antibodies of the disclosure may be post-translationally modified by processes such as glycosylation, isomerization, deglycosylation or non-naturally occurring covalent modification, such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function.

Antibodies of the disclosure may be modified to improve stability, selectivity, cross-reactivity, affinity, immunogenicity or other desirable biological or biophysical property which are within the scope of the disclosure. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn and Pluckthun 2001). Potential structure destabilizing residues may be identified based upon the crystal structure of the antibody or by molecular modelling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of the disclosure may have amino acid substitutions in the F_{c} region that improve manufacturing and drug stability. An example for IgG₁ is H224S (or H224Q) in the hinge 221-DKTHTC-226 (Eu numbering) which blocks radically induced cleavage; and for IgG₄, the S228P mutation blocks half-antibody exchange.

### Leader Sequences

In certain embodiments, a leader peptide is chosen to drive the secretion of a shielded biologic by TavoPRECISE-Shield with IGF2-based masking domain described in this disclosure into the cell culture supernatant as a secreted protein. Any leader peptide for any known secreted proteins/peptides can be used.

As used herein, a “leader peptide” or “signal peptide” includes a short peptide, usually 16-30 amino acids in length, that is present at the N-terminus of most of the newly synthesized proteins that are destined towards the secretory pathway. Although leader peptides are extremely heterogeneous in sequence, and many prokaryotic and eukaryotic leader peptides are functionally interchangeable even between different species, the efficiency of protein secretion may be strongly determined by the sequence of the leader/signal peptide.

In certain embodiments, the leader peptide that is from a protein residing either inside certain organelles (such as the endoplasmic reticulum, Golgi or endosomes), secreted from the cell, or inserted into most cellular membranes may be used.

In certain embodiments, the leader peptide is from a eukaryotic protein.

In certain embodiments, the leader peptide is from a secreted protein, e.g., a protein secreted outside a cell.

In certain embodiments, the leader peptide is from a transmembrane protein.

In certain embodiments, the leader peptide contains a stretch of amino acids that is recognized and cleaved by a signal peptidase.

In certain embodiments, the leader peptide does not contain a cleavage recognition sequence of a signal peptidase.

In certain embodiments, the leader peptide is a signal peptide for tissue plasminogen activator (tPA), herpes simplex virus glycoprotein D (HSV gD), a growth hormone, a cytokine, a lipoprotein export signal, CD2, CD3δ, CD3ε, CD3γ, CD3ζ, CD4, CD8α, CD19, CD28, 4-1BB or GM-CSFR, or S. cerevisiae mating factor α-1 signal peptide.

In some embodiments, a leader sequence as described herein may be a mammalian CD4 or CD8 leader sequence, including but not limited to, e.g., a human CD4 or CD8 leader sequence, a non-human primate CD4 or CD8 leader sequence, a rodent CD4 or CD8 leader sequence, and the like. In some embodiments, a CD4 or CD8 leader comprises an amino acid sequence having at least 75%, at least 80%, at least 85%, at least 90%, at least 95%, at least 98%, at least 99%, or 100%, amino acid sequence identity with the human CD4 or CD8 leader sequences.

### Additional Polypeptides

Suitable additional polypeptides include epitope tags and affinity domains. The one or more additional polypeptide can be included at the N-terminus, at the C-terminus, or internally of the shielded biologics by TavoPRECISE-Shield with a masking domain (e.g., IGF2-based) described in this disclosure.

Suitable epitope tags include, but are not limited to, hemagglutinin HA e.g., YPYDVPDYA (SEQ ID NO: 95); FLAG e.g., DYKDDDDK (SEQ ID NO: 96); c-myc e.g., EQKLISEEDL (SEQ ID NO: 97), and the like.

Affinity domains include peptide sequences that can interact with a binding partner, e.g., one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel Sepharose or other immobilized metal affinity chromatography resins. Exemplary affinity domains include His5 (HHHHH (SEQ ID NO: 98)), HisX6 (HHHHHH (SEQ ID NO: 99)), C-myc (EQKLISEEDL (SEQ ID NO: 97)), Flag (DYKDDDDK (SEQ ID NO: 96)), Strep Tag (WSHPQFEK (SEQ ID NO: 100)), hemagglutinin, e.g., HA Tag (YPYDVPDYA (SEQ ID NO: 95)), glutathione-S-transferase (GST), thioredoxin, cellulose binding domain, RYIRS (SEQ ID NO: 101), Phe-His-His-Thr (SEQ ID NO: 102), chitin binding domain, S-peptide, T7 peptide, SH2 domain, C-end RNA tag, WEAAAREACCRECCARA (SEQ ID NO: 83), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

In some embodiments, affinity domains include peptide sequences that can interact with a binding partner in the disease site. The shielded biologics by TavoPRECISE-Shield with one or more such affinity domains to targets in disease sites may facilitate the accumulation of the shielded biologics by TavoPRECISE-Shield in disease sites with higher concentration relative to normal tissues. Such selective accumulation or homing of the shielded biologics by TavoPRECISE-Shield with a masking domain (e.g., IGF2-based) described in this disclosure may help to increase the effective amounts of therapeutic antibody drug selectively in disease sites.

Shielded biologics by TavoPRECISE-Shield with a masking domain (e.g., IGF2-based) described in this disclosure can include peptide sequences that can facilitate the expression and secretion of the shielded biologics by TavoPRECISE-Shield from the host cells that express them, or the proper folding of the masking domain, or the accessibility of the protease-cleavable linker, or the proper folding of the therapeutic antibody. The peptide sequence described herein can be included at the N-terminus, at the C-terminus, or internally of the shielded biologics by TavoPRECISE-Shield described in this disclosure.

Shielded biologics by TavoPRECISE-Shield with a masking domain (e.g., IGF2-based) described in this disclosure can include one or more non-polypeptide moieties covalently linked to the shielded biologics by TavoPRECISE-Shield. Suitable non-polypeptide moieties include, e.g., biocompatible fatty acids and derivatives thereof; Hydroxy Alkyl Starch (HAS), e.g., Hydroxy Ethyl Starch (HES); poly (ethylene glycol); hyaluronic acid (HA); heparosan polymers (HEP); phosphorylcholine-based polymers; dextran; poly-sialic acids (PSA); and the like. In some cases, the non-polypeptide moiety increases the in vivo half-life of the shielded biologics by TavoPRECISE-Shield with IGF2-based masking domain described in this disclosure, compared to a control shielded biologic by TavoPRECISE-Shield that does not comprise the non-polypeptide moiety.

In some cases, the shielded biologics by TavoPRECISE-Shield with a masking domain (e.g., IGF2-based) described in this disclosure include a detectable label. Suitable detectable labels include radioisotopes such as ¹²³I (iodine), ¹⁸F (fluorine), ⁹⁹Tc (technetium), (indium), ⁶⁷Ga (gallium), radioactive Gd isotopes (¹⁵³Gd); contrast agents such as gadolinium (Gd), dysprosium, and iron; an enzyme which generates a detectable product (e.g., luciferase, (3-galactosidase, horse radish peroxidase, alkaline phosphatase, and the like); a fluorescent protein; a chromogenic protein, dye (e.g., fluorescein isothiocyanate, rhodamine, phycoerythrin, and the like); fluorescence emitting metals, e.g., ¹⁵²Eu, or others of the lanthanide series; chemiluminescent compounds, e.g., luminol, isoluminol, acridinium salts, and the like; bioluminescent compounds; and the like.

### Expression and Purification of Shielded Biologics by TavoPRECISE-Shield

A shielded biologic by TavoPRECISE-Shield with a masking domain (e.g., IGF2-based) of the present disclosure can be encoded by a single nucleic acid (e.g., a single nucleic acid comprising nucleotide sequences that encode the light and heavy chain polypeptides of a shielded biologic by TavoPRECISE-Shield), or by two or more separate nucleic acids, each of which encode a different part of the shielded biologic by TavoPRECISE-Shield.

As a non-limiting example, the disclosure provides nucleic acid sequence as SEQ ID NO: 53 which encodes the heavy chain polypeptide EAC190 as SEQ ID NO: 1, and nucleic acid sequence as SEQ ID NO: 54 which encodes the light chain polypeptide EAC191 as SEQ ID NO: 2, of anti-TNFα shielded biologic by TavoPRECISE-Shield TAVO190191 (Table 2).

The nucleic acids described herein can be inserted into vectors, e.g., nucleic acid expression vectors and/or targeting vectors. Such vectors can be used in various ways, e.g., for the expression of shielded biologics by TavoPRECISE-Shield with IGF2-based masking domain described herein in a cell or transgenic animal. Vectors are typically selected to be functional in the host cell in which the vector will be used. A nucleic acid molecule encoding a shielded biologic by TavoPRECISE-Shield with IGF2-based masking domain described herein may be amplified/expressed in prokaryotic, yeast, insect (baculovirus systems) and/or eukaryotic host cells. Selection of the host cell will depend in part on whether a shielded biologic by TavoPRECISE-Shield with IGF2-based masking domain described herein is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

In most cases, a leader or signal sequence is engineered at the N-terminus of the shielded biologics by TavoPRECISE-Shield with IGF2-based masking domain described herein to guide its secretion. The secretion of the shielded biologics by TavoPRECISE-Shield with IGF2-based masking domain from a host cell will result in the removal of the signal peptide from the antibody. Thus, the mature antibody will lack any leader or signal sequence. In some cases, such as where glycosylation is desired in a eukaryotic host cell expression system, one may manipulate the various presequences to improve glycosylation or yield. For example, one may alter the peptidase cleavage site of a signal peptide, or add prosequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure so as to produce a polypeptide encoded thereby. To express the shielded biologics by TavoPRECISE-Shield with IGF2-based masking domain described herein, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector as disclosed herein into the cell, the cell is cultured under conditions suitable for expression of the encoded sequence. The shielded biologics by TavoPRECISE-Shield then can be isolated from the cell.

In certain embodiments, two or more vectors that together encode a shielded biologic by TavoPRECISE-Shield with IGF2-based masking domain described herein, can be introduced into the cell.

Purification of the shielded biologic by TavoPRECISE-Shield with a masking domain (e.g., IGF2-based) described herein, which has been secreted into the cell media, can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising a F_{c} region may be purified by affinity chromatography with Protein A, which selectively binds the F_{c} region.

Modified forms of the shielded biologics by TavoPRECISE-Shield with a masking domain (e.g., IGF2-based) may be prepared with affinity tags, such as hexahistidine (SEQ ID NO: 99) or other small peptide such as FLAG (Eastman Kodak Co., New Haven, Conn.) or myc (Invitrogen) at either its carboxyl or amino terminus and purified by a one-step affinity column. For example, polyhistidine binds with great affinity and specificity to nickel, thus an affinity column of nickel (such as the Qiagen® nickel columns) can be used for purification of polyhistidine-tagged selective binding agents. In some instances, more than one purification step may be employed.

### Effects of Masking Domain on Binding and Functional Activity of Therapeutic Antibody

A masking domain (e.g., IGF2-based) when fused to a therapeutic antibody can inhibit or block the capability of the antibody in binding to its antigen. The masking domain may reduce the maximum binding capacity of the shielded biologic by TavoPRECISE-Shield in binding to its antigen. The masking domain may also reduce the binding affinity of the shielded biologic by TavoPRECISE-Shield in binding to its antigen.

When the masking domain is cleaved off by protease, the shielded biologic by TavoPRECISE-Shield is converted to an active antibody with the restoration of the capability of the antibody in binding to its antigen. The removal of the IGF2-based masking domain from the shielded biologics by TavoPRECISE-Shield can be realized by in vitro protease cutting assay using recombinant or purified protease. The removal of IGF2-based masking domain from the shielded biologics by TavoPRECISE-Shield can also be realized in vivo by proteases overexpressed in disease sites. The removal of IGF2-based masking domain can be assessed by comparing the molecular weight of heavy chain and light chain of the shielded biologics by TavoPRECISE-Shield with the IGF2-based masking domain and active antibodies with the masking domain cleaved off by SDS-PAGE analysis. The degree of removal of IGF2-based masking domain can be assessed by the intensity of the protein bands on protein gel.

In vitro and cell-based assays are well described in the art for use in determining the shielded biologics by TavoPRECISE-Shield, active biologics (i.e., the therapeutic biologics not in shielded form) and converted biologics after protease cleavage in binding to its antigen. For example, the binding of the antibody may be determined by ELISA by immobilizing recombinant or purified antigen, sequestering the antibody with the immobilized antigen and determining the amount of bound antibody. This can also be performed using a Biacore® instrument for kinetic analysis of binding interactions. For cell-based binding assay, the binding of the antibody may be determined by flow cytometry by incubating the antibody with cells expressing antigens on cell surface and determining the amount of antibody bound to cell surface antigen.

In some embodiments, the presence of IGF2-based masking domain may inhibit the binding of the therapeutic biologic to its antigen by at least 10 percent, alternatively at least 20 percent, alternatively at least 30 percent, alternatively at least 40 percent, alternatively at least 50 percent, alternatively at least 60 percent, alternatively at least 70 percent, alternatively at least 80 percent, alternatively at least 90 percent, or alternatively 100 percent. The removal of IGF2-based masking domain from the shielded biologic by TavoPRECISE-Shield by protease-mediated cleavage can restore the capacity of the converted antibody in antigen binding by at least 10 percent, alternatively at least 20 percent, alternatively at least 30 percent, alternatively at least 40 percent, alternatively at least 50 percent, alternatively at least 60 percent, alternatively at least 70 percent, alternatively at least 80 percent, alternatively at least 90 percent, or alternatively 100 percent.

In some embodiments, the presence of the IGF2-based masking domain may reduce the binding affinity of the therapeutic biologic to its antigen by at least about 2-fold, alternatively at least about 5-fold, alternatively at least about 10-fold, alternatively at least about 100-fold, alternatively at least about 1000-fold, alternatively at least about 10000-fold. The removal of the IGF2-based masking domain from the shielded biologic by TavoPRECISE-Shield by protease-mediated cleavage can restore the binding affinity of the converted antibody in antigen binding by at least about 2-fold, alternatively at least about 5-fold, alternatively at least about 10-fold, alternatively at least about 100-fold, alternatively at least about 1000-fold, alternatively at least about 10000-fold.

In some embodiments, the IGF2-based masking domain, when fused to a therapeutic biologic as a shielded biologic by TavoPRECISE-Shield, can inhibit or block the functional activity of the therapeutic biologic to its drug target. When the masking domain is cleaved off by protease, the shielded biologic by TavoPRECISE-Shield is converted to an active biologic with the restoration of the functional activity of the antibody to its drug target.

In vitro and cell-based functional assays are well described in the art for use in determining the functional activity of the shielded biologics by TavoPRECISE-Shield, active biologics and converted biologics after protease cleavage. For example, proinflammatory cytokines including TNFα and IL-1β can bind to and activate their cell surface receptors which leads to the activation of downstream signaling pathways. The effects of antibodies described herein on the neutralization of TNFα and IL-1β activity can be assessed by reporter-based cell assays, cell proliferation assays and cytokine release assays. For cancer drug targets including HER2 and VEGF, the effects of antibodies described herein on the inhibition of HER2 and VEGF mediated signaling transduction can be assessed by reporter-based cell assays and cell proliferation assays. Besides, the effector functions of anti-HER2 antibody including ADCC can be assessed by reporter-based ADCC assay and NK cell-mediated ADCC assay. For immuno-oncology drug targets including PD-L1/PD1 and CTLA-4, the effects of antibodies described herein on PD-L1, PD1, and CTLA-4 mediated T cell inhibition can be assessed by reporter-based cell assays, T cell activation and proliferation assays, and T cell mediated cytotoxicity assays. In addition, the effector functions of anti-PD-L1, PD1 and CTLA-4 antibodies can be assessed by reporter-based ADCC assay and NK cell-mediated ADCC assay.

In some embodiments, the presence of the IGF2-based masking domain may inhibit the functional activities of therapeutic biologics described herein by at least 10 percent, alternatively at least 20 percent, alternatively at least 30 percent, alternatively at least 40 percent, alternatively at least 50 percent, alternatively at least 60 percent, alternatively at least 70 percent, alternatively at least 80 percent, alternatively at least 90 percent, or alternatively 100 percent. The removal of IGF2-based masking domain from the shielded biologics by TavoPRECISE-Shield by protease-mediated cleavage can restore the functional activities of therapeutic biologics described herein by at least 10 percent, alternatively at least 20 percent, alternatively at least 30 percent, alternatively at least 40 percent, alternatively at least 50 percent, alternatively at least 60 percent, alternatively at least 70 percent, alternatively at least 80 percent, alternatively at least 90 percent, or alternatively 100 percent.

Besides in vitro functional activity, animal models and ex vivo functional assays are well described in the art for use in determining the in vivo efficacy of the shielded biologics by TavoPRECISE-Shield, active biologics and converted biologics after protease cleavage. For example, proinflammatory cytokines including TNFα and IL-1β mediate the development of inflammation in disease tissues in various murine inflammation and auto-immune models. The efficacies of anti-TNFα and IL-1β antibodies described herein on the neutralization of TNFα and IL-1β mediated inflammation can be assessed by these animal models. For cancer drug targets including HER2 and VEGF, the anti-tumor efficacy of anti-HER2 and anti-VEGF antibodies described herein can be assessed by tumor xenograft mouse models. For immuno-oncology drug targets including PD-L1/PD1 and CTLA-4, the efficacy of antibodies described herein can be assessed by appropriate murine syngeneic tumor models. In some embodiments, the shielded biologics by TavoPRECISE-Shield with IGF2-based masking domain may show efficacy comparable with the active therapeutic biologics (the therapeutic biologics not in shielded form) in disease models due to the disease site specific conversion of the shielded biologics by TavoPRECISE-Shield into active biologics owing to the removal of the IGF2-based masking domain in the disease site by overexpressed proteases.

Besides efficacy evaluation, animal models and ex vivo functional assays are well described in the art for use in determining the toxicity of the shielded biologics by TavoPRECISE-Shield, active biologics and converted biologics after protease cleavage. For example, blocking the activity of proinflammatory cytokines including TNFα may disrupt host defense on bacterial infection. The toxicity profile of anti-TNFα antibodies described herein on the survival of a mouse with bacterial infection can be assessed by relevant animal models. For cancer drug targets including HER2 and VEGF, blocking these targets may lead to cardiotoxicity. The toxicity profile of anti-HER2 and VEGF antibodies described herein can be assessed by appropriate cardiotoxicity animal models. The shielded biologics by TavoPRECISE-Shield with IGF2-based masking domain may show improved safety profile relative to the active therapeutic biologics (the therapeutic biologics not in shielded form) in disease models due to the systematic masking of the activity of the therapeutic antibodies by the IGF2-based masking domain in the shielded biologics by TavoPRECISE-Shield. In some embodiments, the shielded biologics by TavoPRECISE-Shield with IGF2-based masking domain may reduce the toxicity effects of the corresponding therapeutic biologics by at least about 2-fold, alternatively at least about 5-fold, alternatively at least about 10-fold, alternatively at least about 100-fold, alternatively at least about 1000-fold, alternatively at least about 10000-fold.

### Pharmaceutical Compositions

The shielded biologics by TavoPRECISE-Shield with a masking domain (e.g., IGF2-based) according to the present disclosure can be formulated in compositions, especially pharmaceutical compositions, for use in the methods disclosed herein. Such compositions comprise a therapeutically or prophylactically effective amount of a shielded biologic by TavoPRECISE-Shield with a masking domain (e.g., IGF2-based) described in this disclosure in mixture with a suitable pharmaceutical carrier, e.g., a pharmaceutically acceptable agent. Typically, a shielded biologic by TavoPRECISE-Shield with IGF2-based masking domain described in this disclosure is sufficiently purified for administration to an animal before formulation in a pharmaceutical composition.

Pharmaceutically acceptable agents include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the shielded biologics by TavoPRECISE-Shield (e.g., bolus, depot effect) with sustained release and/or increased stability or half-life in a particular local environment.

### Methods of Use

The shielded biologics by TavoPRECISE-Shield with a masking domain (e.g., IGF2-based) described herein is useful for the treatment of diseases intended for the corresponding therapeutic antibodies, including inflammatory diseases, autoimmune disorders, cancers, neurological diseases, metabolic diseases and organ or tissue transplant rejection. In contrast to corresponding therapeutic antibodies, the shielded biologics by TavoPRECISE-Shield with a masking domain (e.g., IGF2-based) may have comparable efficacy in treating these diseases due to the conversion of the shielded biologics by TavoPRECISE-Shield to active antibodies specifically in disease sites by the removal of the IGF2-based masking domain by proteases overexpressed in disease sites. However, the shielded biologics by TavoPRECISE-Shield with a masking domain (e.g., IGF2-based) described herein may have reduced systemic toxicity due to the masking of the antibody activity by the IGF2-based masking domain in normal tissues without sufficient amount of proteases needed to cleave off the masking domain. In short, the shielded biologics by TavoPRECISE-Shield with a masking domain (e.g., IGF2-based) described herein may be efficacious as the corresponding therapeutic antibody in treating diseases but with much improved safety profile. Due to the improved safety profile, increased amounts of pharmaceutical compositions comprising the shielded biologics by TavoPRECISE-Shield with the IGF2-based masking domain may be administered to the patient with improved treatment efficacy.

In some embodiments, the disclosure provides a method of treating auto-immune/inflammatory diseases comprising administering to a subject a therapeutically effective amount of a shielded biologic by TavoPRECISE-Shield with an IGF2-based masking domain against drug targets including, but are not limited to, TNFα and IL-1β. The disclosure provides use of the shielded biologics by TavoPRECISE-Shield with the IGF2-based masking domain provided herein in a method of treating the auto-immune/inflammatory diseases; and for use of the shielded biologics by TavoPRECISE-Shield provided herein in the manufacture of a medicament for use in the auto-immune/inflammatory diseases. Exemplary auto-immune and/or inflammatory diseases include, but are not limited to, the following: rheumatoid arthritis, systemic lupus erythematosus, osteoarthritis, ankylosing spondylitis, Behcet's Disease, gout, psoriatic arthritis, multiple sclerosis, Crohn's colitis, and inflammatory bowel disease.

In some embodiments, the disclosure also provides a method of treating diabetes, nerve, eye, skin diseases comprising administering to a subject a therapeutically effective amount of a shielded biologic by TavoPRECISE-Shield with an IGF2-based masking domain against drug targets, including but are not limited to, TNFα and IL-1β. The disclosure also provides use of the shielded biologics by TavoPRECISE-Shield with the IGF2-based masking domain provided herein in a method of treating such diabetes, nerve, eye, and skin diseases; and for use of the shielded biologics by TavoPRECISE-Shield provided herein in the manufacture of a medicament for use in such diabetes, nerve, eye, and skin diseases. Exemplary diseases include but are not limited to: Type II diabetes mellitus, Parkinson's disease, age-related macular degeneration, polyneuropathy, sensory peripheral neuropathy, proliferative diabetic retinopathy, diabetic neuropathy, decubitus ulcer, fulminant Type 1 diabetes, retinal vasculitis, non-infectious posterior uveitis, alcoholic neuropathy.

In some embodiments, the disclosure also provides a method of treating cancer comprising administering to a subject a therapeutically effective amount of a shielded biologic by TavoPRECISE-Shield with an IGF2-based masking domain against drug targets including, but are not limited to, HER2, VEGF, EGFR, PD-L1, PD1 and CTLA-4, CD47, CD24. The disclosure also provides use of the shielded biologics by TavoPRECISE-Shield with the IGF2-based masking domain provided herein in a method of treating cancer; and for use of the shielded biologics by TavoPRECISE-Shield provided herein in the manufacture of a medicament for use in cancer. Exemplary cancers include, but are not limited to: multiple myeloma, non-small cell lung cancer, acute myeloid leukemia, female breast cancer, pancreatic cancer, colorectal cancer and peritoneum cancer.

In some embodiments, the disclosure also provides a method of treating chronic viral infection comprising administering to a subject a therapeutically effective amount of a shielded biologic by TavoPRECISE-Shield with an IGF2-based masking domain against drug targets including, but are not limited to, PD-L1, PD1 and CTLA-4. The disclosure also provides use of the shielded biologics by TavoPRECISE-Shield with the IGF2-based masking domain provided herein in a method of treating chronic viral infection; and for use of the shielded biologics by TavoPRECISE-Shield provided herein in the manufacture of a medicament for use in chronic viral infection. Exemplary chronic viral infection includes, but are not limited to: HPV, HBV, EBV, HSV.

All combinations of the various elements described herein are within the scope of the disclosure unless otherwise indicated herein or otherwise clearly contradicted by context.

This disclosure will be better understood from the Experimental Details, which follow. However, one skilled in the art will readily appreciate that the specific methods and results discussed are merely illustrative of the disclosure.

### EXAMPLES

The following examples are provided to describe the disclosure in greater detail. They are intended to illustrate, not to limit, the disclosure.

### Example 1: Expression and Purification of Anti-TNFα Shielded Antibody with IGF2-Based Masking Domain

An anti-TNFα antibody was employed to evaluate the feasibility of masking the antibody activity with IGF2-based masking domain in the following studies. Heavy chain and light chain constructs expressing the anti-TNFα shielded antibodies listed in Table 3 were designed. These shielded biologics by TavoPRECISE-Shield all have an IGF2-based masking domain fused to the N-terminus of the TNFα antibody heavy chains based on ADA-H2X variable domain and TNFα light chains based on ADA-L1 variable domain while the protease cleavable linker sequences are different among them. Five of them have different substrate sequences for MMP2/MMP9 and two of them have substrate sequences for MMP3 and uPA respectively (Table 3). As a control, the heavy chain construct (EAC167) and light chain construct (EAC127) of the anti-TNFα antibody without the IGF2-based masking domain (TAVO167127) were also designed.

TABLE 3
Anti-TNFα shielded antibodies with
IGF2-based masking domain

| | | | | Protease |
|---|---|---|---|---|
| Shielded | Heavy | Light | | substrate |
| antibody | chain | chain | protease | sequence |
| TAVO186187 | EAC186 | EAC187 | MMP2/9 | GPLGVR |
| | | | | (SEQ ID |
| | | | | NO: 33) |
| TAVO188189 | EAC188 | EAC189 | MMP2/9 | PLGLAR |
| | | | | (SEQ ID |
| | | | | NO: 34) |
| TAVO190191 | EAC190 | EAC191 | MMP2/9 | PLGLAG |
| | | | | (SEQ ID |
| | | | | NO: 35) |
| TAVO192193 | EAC192 | EAC193 | MMP2/9 | IPVSLRSG |
| | | | | (SEQ ID |
| | | | | NO: 36) |
| TAVO194195 | EAC194 | EAC195 | MMP2/9 | GPLGMLSQ |
| | | | | (SEQ ID |
| | | | | NO: 37) |
| TAVO196197 | EAC196 | EAC197 | MMP3 | RPKPVEVWRK |
| | | | | (SEQ ID |
| | | | | NO: 38) |
| TAVO198199 | EAC198 | EAC199 | uPA | LSGRSDNH |
| | | | | (SEQ ID |
| | | | | NO: 39) |

Plasmids encoding heavy chains and light chains of these anti-TNFα shielded antibody with IGF2-based masking domain were co-transfected into Expi293F cells following the transfection kit instructions (Thermo Scientific). Cells were spun down five days post transfection, and the supernatant were passed through a 0.2 μm filter. The purification of the expressed shielded biologics by TavoPRECISE-Shield in supernatant was carried out by affinity chromatography over protein A agarose column (GE Healthcare Life Sciences). The purified shielded biologics by TavoPRECISE-Shield were buffer-exchanged into DPBS, pH7.2 by dialysis, and protein concentrations were determined by UV absorbance at 280 nm.

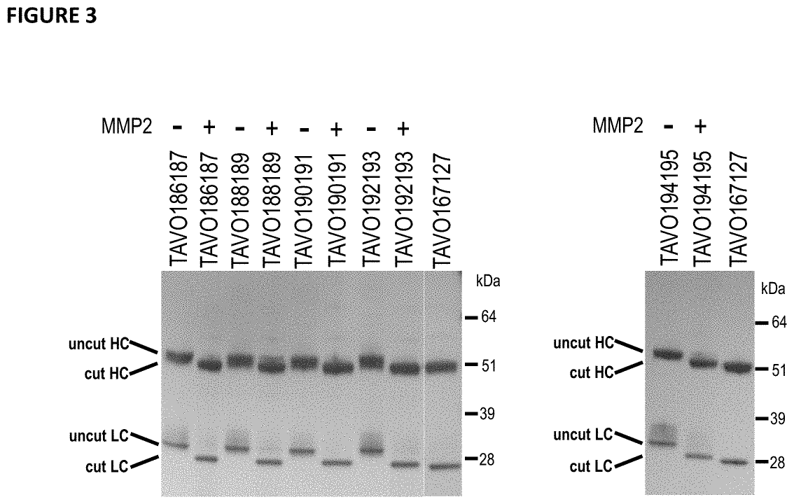

The purified anti-TNFα shielded antibody were subjected to SDS-PAGE analysis (FIG. 3, 4). Under the reduced condition, all the shielded antibodies had heavy chains and light chains with the molecular weights slightly bigger than the ones of TAVO167127, owing to the fusion of the IGF2 A chain and B chain to the native antibody heavy chain and light chain respectively.

### Example 2: Digestion of Shielded Antibody by TavoPRECISE-Shield with IGF2-Based Masking Domain by Proteases

In vitro protease cutting assays were set up to evaluate whether the IGF2-based masking domain can be removed from the anti-TNFα shielded antibody by proteases.

For MMP2, recombinant human MMP2 was activated by incubating with p-amino phenylmercuric acetate (APMA) according to manufacturer's instruction (R&D Systems). 10 μg of TAVO186187, TAVO188189, TAVO190191, TAVO192193, and TAVO194195 were incubated with 50 ng of activated MMP2 overnight at 37° C. The digestions of the five anti-TNFα shielded antibodies were evaluated by SDS-PAGE under reduced condition. It was observed that the molecular weights of the heavy chain and light chain for the digested anti-TNFα shielded antibodies were smaller relative to the corresponding undigested shielded antibodies, with size comparable to that for TAVO167127, which is the anti-TNFα antibody without attaching the IGF2-based masking domain (FIG. 3). This indicated the efficient cleavage of the different MMP2-cleavable linker sequences of the five anti-TNFα shielded antibodies by MMP2.

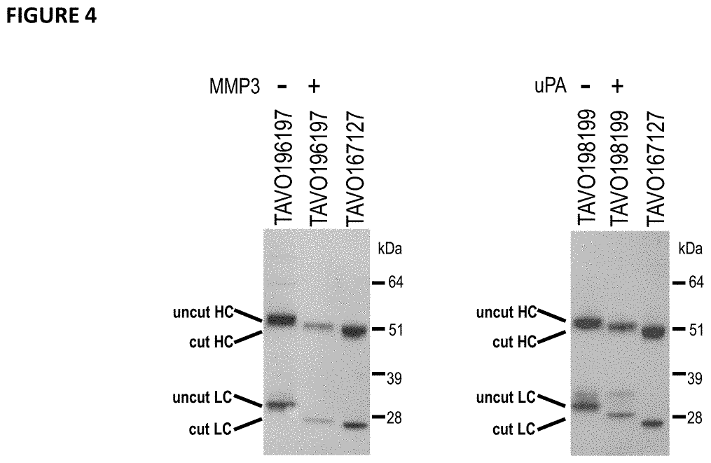

For MMP3, recombinant human MMP3 was activated by incubating with Chymotrypsin for 30 minutes according to manufacturer's instruction (R&D Systems). 10 μg of the anti-TNFαshielded antibodies TAVO196197 was incubated with 50 ng of activated MMP3 overnight at 37° C. The digestions of TAVO196197 were evaluated by SDS-PAGE under reduced condition. It was observed that the molecular weights of the heavy chain and light chain for the digested TAVO196197 became smaller after digestion relative to the corresponding undigested shielded antibody, with size comparable to that for TAVO167127 (FIG. 4). This indicated the efficient cleavage of TAVO196197 with MMP3-cleavable linker sequence by MMP3.

uPA is a protease that does not need pre-activation. 10 μg of the anti-TNFα shielded antibody TAVO198199 was incubated with 500 ng of recombinant human uPA (R&D Systems) overnight at 37° C. The digestions of TAVO198199 were evaluated by SDS-PAGE under reduced condition. It was observed that the molecular weights of heavy chain and light chain for the digested TAVO198199 became smaller after digestion relative to the corresponding undigested shielded antibody and with size comparable to that for TAVO167127 (FIG. 4). This indicated the efficient cleavage of TAVO198199 with uPA-cleavable linker sequence by uPA.

### Example 3: Binding Affinity to TNFα by Anti-TNFα Shielded Antibody Before and After Protease Digestion

An ELISA-based binding assay was employed to evaluate the binding to TNFα by anti-TNFα shielded antibody with IGF2-based masking domain before and after protease cleavage. In this assay, 1 μg/mL recombinant human TNFα (R&D systems) was coated on an ELISA plate. Increasing concentrations of anti-TNFα shielded antibodies were applied on the plate and their binding to the recombinant human TNFα was detected by HRP-conjugated anti-human secondary antibody.

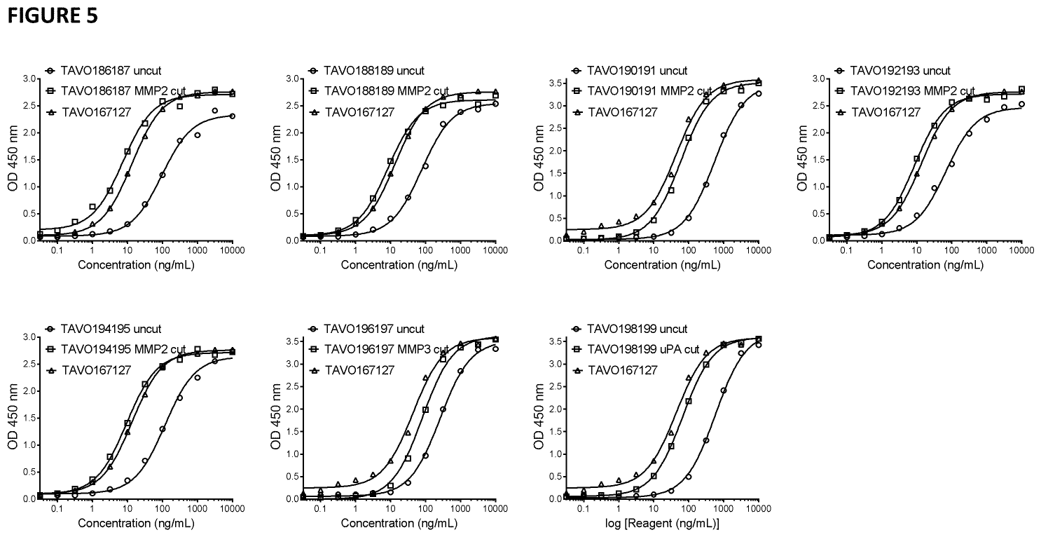

It was observed that the anti-TNFα shielded antibodies with IGF2-based masking domain dose-dependently bound human TNFα, however, their binding affinity were at least 5-fold lower than that of TAVO167127, which is the active anti-TNFα antibody without the IGF2-based masking domain (FIG. 5). Upon removal of the IGF2-based masking domain from these anti-TNFα shielded antibodies by protease cleavage, the digested shielded antibodies showed binding affinity comparable to that of TAVO167127, indicating the conversion to active anti-TNFα antibodies from shielded antibodies.

### Example 4: Neutralization of TNFα Activity by Anti-TNFα Shielded Antibody Before and After Protease Digestion

A HEK-Blue TNFα reporter assay was developed to assess functional activity of TNFα. In this assay, HEK-Blue TNFα cells (Invivogen) expressing TNFα receptor and a secreted embryonic alkaline phosphatase (SEAP) reporter gene was employed. Human TNFα could dose-dependently induce reporter gene expression. The HEK-Blue TNFα reporter assay was then employed to evaluate the neutralization of TNFα activity by the anti-TNFα shielded antibody with IGF2-based masking domain before and after protease cleavage. Increasing amounts of the shielded antibodies, along with 3 ng/mL human TNFα, were applied to HEK-Blue TNFα reporter cells. After overnight incubation, the SEAP reporter gene expression was quantitated.

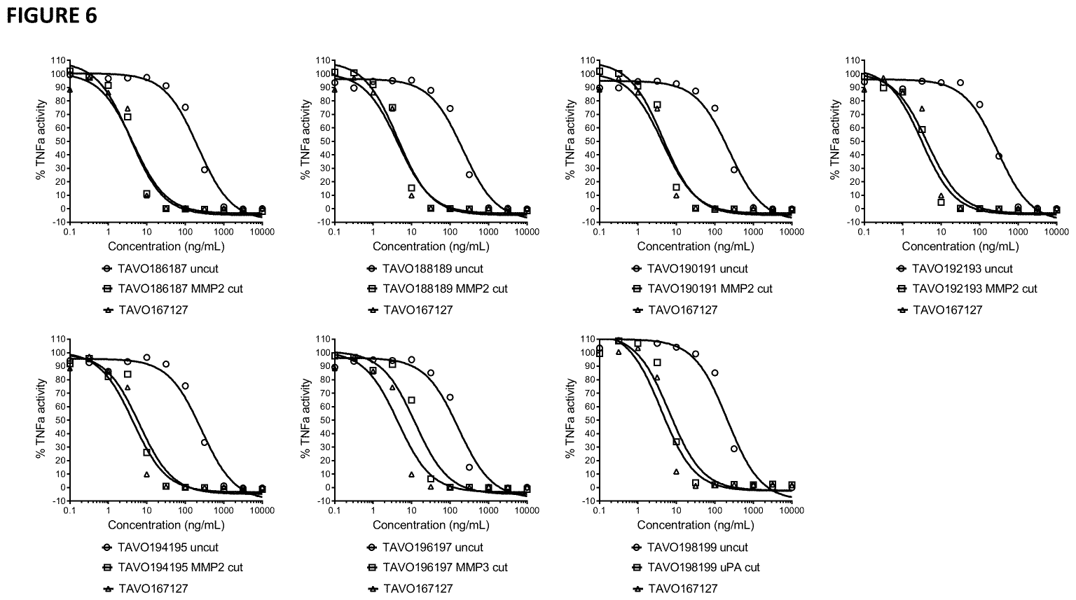

In this example, the anti-TNFα shielded antibodies with IGF2-based masking domain neutralized the reporter gene activation by human TNFα in a dose-dependent manner. However, their potency in blocking TNFα functional activity was about 50-fold lower than that of TAVO167127, which was the active anti-TNFα antibody without the IGF2-based masking domain (FIG. 6). Upon removal of the IGF2-based masking domain from these anti-TNFα shielded antibodies by protease cleavage, the digested antibodies showed functional potencies comparable to that of TAVO167127, indicating the enzymatic conversion from shielded antibodies to active anti-TNFα antibodies.

### Example 5: Characterization of Anti-TNFα Shielded Antibody with Two Different Types of Protease-Cleavable Linkers

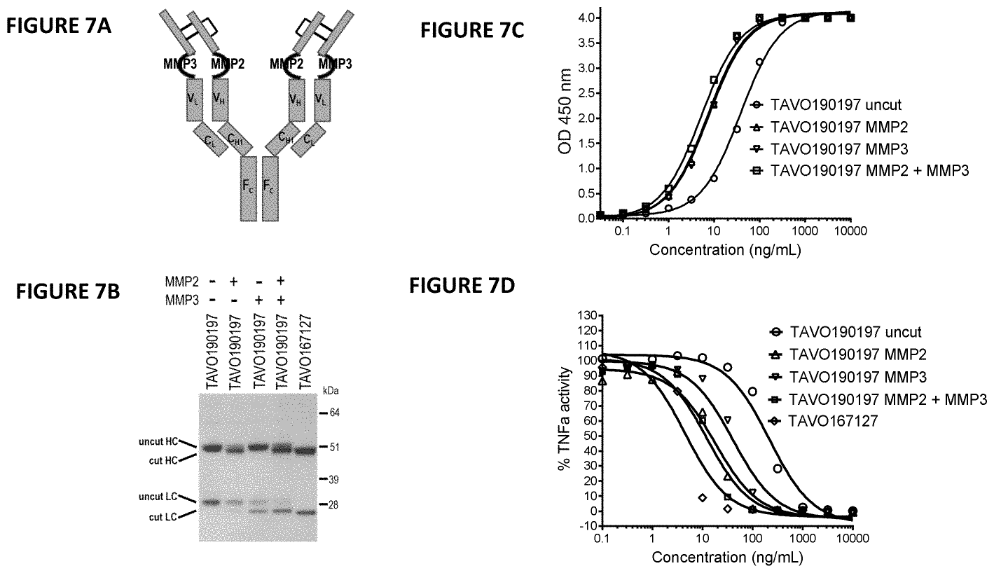

In this study, an anti-TNFα shielded antibody TAVO190197 (by pairing heavy chain EAC190 with light chain EAC197 (Table 2)) was generated in which the protease-cleavable linker sequence between the IGF2 A chain and the antibody heavy chain comprises a substrate sequence cleavable by MMP2 while the protease-cleavable linker sequence between the IGF2 B chain and the antibody light chain comprises a substrate sequence cleavable by MMP3 (FIG. 7A). The susceptibility of TAVO190197 to digestion by MMP2, MMP3 or combination of MMP2 and MMP3 was evaluated by reduced SDS-PAGE. As shown, the heavy chain of TAVO190197 was susceptible to MMP2 digestion while the light chain was not susceptible to MMP2 digestion due to the presence of MMP2-cleavable linker on the heavy chain but not on the light chain. On the other hand, the light chain of TAVO190197 was susceptible to MMP3 digestion while the heavy chain was not susceptible to MMP3 digestion due to the presence of MMP3 cleavable linker on the light chain but not on the heavy chain. The combination of both MMP2 and MMP3 can cleave apart both the heavy chain and light chain of TAVO190197 (FIG. 7B).

An ELISA-based binding assay was employed to evaluate the binding to TNFα by TAVO190197 before and after protease cleavage. It was observed that TAVO190197 after digestion by MMP2 alone or MMP3 alone dose-dependently bound human TNFα with greater binding affinity to TNFα than undigested TAVO190197. The binding affinities of TAVO190197 after digestion by MMP2 alone or MMP3 alone were comparable to that digested by both MMP2 and MMP3 (FIG. 7C). HEK-Blue TNFαreporter assay was employed to evaluate the neutralization of TNFα activity by TAVO190197 before and after protease cleavage. It was observed that TAVO190197 after digestion by MMP2 alone or MMP3 alone dose-dependently neutralized TNFα activity with much greater efficacy than undigested TAVO190197. Although the functional activity of TAVO190197 after digestion by MMP3 was weaker, likely due to non-complete cut, the functional activity of TAVO190197 after digestion by MMP2 was comparable to that digested by both MMP2 and MMP3 (FIG. 7D).

This data indicated that for the shielded antibody with the heavy chain and light chain of the antibody linked to the IGF2 chains via two different types of protease-cleavable linkers, either type of the proteases may cleave apart one of the two linkers and make the masking domain “cleaved open” and the shielded antibody may be converted into an active antibody. This particular design of shielded biologics by TavoPRECISE-Shield allows the shielded biologics by TavoPRECISE-Shield to be activated in a disease tissue in which at least one of the two types of proteases is present.

### Example 6: Characterization of Variant IGF2 Masking Domain with Minimal Binding to IGF2 Receptor

The utility of native IGF2 sequences as the masking domain may raise the concern of toxic effects due to IGF2 signaling, including aberrant modulation of glucose metabolism and cell proliferation. To ameliorate this concern, variant IGF2-based masking domains comprising the A chain and B chain of human IGF2 with mutations that disrupt IGF2 binding to its receptors were thus designed and evaluated. More specifically, a heavy chain polypeptide designated as EAC271 with sequence set forth as SEQ ID NO. 73 was designed with identical sequence as EAC190, except that its A chain of IGF2 contains a V43L mutation that disrupts IGF2 binding to its receptor. Besides, a light chain polypeptide designated as EAC272 with sequence set forth as SEQ ID NO. 74 was designed with identical sequence as EAC191, except that its B chain of IGF2 contains a Y27A mutation that disrupts IGF2 binding to its receptor. By combination pairings of the heavy chains of EAC271 and EAC190 with the light chains of EAC272 and EAC191, four anti-TNFα shielded antibodies, TAVO190191, TAVO271191, TAVO190272 and TAVO271272, were generated. TAVO190191 has native IGF2 chain sequences, TAVO271191 has the A chain mutation V43L, TAVO190272 has the B chain mutation Y27A, and TAVO271272 has the V43L and Y27A double mutations on its IGF2 chains.

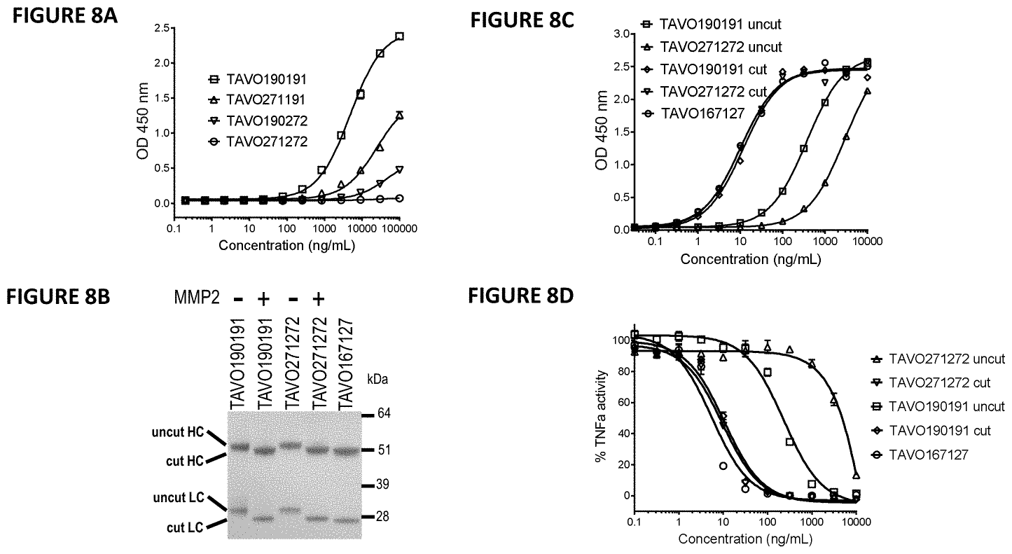

The bindings of these four anti-TNFα shielded antibodies to IGF2 receptor were evaluated by ELISA assay. 1 μg/mL of IGF2 receptor (IGF2R, R&D Systems) was coated on ELISA plate and an increasing amount of anti-TNFα shielded antibodies were applied to the plate to evaluate their binding to IGF2R. While TAVO190191 showed dose-dependent binding to IGF2R, both TAVO271191 and TAVO190272 showed reduced binding, and TAVO271272 showed no binding to IGF2R (FIG. 8A). The susceptibility of TAVO271272 to digestion by MMP2 was evaluated by SDS-PAGE. It was observed that MMP2 could efficiently cleaved off the variant IGF2 masking domain from TAVO271272 as that from TAVO190191 (FIG. 8B). This indicated that the V43L and Y27A mutations in IGF2 chains did not affect the cutting by MMP2.

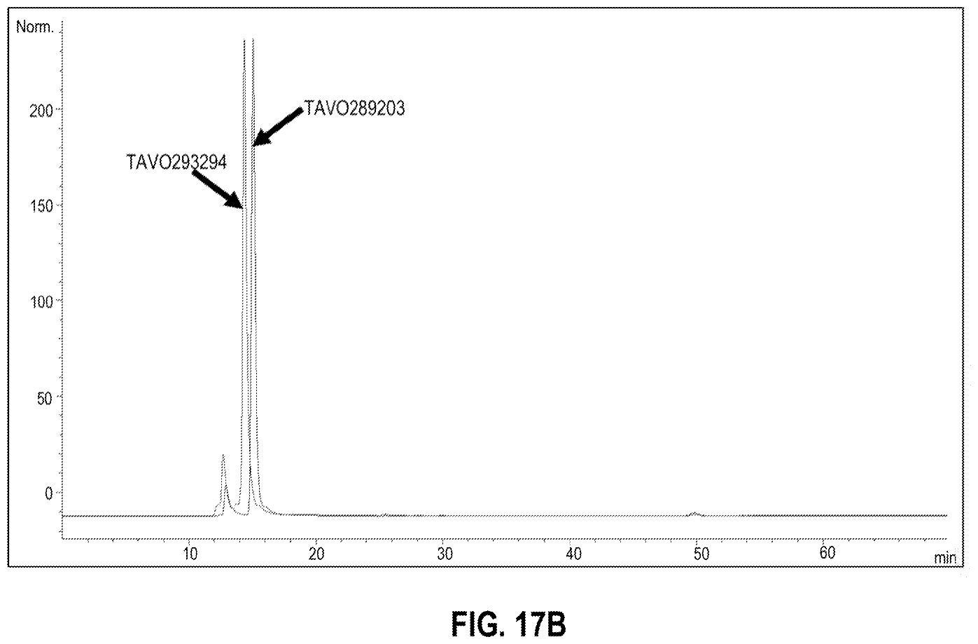

Besides SDS-PAGE, TAVO271272 with variant IGF2 masking domain and TAVO167127 without the masking domain were subjected to size exclusion chromatography. Both antibodies migrated as a major monomeric protein peak (FIG. 17A). Judging from the timing of protein peak appearance, TAVO271272 has a slightly larger molecular weight due to the presence of the IGF2 masking domain.

An ELISA-based binding assay was employed to evaluate the binding to TNFα by TAVO271272 and TAVO190191 before and after MMP2 protease cleavage. It was observed that TAVO271272 with variant IGF2 masking domain had 8-folds more reduced binding affinity to human TNFα than TAVO190191 with native IGF2 masking domain (FIG. 8C). This indicated a better masking efficiency for IGF2 masking domain with V43L/Y27A mutations. Upon removal of the IGF2-based masking domain from these anti-TNFα shielded antibodies by MMP2 protease cleavage, both TAVO271272 and TAVO190191 showed binding affinities comparable to that of TAVO167127, indicating the conversion to the active anti-TNFαantibodies from the shielded antibodies.

An HEK-Blue reporter assay was employed to evaluate the functional neutralization of TNFα by TAVO271272 and TAVO190191 before and after MMP2 protease cleavage. It was observed that TAVO271272 with variant IGF2 masking domain had even more reduced potency in neutralization to human TNFα than TAVO190191 with native IGF2 masking domain (FIG. 8D). This indicated a better masking efficiency for IGF2 masking domain with V43L/Y27A mutations. Upon removal of the IGF2-based masking domain from these anti-TNFα shielded antibodies by MMP2 protease cleavage, both TAVO271272 and TAVO190191 showed neutralization activities to TNFα comparable to that of TAVO167127, indicating the conversion to the active anti-TNFα antibodies from the shielded antibodies.

The variant IGF2 masking domain was also engineered on additional anti-TNFα antibodies. More specifically, anti-TNFα antibody heavy chain constructs designated as EAC578, EAC579, EAC580, EAC584 with sequences set forth as SEQ ID NO. 75, 76, 77, 78 respectively were designed with IGF2 A chain containing V43L mutation fused to anti-TNFα antibody heavy chains based on ADA-H1, ADA-H1X, ADA-H2 and ADA-H4X variable domains respectively. Besides, an anti-TNFαantibody light chain construct designated as EAC585 with sequence set forth as SEQ ID NO. 79 was designed with IGF2 B chain containing Y27A mutation fused to anti-TNFαantibody light chain based on ADA-L2 variable domain. By combination pairings of heavy chains of EAC271, EAC578, EAC579, EAC580, EAC584 with light chains of EAC272 and EAC585, anti-TNFαshielded antibodies with the variant IGF2 masking domain listed in Table 4 were designed.

TABLE 4
Anti-TNFα shielded antibodies
with variant IGF2-based masking domain

| Shielded | | | | |
|---|---|---|---|---|
| antibody | Heavy chain | Light chain | V_{H} | V_{L} |
| TAVO271272 | EAC271 | EAC272 | ADA-H2X | ADA-L1 |
| TAVO578272 | EAC578 | EAC272 | ADA-H1 | ADA-L1 |
| TAVO579272 | EAC579 | EAC272 | ADA-H1X | ADA-L1 |
| TAVO580272 | EAC580 | EAC272 | ADA-H2 | ADA-L1 |
| TAVO584272 | EAC584 | EAC272 | ADA-H4X | ADA-L1 |
| TAVO271585 | EAC271 | EAC585 | ADA-H2X | ADA-L2 |
| TAVO578585 | EAC578 | EAC585 | ADA-H1 | ADA-L2 |
| TAVO579585 | EAC579 | EAC585 | ADA-H1X | ADA-L2 |
| TAVO580585 | EAC580 | EAC585 | ADA-H2 | ADA-L2 |
| TAVO584585 | EAC584 | EAC585 | ADA-H4X | ADA-L2 |

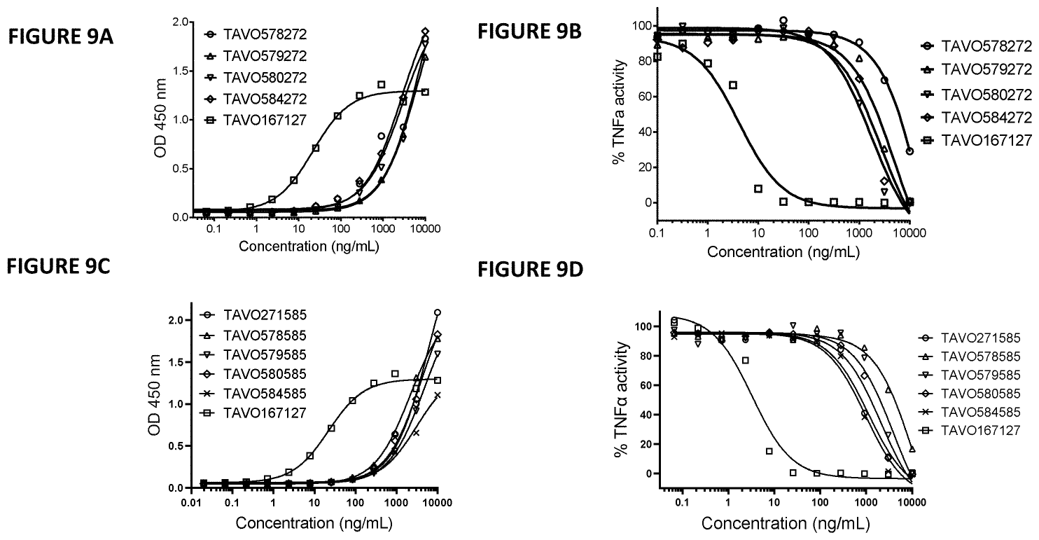

Anti-TNFα shielded antibodies with the variant IGF2 masking domain listed in Table 4 were generated by transient transfection into Expi293F cells and purification over protein A column. Many of them showed better yields than TAVO271272 under the same condition. The variant IGF2 masking domains from all of them could be efficiently removed by cutting with MMP2 protease. By ELISA-based binding assays, it was observed that all of these anti-TNFα shielded antibodies with the variant IGF2 masking domain had reduced binding affinities to human TNFα relative to unmasked anti-TNFα antibody TAVO167127 due to masking (FIG. 9A, 9C). In addition, by HEK-Blue TNFα reporter assay, all of these anti-TNFα shielded antibodies with the variant IGF2 masking domain had reduced potency in neutralization of TNFα relative to unmasked anti-TNFαantibody TAVO167127 due to masking (FIG. 9B, 9D).

### Example 7: In Vivo Efficacy of Anti-TNFα Shielded Antibody with IGF2-Based Masking Domain in a Model of Collagen Antibody Induced Arthritis

The efficacy of the anti-TNFα shielded antibody with IGF2-based masking domain in inflammation was evaluated in a collagen antibody induced arthritis (CAIA) model (Moore, Allden et al. 2014). CAIA model was established through the administration of an anti-collagen monoclonal antibody cocktail and the subsequent administration of lipopolysaccharide (LPS). CAIA is characterized by inflammation, pannus formation and bone erosions similar to those observed in RA. Since anti-human TNFα antibody cannot neutralize mouse TNFα activity even though there was good binding affinity to mouse TNFα, the study was conducted using Tg1278/TNFKO mice which lacks murine TNFα and are heterozygous for multiple copies of the human TNFα transgene that is expressed under normal physiological control.

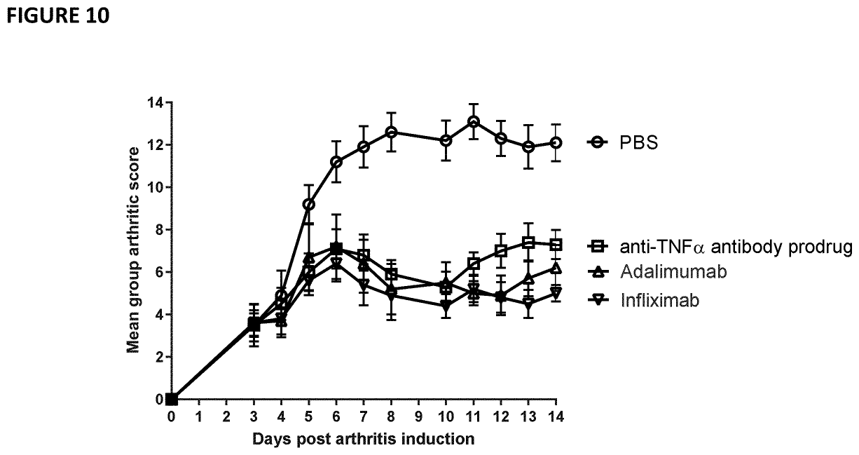

CAIA was induced in 8 to 10-week-old Tg1278/TNFKO male mice that received intraperitoneal injections (i.p.) of arthritogenic antibody cocktail (ArthritoMab, MD Biosciences) on day 0, followed by an i.p. injection of LPS on Day 3. After CAIA induction, PBS, anti-TNFα shielded antibody, or reference anti-TNFα antibodies Adalimumab and Infliximab were dosed at 5 mg/kg twice per week for two weeks. The clinical scores of arthritis, histopathology of the limbs and body weight were measured and collected as the read out. The in vivo efficacy of anti-TNFα shielded antibody with IGF2-based masking domain was compared to that for reference anti-TNFα antibodies without the masking domain. It was observed that anti-TNFα shielded antibody had comparable efficacy in blocking CAIA-induced limb inflammation to Adalimumab and Infliximab (FIG. 10).

### Example 8: In Vivo Efficacy of Anti-TNFα Shielded Antibody with IGF2-Based Masking Domain in a Model of Knee Joint Inflammation

A mouse model of knee joint inflammation will also be developed to evaluate the in vivo efficacy of anti-TNFα shielded antibody in normal mice. The joint inflammation in this model is induced upon continuous secretion of human TNFα from transfected mouse NIH3T3 cells injected into one of the knee joints, since human TNFα can activate cognate receptors in mice to induce inflammation. This model allows the study of anti-TNFα antibodies which can neutralize the effects of human cytokines but lack the cross-reactivity to murine cytokines.

For the development of this model, murine fibroblast cell line NIH3T3, derived from a DBA-1 mouse background, will be transfected with constructs expressing human TNFα and NIH3T3 cell lines stably expressing TNFα is thus established. To study the in vivo efficacy of anti-TNFα shielded antibody, it will be dosed intraperitoneally into the DBA-1 mice two hours prior the mice is given an intra-articular (IA) injection of 10×10⁴ NIH3T3: human TNFα cells into the right knee joint and 10×10⁴ NIH3T3 parental cells into the left knee as a control. Caliper measurements on both knees will be taken on Day −1, and Days 1, 2 and 3 post injection and knee joint inflammation will be quantitated as the caliper measurement difference between the treated right knee and untreated left knee. The in vivo efficacy of anti-TNFα shielded antibodies with IGF2-based masking domain will be compared to those anti-TNFα antibodies without the masking domain.

### Example 9: In Vivo Toxicity of Anti-TNFα Shielded Antibody with IGF2-Based Masking Domain in a Listeria Infection Model

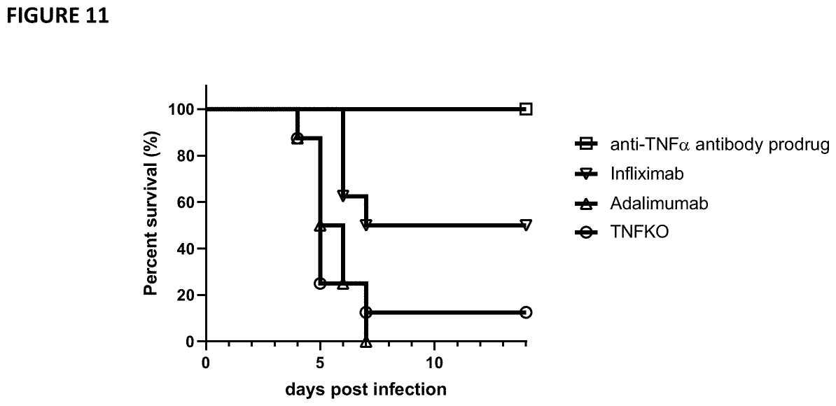

Long-term administration of anti-TNFα antibody may increase patients' susceptibility to serious infection. To investigate whether anti-TNFα shielded antibody with IGF2-based masking domain may have reduced risk of opportunistic infection, a listeria infection model was established. In this model, Tg1278/TNFKO mice, which express normally regulated human TNFα but lack murine TNFα, was intraperitoneally injected with 0.3 mg/kg anti-TNFα shielded antibody with IGF2-based masking domain (shielded biologics by TavoPRECISE-Shield), 0.3 mg/kg anti-TNFα antibody Adalimumab, or 0.3 mg/kg anti-TNFα antibody Infliximab before being challenged with a lethal dose of L. monocytogenes (n=8). TNFKO mice, which lack murine TNFα were used as the positive control. The lethality of antibody treated mice over time was recorded. While Tg1278/TNFKO mice dosed with either Adalimumab or Infliximab showed poor survival rate over the period of 14 days just like the TNFα knockout mice, the eight mice dosed with the anti-TNFα shielded antibody showed 100% survival (FIG. 11). This data revealed the protective effect of the anti-TNFα shielded antibody in which the IGF2-based masking domain was able to shield the anti-TNFα antibody activity.

### Example 10: Expression and Purification Anti-HER2 Shielded Antibody with IGF2-Based Masking Domain

An anti-HER2 antibody Trastuzumab was also employed to evaluate the masking the antibody activity with IGF2-based masking domain. Heavy chain and light chain constructs expressing anti-HER2 shielded antibodies listed in Table 5 were designed. These shielded antibodies all have the IGF2-based masking domain fused to the N-terminus of trastuzumab antibody heavy chain and light chain with MMP2/9 or uPA cleavable linker sequences and IgG1 Fc with different sets of mutations. As controls, two anti-HER2 trastuzumab antibodies without IGF2-based masking domain but with different IgG1 Fc, TAVO202203 and TAVO289203, were also designed. TAVO202203, composed of heavy chain EAC202 and light chain EAC203, has IgG1 with E233P, F405L, M428L, N434S mutations and G236 deleted. TAVO289203, composed of heavy chain EAC289 and light chain EAC203, has IgG1 with F405L, M428L, N434S mutations.

TABLE 5
Anti-HER2 shielded antibodies with
IGF2-based masking domain

| | | | | Protease | |
|---|---|---|---|---|---|
| Shielded | Heavy | Light | pro- | substrate | Fc |
| antibody | chain | chain | tease | sequence | mutation |
| TAVO | EAC224 | EAC225 | MMP2/9 | PLGLAG | IgG1 |
| 224225 | | | | (SEQ ID | with |
| | | | | NO: 35) | E233P, |
| | | | | | F405L, |
| | | | | | M428L, |
| | | | | | N434S |
| | | | | | mutations |
| | | | | | and |
| | | | | | G236 |
| | | | | | deleted |
| TAVO | EAC243 | EAC244 | uPA | LSGRSDNH | IgG1 |
| 243244 | | | | (SEQ ID | with |
| | | | | NO: 39) | E233P, |
| | | | | | F405L, |
| | | | | | M428L, |
| | | | | | N434S |
| | | | | | mutations |
| | | | | | and |
| | | | | | G236 |
| | | | | | deleted |
| TAVO | EAC293 | EAC294 | MMP2/9 | PLGLAG | IgG1 |
| 293294 | | | | (SEQ ID | with |
| | | | | NO: 35) | F405L, |
| | | | | | M428L, |
| | | | | | N434S |
| | | | | | mutations |

Plasmids encoding heavy chains and light chains of these anti-HER2 shielded antibodies with IGF2-based masking domain were co-transfected into Expi293F cells following the transfection kit instructions (Thermo Scientific). Cells were spun down five days post transfection, and the supernatants were passed through a 0.2 μm filter. The purification of the expressed shielded antibodies in supernatant were carried out by affinity chromatography over protein A agarose column (GE Healthcare Life Sciences). The purified shielded antibodies were buffer-exchanged into DPBS, pH7.2 by dialysis, and protein concentrations were determined by UV absorbance at 280 nm.

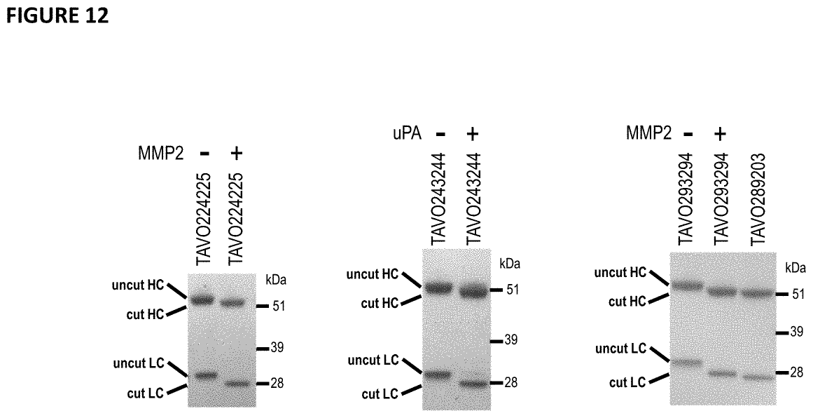

The purified anti-HER2 shielded antibodies were subjected to SDS-PAGE analysis (FIG. 12). Under the reduced condition, the heavy chain and light chain of the anti-HER2 shielded antibody TAVO293294 showed molecular weights slightly bigger than the ones of TAVO289203, owing to the fusion of the IGF2 A chain and B chain to the native antibody heavy chain and light chain, respectively.

Besides SDS-PAGE, TAVO293294 with variant IGF2 masking domain and TAVO289203 without the masking domain were subjected to size exclusion chromatography. Both antibodies migrated as major monomeric protein peaks with a minor aggregated fraction (FIG. 17B). Judging from the retention time of the major protein peak, TAVO293294 has a slightly larger molecular weight due to the presence of the IGF2 masking domain.

### Example 11: Digestion of Anti-HER2 Shielded Antibody with IGF2-Based Masking Domain with Protease

In vitro protease cutting assays were set up to evaluate whether the IGF2-based masking domain can be removed from the anti-HER2 shielded antibodies by proteases. For MMP2, recombinant human MMP2 was activated by incubating with p-amino phenylmercuric acetate (APMA) according to manufacturer's instruction (R&D Systems). 10 μg of TAVO224225 and TAVO293294 were incubated with 50 ng of activated MMP2 overnight at 37° C. The digestions of TAVO224225 or TAVO293294 were evaluated by SDS-PAGE under reduced condition (FIG. 12). The molecular weight of the heavy chain and light chain of digested TAVO224225 or TAVO293294 became smaller relative to the corresponding undigested shielded antibodies and with size comparable to anti-HER2 antibody without the IGF2-based masking domain. uPA is a protease that does not need pre-activation. 10 μg of the anti-HER2 shielded antibody TAVO243244 was incubated with 500 ng of recombinant human uPA (R&D Systems) overnight at 37° C. The digestions of TAVO243244 was evaluated by SDS-PAGE under reduced condition (FIG. 12). The molecular weight of the heavy chain and light chain of digested TAVO243244 became smaller relative to the corresponding undigested shielded biologic by TavoPRECISE-Shield.

### Example 12: Binding Affinity to HER2 by Anti-HER2 Shielded Antibody Before and After Protease Digestion

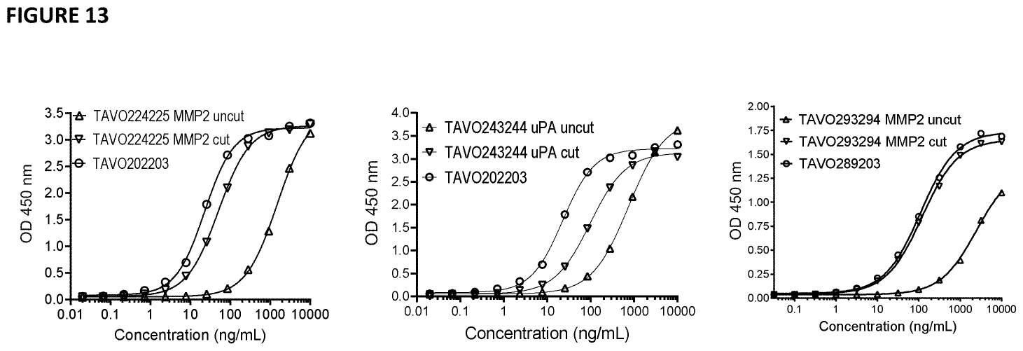

ELISA-based binding assays were employed to evaluate the binding to HER2 by anti-HER2 shielded antibodies with IGF2-based masking domain before and after protease cleavage. In this assay, 1 μg/mL recombinant human HER2 (R&D systems) was coated on an ELISA plate. Increasing concentrations of the anti-HER2 shielded antibodies were applied on the plate and their binding to the recombinant human HER2 were detected by HRP-conjugated anti-human IgG secondary antibody. The anti-HER2 shielded antibodies with IGF2-based masking domain, including TAVO224225, TAVO243244 and TAVO293294, showed much lower binding affinity to HER2 compared to the active anti-HER2 antibodies TAVO202203 or TAVO289203, and the removal of IGF2-based masking domain by protease cleavage largely restored the HER2 binding affinity by the cleaved shielded antibodies (FIG. 13).

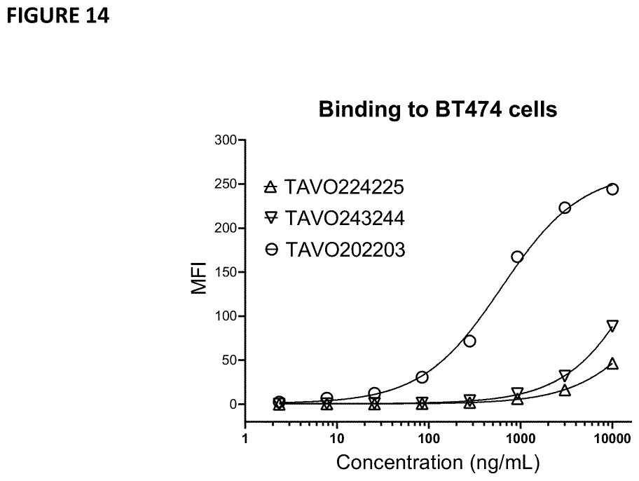

Besides the ELISA-based binding assay with recombinant HER2, a flow cytometry-based binding assay using HER2-expressing breast cancer cell line BT474 was set up to evaluate the binding to cell surface HER2 by anti-HER2 shielded antibodies with IGF2-based masking domain before and after protease cleavage. The shielded anti-HER2 antibody TAVO224225 showed much lower binding affinity to cell surface HER2 compared to the active anti-HER2 antibody TAVO202203, and the removal of IGF2-based masking domain by protease cleavage largely restored the HER2 binding affinity by the cleaved shielded antibody (FIG. 14).

### Example 13: Inhibition of HER2⁺ Cancer Cell Proliferation by Anti-HER2 Shielded Antibody

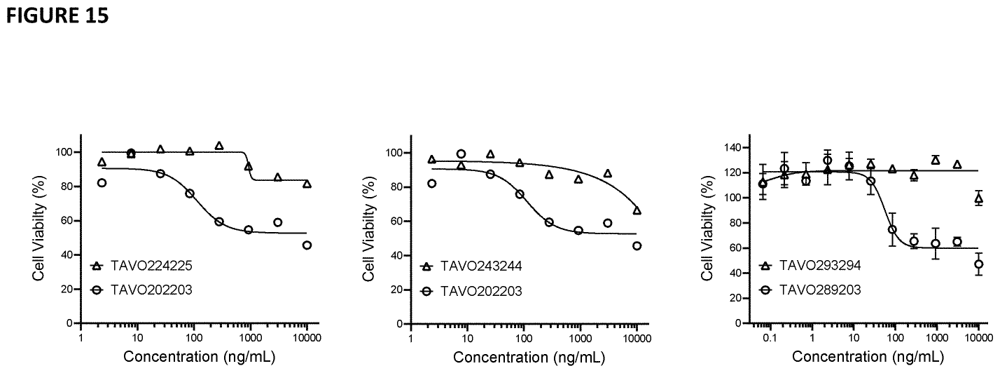

An in vitro cell proliferation assay was set up to evaluate anti-HER2 shielded antibody in inhibition of the proliferation of HER2-expressing cell, breast cancer cell line SK-BR-3. Increasing amounts of anti-HER2 shielded antibodies TAVO224225, TAVO243244 or TAVO293294 were applied to the SK-BR-3 cells for six days and their effects on cell proliferation were assessed by a cell proliferation detection kit. The anti-HER2 shielded antibodies with IGF2-based masking domain (TAVO224225, TAVO243244 or TAVO293294) showed minimal inhibition of SK-BR-3 cell proliferation up to 10 μg/mL (FIG. 15), while their counterpart anti-HER2 antibodies without the IGF2 mask (TAVO202203 and TAVO289203) showed potent inhibition of SK-BR-3 cell proliferation.

### Example 14: Effector Functions of Shielded Anti-HER2 Antibody

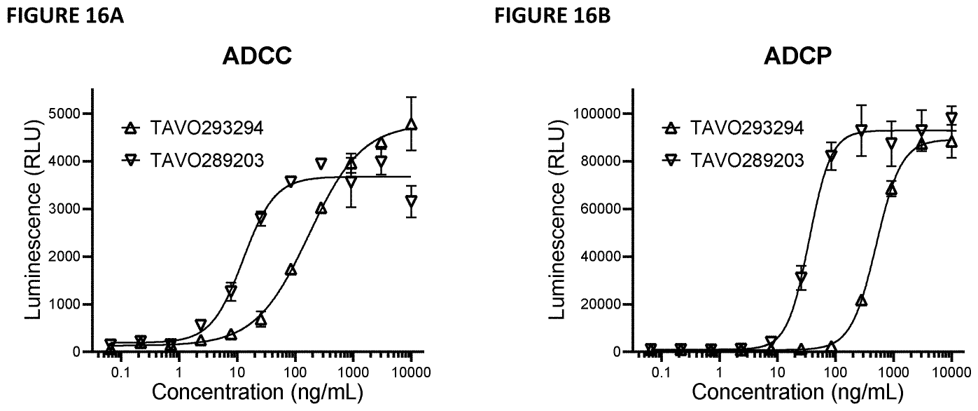

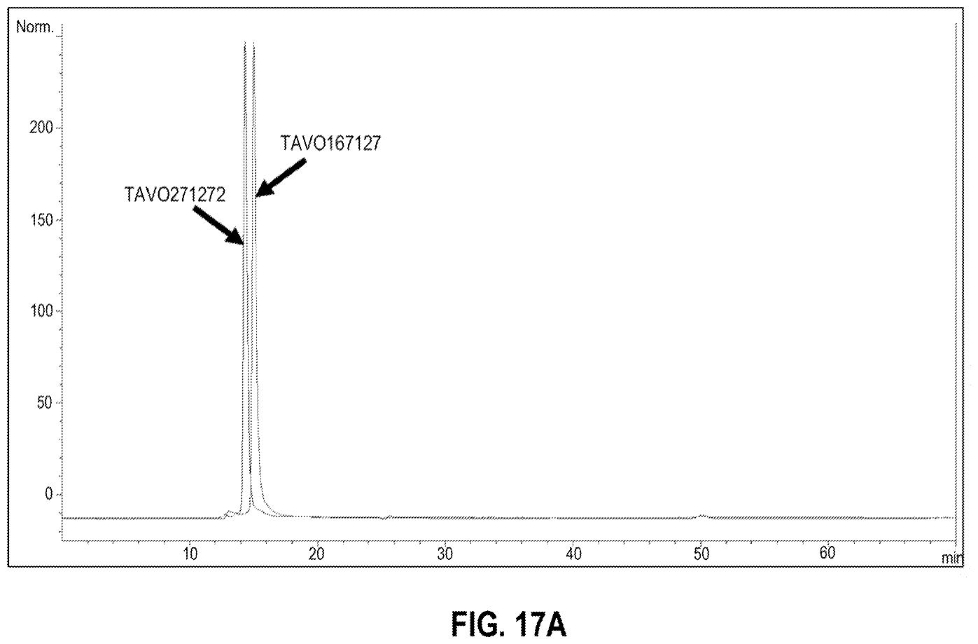

An ADCC reporter assay (Invivogen) was set up to assess an anti-HER2 shielded antibody in the activation of ADCC. Increasing amounts of anti-HER2 shielded antibody TAVO293294 was incubated with ADCC reporter cells along with HER2-expressing SK-BR-3 cells. The antibody-mediated ADCC activity was measured by luciferase gene expression in ADCC reporter cells (Invivogen). The shielded anti-HER2 antibody TAVO293294 with IGF2-based masking domain showed 10-fold lower potency in mediating ADCC activity than its counterpart anti-HER2 antibody TAVO289203 without the IGF2 mask (FIG. 16A).

An ADCP reporter assay (Invivogen) was set up to assess shielded anti-HER2 antibody in the activation of ADCP. Increasing amounts of shielded anti-HER2 antibody TAVO293294 was incubated with ADCP reporter cells along with HER2-expressing SK-BR-3 cells. The antibody-mediated ADCP activity was measured by luciferase gene expression in ADCP reporter cells (Invivogen). The shielded anti-HER2 antibody TAVO293294 with IGF2-based masking domain showed 10-fold lower potency in mediating ADCP activity than its counterpart anti-HER2 antibody TAVO289203 without the IGF2 mask (FIG. 16B).

### Example 15: In Vivo Anti-Tumor Efficacy of Anti-HER2 Shielded Antibody with IGF2-Based Masking Domain

The efficacy of anti-HER2 shielded antibody with IGF2-based masking domain in tumor cell killing will be evaluated in a mouse tumor xenograft model. HER2-expressing cells, such as breast cancer cell line BT-474 and SK-BR-3 and gastric cancer cell line NCL-N87, will be inoculated in nude mice for tumor establishment. Anti-HER2 shielded antibody will be intraperitoneally administered to the mice and the antibody-mediated tumor shrinkage will be assessed. The anti-HER2 shielded antibody with IGF2-based masking domain is expected to have comparable anti-tumor activity to the active anti-HER2 antibody owing to the overexpression of proteases needed to convert anti-HER2 shielded antibody into active antibody at the tumor site.

### Example 16: Cardiotoxicity of Anti-HER2 Shielded Antibody with IGF2-Based Masking Domain

High dose administration of anti-HER2 antibody to patients may increase the risk of cardiac dysfunction, including heart failure, due to the presence of HER2 in cardiomyocytes. In order to show anti-HER2 shielded antibody with IGF2-based masking domain may have reduced risk of cardiotoxicity, an ex vivo cardiotoxicity model will be established. Anti-HER2 antibody and shielded antibody with IGF2-based masking domain will be incubated with human induced pluripotent stem cell derived cardiomyocyte (iPSC-CM) and their effects on the viability of iPSC-CM will be assessed. It is expected that the shielded antibody by TavoPRECISE-Shield with IGF2-based masking domain will show much less effect on iPSC-CM viability due to the masking of anti-HER2 antibody activity by the masking domain.

Further exemplary embodiments are illustrated below.
1. A shielded biologic comprising:
   a) a heavy chain polypeptide comprising, from N-terminus to C-terminus, a first masking domain unit, a first protease-cleavable linker, and an antibody heavy chain or an antigen-binding fragment thereof; and
   b) a light chain polypeptide comprising, from N-terminus to C-terminus, a second masking domain unit, a second protease-cleavable linker, and an antibody light chain or an antigen-binding fragment thereof, wherein the first masking domain unit and the second masking domain unit form a masking domain.
2. The shielded biologic of embodiment 1, wherein the first masking domain unit comprises a sequence based on the A chain or B chain of Insulin Growth Factor 2 (IGF2), and the second masking domain unit comprises a sequence based on the B chain or A chain of IGF2.
3. The shielded biologic of any one of embodiments 1-2, wherein the masking domain is capable of shielding the antigen binding capability of the biologics in normal tissues, and wherein in target disease tissues, one or both of the masking domain units are cleaved off by disease site-specific proteases and the shielded biologic is converted to an active antibody.
4. The shielded biologic of any one of embodiments 1-3, wherein the shielded biologic comprises a heavy chain polypeptide and a light chain polypeptide selected from the heavy chain and light chain sequences listed in Tables 2, 3, 4, and 5.
5. The shielded biologic of any one of embodiments 1-3, wherein the shielded biologic comprises a combination of a heavy chain and a light chain with an amino acid sequence having at least 80% identity (e.g., at least 80%, 81%, 82%, 83%, 84%, 85%, 86%, 87%, 88%, 89%, 90%, 91%, 92%, 93%, 94%, 95%, 96%, 97%, 98%, or 99%) to the respective heavy chain and light chain amino acid sequences chosen from SEQ ID NO: 1 to 16, 41 to 52, 55 to 64, and 73 to 81.
6. The shielded biologic of any one of embodiments 1-3, wherein the first and second masking domain units comprise sequences derived from human IGF2 pre-prohormone comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) identity to the amino acid sequence set forth as SEQ ID NO: 17.
7. The shielded biologic of any one of embodiments 1-3, wherein the first or second masking domain unit comprises a human IGF2 A chain sequence comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) amino acid identity to the acid sequence set forth as SEQ ID NO: 18, and the first or second masking domain unit comprises a human IGF2 B chain sequence comprising an amino acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) amino acid identity to the sequence set forth as SEQ ID NO: 19, and wherein the A chain and B chain of IGF2 forms a heterodimeric complex through two interchain disulfide bonds.
8. The shielded biologic of any one of embodiments 1-3, wherein the first or second masking domain unit comprises human IGF2 A chain or B chain with mutations chosen from SEQ ID NO: 69-72, wherein the mutations disrupt IGF2 binding to its receptor.
9. The shielded biologic of any one of embodiments 1-3, wherein the first or second masking domain unit comprises human IGF2 A chain with a V43L mutation set forth as SEQ ID NO: 70 or human IGF2 B chain with a Y27A mutation set forth as SEQ ID NO: 71, wherein the mutations disrupt IGF2 binding to its receptor.
10. The shielded biologic of embodiment 1, wherein the first and second masking domain units comprise sequences derived from pre-prohormone sequences of a member of Insulin-Relaxin superfamily, comprising amino acid sequences having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) identity to the amino acid sequence chosen from SEQ ID NO: 21 to 29.
11. The shielded biologic of any one of embodiments 1-10, wherein the first and second protease-cleavable linkers comprise a protease substrate sequence for a protease enriched in target disease site chosen from matrix metalloprotease, Cathepsin, urokinase plasminogen activator (uPA), disintegrin and metalloproteinase, wherein the first and second protease-cleavable linkers may comprise the same or different protease substrate sequences.
12. The shielded biologic of any one of embodiments 1-11, wherein the first and/or second protease-cleavable linkers comprise a substrate sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) identity to the amino acid sequence chosen from SEQ ID NO: 30 to 40.
13. The shielded biologic of any one of embodiments 1-12, wherein the shielded biologic is against one or more therapeutic targets chosen from, but are not limited to, TNFα, IL-1β, HER2, VEGF, EGFR, cMET, Nectin-4, CTLA-4, CD3ε, a4-integrin, CD20, CD11a, CD52, RANK-L, PD-1, PD-L1, CD47, CD24, CD166, and CD71.
14. The shielded biologic of any one of embodiments 1-13, wherein the shielded biologic is a monoclonal antibody, bispecific antibody, or multi-specific antibody with IgG, IgA, IgD, IgM subtypes.
15. The shielded biologic of any one of embodiments 1-13, wherein the shielded biologic comprises an antigen-binding fragment, or an antigen-binding fragment in antibody drug conjugate format, or an antigen-binding fragment in a chimeric antigen receptor format, or an antigen binding fragment on a non-antibody scaffold.
16. The shielded biologic of any one of embodiments 1-14, wherein the heavy chain polypeptide has one or more F_{c} mutations that extend the half-life of the shielded biologic when compared to the native shielded biologic without the one or mutations.
17. The shielded biologic of any one of embodiments 1-14, wherein the heavy chain polypeptide has one or more sets of mutations selected from M252Y/S254T/T256E, M428L/N434S, T250Q/M428L, N434A and T307A/E380A/N434A when compared to the native shielded biologic without the mutations, according to the EU Index residue numbering.
18. The shielded biologics of any one of embodiments 1-14, wherein the heavy chain polypeptide has one or more F_{c} mutations that enhance the resistance of the shielded biologics to proteolytic degradation by a protease that cleaves the native shielded biologic without the one or more mutations between or at residues 222-237, according to the EU Index residue numbering.
19. The shielded biologics of any one of embodiments 1-14, wherein the heavy chain polypeptide comprises E233P/L234A/L235A Fc mutations with G236 deleted when compared to the native shielded biologic without the mutations, residue numbering according to the EU Index residue numbering.
20. The shielded biologic of any one of embodiments 1-14, wherein the heavy chain polypeptide has one or more F_{c} mutations that reduce or eliminate the effector functions of engineered shielded biologic compared to the native shielded biologic without the one or more mutations.
21. The shielded biologic of any one of embodiments 1-14, wherein the heavy chain polypeptide has L234A, L235A, M428L, and N434S F_{c} mutations that extend the half-life and reduce the effector functions of the engineered shielded biologic, residue numbering according to the EU Index, compared to the shielded biologic.
22. The shielded biologic of any one of embodiments 1-14, wherein the heavy chain polypeptide has E233P, L234A, L235A, M428L, and N434S F_{c} mutations with G236 deleted that extend the half-life, reduce the effector functions, and enhance the resistance of the shielded biologic to proteolytic degradation by a protease, residue numbering according to the EU Index, compared to the native shielded biologic.
23. The shielded biologic of any one of embodiments 1-14, wherein the heavy chain polypeptide has one or more F_{c} mutations chosen from an F405L mutation and a K409R mutation, wherein the one or more mutations can facilitate heavy chain heterodimerization when compared to the native shielded biologic without the one or more mutations, residue numbering according to the EU Index.
24. An isolated polynucleotide encoding the shielded biologic of any one of embodiments 1-23.
25. A vector comprising the polynucleotide of embodiment 24.
26. The vector of embodiment 25, which is an expression vector.
27. A host cell comprising the vector of embodiment 25 or 26.
28. A pharmaceutical composition comprising the shielded biologic of any one of embodiments 1-23 and a pharmaceutical carrier.
29. A method of producing the shielded biologic of any one of embodiments 1-23, comprising culturing the host cell of embodiment 27 in conditions wherein the shielded biologic is expressed, and isolating the shielded biologic.
30. A method of measuring the half-life of the shielded biologic of any one of embodiments 1-23.
31. A method of measuring the resistance to proteolytic degradation of the shielded biologic of any one of embodiments 1-23.
32. A method of measuring the effector functions of the shielded biologic by of any one of embodiments 1-23.
33. A method of cleaving the first and/or the second masking domain unit off the shielded biologic of any one of embodiments 1-23, by one or more proteases, and converting the shielded biologic into an active antibody.
34. A method of detecting the cleavage of the first and/or the second masking domain unit from the shielded biologic of any one of embodiments 1-23, by one or more proteases.
35. A method of measuring the antigen-binding capability of the shielded biologic of any one of embodiments 1-23 and the antibody after the first and/or the second masking domain unit is cleaved off by one or more proteases.
36. A method of measuring the functional activities of the shielded biologic of any one of embodiments 1-23 and the antibody after the first and/or the second masking domain unit is cleaved off by one or more proteases by in vitro assays.
37. A method of measuring the in vivo efficacy of the shielded biologic of any one of embodiments 1-23 and the antibody after the first and/or the second masking domain unit is cleaved off by one or more proteases by one or more animal drug potency studies.
38. A method of measuring the safety profile of the shielded biologic of any one of embodiments 1-23 and the antibody after the first and/or the second masking domain unit is cleaved off by one or more proteases by one or more animal toxicity models.
39. A method for treating an auto-immune and/or inflammatory disease in a subject in need thereof, comprising administering to the subject an effective amount of the shielded biologic of any one of embodiments 1-23 and/or the pharmaceutical composition of embodiment 28, wherein the disease is chosen from rheumatoid arthritis, systemic lupus erythematosus, osteoarthritis, ankylosing spondylitis, Behcet's Disease, gout, psoriatic arthritis, multiple sclerosis, Crohn's colitis, and inflammatory bowel disease.
40. A method for treating a condition in a subject in need thereof, comprising administering to the subject an effective amount of the shielded biologic of any one of embodiments 1-23 and/or the pharmaceutical composition of embodiment 28, wherein the condition is chosen from Type II diabetes mellitus, Parkinson's disease, age-related macular degeneration, polyneuropathy, sensory peripheral neuropathy, proliferative diabetic retinopathy, diabetic neuropathy, decubitus ulcer, fulminant Type 1 diabetes, retinal vasculitis, non-infectious posterior uveitis, and alcoholic neuropathy.
41. A method for treating cancer in a subject in need thereof, comprising administering to the subject an effective amount of the shielded biologic of any one of embodiments 1-23 and/or the pharmaceutical composition of embodiment 28, wherein the cancer is chosen from multiple myeloma, non-small cell lung cancer, acute myeloid leukemia, female breast cancer, pancreatic cancer, colorectal cancer and peritoneum cancer.
42. The method of embodiments 39 to 41, wherein said administering is subcutaneous.
43. The method of embodiments 39 to 41, wherein said administering is intravenous.
44. The method of embodiments 39 to 41, wherein said administering is intramuscular.
45. The method of embodiments 39 to 41, wherein said administering is oral or rectal.
46. The method of embodiments 39 to 41, wherein said administering is systemic.
47. The method of embodiments 39 to 41, wherein said administering is local.

### Sequences

Provided herein is a representative list of certain sequences included in embodiments provided herein.

TABLE 5
Sequences

| SEQ | | |
|---|---|---|
| ID | Description | Sequence |
| SEQ | anti-TNFα shielded | GIVEECCFRS |
| ID | antibody by | CDLALLETYC |
| NO: | TavoPRECISE-Shield | AGGGGSPLGL |
| 1 | heavy chain EAC190 | AGGGGGSEVQ |
| | based on ADA-H2X | LVESGGGLVQ |
| | variable domain with | PGGSLRLSCA |
| | IGF2 A chain | ASGFDFADYA |
| | (underlined), | MHWVRQAPGK |
| | MMP2/9 substrate | GLEWVSAITW |
| | sequence (bolded) and | NGGHTDYADS |
| | IgG1 Fc with E233P, | VKGRFTISRD |
| | L234A, L235A, | NSKNTLYLQM |
| | F405L, M428L, | NSLRAEDTAV |
| | N434S mutations and | YYCAKVSYLS |
| | G236 deleted | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSK |
| | | LTVDKSRWQQ |
| | | GNVFSCSVLH |
| | | EALHSHYTQK |
| | | SLSLSPGK |
| SEQ | anti-TNFα shielded | AYRPSETLCG |
| ID | antibody by | GELVDTLQFV |
| NO: | TavoPRECISE-Shield | CGDRGFYFGG |
| 2 | light chain EAC191 | GGSPLGLAGG |
| | based on ADA-L1 | GGGSEIVMTQ |
| | variable domain with | SPATLSVSPG |
| | IGF2 B chain | ERATLSCRAS |
| | (underlined) and | QGIRNYLAWY |
| | MMP2/9 substrate | QQKPGQAPRL |
| | sequence (bolded) | LIYAASTLQS |
| | | GIPARFSGSG |
| | | SGTEFTLTIS |
| | | SLQSEDFAVY |
| | | YCQRYNRAPY |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-TNFα shielded | GIVEECCFRS |
| ID | antibody by | CDLALLETYC |
| NO: | TavoPRECISE-Shield | AGGGGSRPKP |
| 3 | heavy chain EAC196 | VEVWRKGGGG |
| | based on ADA-H2X | SEVQLVESGG |
| | variable domain with | GLVQPGGSLR |
| | IGF2 A chain | LSCAASGFDF |
| | (underlined), MMP3 | ADYAMHWVRQ |
| | substrate sequence | APGKGLEWVS |
| | (bolded) and IgG1 Fc | AITWNGGHTD |
| | with E233P, L234A, | YADSVKGRFT |
| | L235A, F405L, | ISRDNSKNTL |
| | M428L, N434S | YLQMNSLRAE |
| | mutations and G236 | DTAVYYCAKV |
| | deleted | SYLSTASSLD |
| | | YWGQGTLVTV |
| | | SSASTKGPSV |
| | | FPLAPSSKST |
| | | SGGTAALGCL |
| | | VKDYFPEPVT |
| | | VSWNSGALTS |
| | | GVHTFPAVLQ |
| | | SSGLYSLSSV |
| | | VTVPSSSLGT |
| | | QTYICNVNHK |
| | | PSNTKVDKKV |
| | | EPKSCDKTHT |
| | | CPPCPAPPAA |
| | | GPSVFLFPPK |
| | | PKDTLMISRT |
| | | PEVTCVVVDV |
| | | SHEDPEVKFN |
| | | WYVDGVEVHN |
| | | AKTKPREEQY |
| | | NSTYRVVSVL |
| | | TVLHQDWLNG |
| | | KEYKCKVSNK |
| | | ALPAPIEKTI |
| | | SKAKGQPREP |
| | | QVYTLPPSRD |
| | | ELTKNQVSLT |
| | | CLVKGFYPSD |
| | | IAVEWESNGQ |
| | | PENNYKTTPP |
| | | VLDSDGSFLL |
| | | YSkLTVDKSR |
| | | WQQGNVFSCS |
| | | VLHEALHSHY |
| | | TQKSLSLSPG |
| | | K |
| SEQ | anti-TNFα shielded | AYRPSETLCG |
| ID | antibody by | GELVDTLQFV |
| NO: | TavoPRECISE-Shield | CGDRGFYFGG |
| 4 | light chain EAC197 | GGSRPKPVEV |
| | based on ADA-L1 | WRKGGGGSEI |
| | variable domain with | VMTQSPATLS |
| | IGF2 B chain | VSPGERATLS |
| | (underlined) and | CRASQGIRNY |
| | MMP3 substrate | LAWYQQKPGQ |
| | sequence (bolded) | APRLLIYAAS |
| | | TLQSGIPARF |
| | | SGSGSGTEFT |
| | | LTISSLQSED |
| | | FAVYYCQRYN |
| | | RAPYTFGQGT |
| | | KVEIKRTVAA |
| | | PSVFIFPPSD |
| | | EQLKSGTASV |
| | | VCLLNNFYPR |
| | | EAKVQWKVDN |
| | | ALQSGNSQES |
| | | VTEQDSKDST |
| | | YSLSSTLTLS |
| | | KADYEKHKVY |
| | | ACEVTHQGLS |
| | | SPVTKSFNRG |
| | | EC |
| SEQ | anti-IL-1β shielded | GIVEECCFRS |
| ID | antibody by | CDLALLETYC |
| NO: | TavoPRECISE-Shield | AGGGGSPLGL |
| 5 | heavy chain EAC212 | AGGGGGSEVQ |
| | with IGF2 A chain | LVESGGGLVQ |
| | (underlined), | PGGSLRLSCA |
| | MMP2/9 substrate | ASGFTFSSFG |
| | sequence (bolded) and | MHWVRQAPGK |
| | IgG1 Fc with E233P, | GLEWVAYISI |
| | L234A, L235A, | GSYTVHYADS |
| | K409R, M428L, | VKGRFTISRD |
| | N434S mutations and | NAKNSLYLQM |
| | G236 deleted | NSLRDEDTAV |
| | | YYCVRDDYDV |
| | | TDYTMDYWGQ |
| | | GTTVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFFLYSRL |
| | | TVDKSRWQQG |
| | | NVFSCSVLHE |
| | | ALHSHYTQKS |
| | | LSLSPGK |
| SEQ | anti-IL-1β shielded | AYRPSETLCG |
| ID | antibody by | GELVDTLQFV |
| NO: | TavoPRECISE-Shield | CGDRGFYFGG |
| 6 | light chain EAC213 | GGSPLGLAGG |
| | with IGF2 B chain | GGGSDIVMTQ |
| | (underlined) and | TPLSLPVTPG |
| | MMP2/9 substrate | EPASISCKSS |
| | sequence (bolded) | QSLLNSRTRK |
| | | NYLAWYLQKP |
| | | GQSPQLLIYW |
| | | ASTRESGVPD |
| | | RFSGSGSGTD |
| | | FTLKISRVEA |
| | | EDVGVYYCKQ |
| | | TYNFPTFGQG |
| | | TKLEIKRTVA |
| | | APSVFIFPPS |
| | | DEQLKSGTAS |
| | | VVCLLNNFYP |
| | | REAKVQWKVD |
| | | NALQSGNSQE |
| | | SVTEQDSKDS |
| | | TYSLSSTLTL |
| | | SKADYEKHKV |
| | | YACEVTHQGL |
| | | SSPVTKSFNR |
| | | GEC |
| SEQ | anti-IL-1β shielded | GIVEECCFRS |
| ID | antibody by | CDLALLETYC |
| NO: | TavoPRECISE-Shield | AGGGGSRPKP |
| 7 | heavy chain EAC214 | VEVWRKGGGG |
| | with IGF2 A chain | SEVQLVESGG |
| | (underlined), MMP3 | GLVQPGGSLR |
| | substrate sequence | LSCAASGFTF |
| | (bolded) and IgG1 Fc | SSFGMHWVRQ |
| | with E233P, L234A, | APGKGLEWVA |
| | L235A, K409R, | YISIGSYTVH |
| | M428L, N434S | YADSVKGRFT |
| | mutations and G236 | ISRDNAKNSL |
| | deleted | YLQMNSLRDE |
| | | DTAVYYCVRD |
| | | DYDVTDYTMD |
| | | YWGQGTTVTV |
| | | SSASTKGPSV |
| | | FPLAPSSKST |
| | | SGGTAALGCL |
| | | VKDYFPEPVT |
| | | VSWNSGALTS |
| | | GVHTFPAVLQ |
| | | SSGLYSLSSV |
| | | VTVPSSSLGT |
| | | QTYICNVNHK |
| | | PSNTKVDKKV |
| | | EPKSCDKTHT |
| | | CPPCPAPPAA |
| | | GPSVFLFPPK |
| | | PKDTLMISRT |
| | | PEVTCVVVDV |
| | | SHEDPEVKFN |
| | | WYVDGVEVHN |
| | | AKTKPREEQY |
| | | NSTYRVVSVL |
| | | TVLHQDWLNG |
| | | KEYKCKVSNK |
| | | ALPAPIEKTI |
| | | SKAKGQPREP |
| | | QVYTLPPSRD |
| | | ELTKNQVSLT |
| | | CLVKGFYPSD |
| | | IAVEWESNGQ |
| | | PENNYKTTPP |
| | | VLDSDGSFFL |
| | | YSRLTVDKSR |
| | | WQQGNVFSCS |
| | | VLHEALHSHY |
| | | TQKSLSLSPG |
| | | K |
| SEQ | anti-IL-1β shielded | AYRPSETLCG |
| ID | antibody by | GELVDTLQFV |
| NO: | TavoPRECISE-Shield | CGDRGFYFGG |
| 8 | light chain EAC215 | GGSRPKPVEV |
| | with IGF2 B chain | WRKGGGGSDI |
| | (underlined) and | VMTQTPLSLP |
| | MMP3 substrate | VTPGEPASIS |
| | sequence (bolded) | CKSSQSLLNS |
| | | RTRKNYLAWY |
| | | LQKPGQSPQL |
| | | LIYWASTRES |
| | | GVPDRFSGSG |
| | | SGTDFTLKIS |
| | | RVEAEDVGVY |
| | | YCKQTYNFPT |
| | | FGQGTKLEIK |
| | | RTVAAPSVFI |
| | | FPPSDEQLKS |
| | | GTASVVCLLN |
| | | NFYPREAKVQ |
| | | WKVDNALQSG |
| | | NSQESVTEQD |
| | | SKDSTYSLSS |
| | | TLTLSKADYE |
| | | KHKVYACEVT |
| | | HQGLSSPVTK |
| | | SFNRGEC |
| SEQ | anti-HER2 shielded | GIVEECCFRS |
| ID | antibody by | CDLALLETYC |
| NO: | TavoPRECISE-Shield | AGGGGSPLGL |
| 9 | heavy chain EAC224 | AGGGGGSEVQ |
| | with 1GF2 A chain | LVESGGGLVQ |
| | (underlined), | PGGSLRLSCA |
| | MMP2/9 substrate | ASGFNIKDTY |
| | sequence (bolded) and | IHWVRQAPGK |
| | IgG1 Fc with E233P, | GLEWVARIYP |
| | F405L, M428L, | TNGYTRYADS |
| | N434S mutations and | VKGRFTISAD |
| | G236 deleted | TSKNTAYLQM |
| | | NSLRAEDTAV |
| | | YYCSRWGGDG |
| | | FYAMDYWGQG |
| | | TLVTVSSAST |
| | | KGPSVFPLAP |
| | | SSKSTSGGTA |
| | | ALGCLVKDYF |
| | | PEPVTVSWNS |
| | | GALTSGVHTF |
| | | PAVLQSSGLY |
| | | SLSSVVTVPS |
| | | SSLGTQTYIC |
| | | NVNHKPSNTK |
| | | VDKKVEPKSC |
| | | DKTHTCPPCP |
| | | APPLLGPSVF |
| | | LFPPKPKDTL |
| | | MISRTPEVTC |
| | | VVVDVSHEDP |
| | | EVKFNWYVDG |
| | | VEVHNAKTKP |
| | | REEQYNSTYR |
| | | VVSVLTVLHQ |
| | | DWLNGKEYKC |
| | | KVSNKALPAP |
| | | IEKTISKAKG |
| | | QPREPQVYTL |
| | | PPSRDELTKN |
| | | QVSLTCLVKG |
| | | FYPSDIAVEW |
| | | ESNGQPENNY |
| | | KTTPPVLDSD |
| | | GSFLLYSkLT |
| | | VDKSRWQQGN |
| | | VFSCSVLHEA |
| | | LHSHYTQKSL |
| | | SLSPGK |
| SEQ | anti-HER2 shielded | AYRPSETLCG |
| ID | antibody by | GELVDTLQFV |
| NO: | TavoPRECISE-Shield | CGDRGFYFGG |
| 10 | light chain EAC225 | GGSPLGLAGG |
| | with IGF2 B chain | GGGSDIQMTQ |
| | (underlined) and | SPSSLSASVG |
| | MMP2/9 substrate | DRVTITCRAS |
| | sequence (bolded) | QDVNTAVAWY |
| | | QQKPGKAPKL |
| | | LIYSASFLYS |
| | | GVPSRFSGSR |
| | | SGTDFTLTIS |
| | | SLQPEDFATY |
| | | YCQQHYTTPP |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-HER2 shielded | GIVEECCFRS |
| ID | antibody by | CDLALLETYC |
| NO: | TavoPRECISE-Shield | AGGGGSLSGR |
| 11 | heavy chain EAC243 | SDNHGGGGSE |
| | with IGF2 A chain | VQLVESGGGL |
| | (underlined), uPA | VQPGGSLRLS |
| | substrate sequence | CAASGFNIKD |
| | (bolded) and IgG1 Fc | TYIHWVRQAP |
| | with E233P, F405L, | GKGLEWVARI |
| | M428L, N434S | YPTNGYTRYA |
| | mutations and G236 | DSVKGRFTIS |
| | deleted | ADTSKNTAYL |
| | | QMNSLRAEDT |
| | | AVYYCSRWGG |
| | | DGFYAMDYWG |
| | | QGTLVTVSSA |
| | | STKGPSVFPL |
| | | APSSKSTSGG |
| | | TAALGCLVKD |
| | | YFPEPVTVSW |
| | | NSGALTSGVH |
| | | TFPAVLQSSG |
| | | LYSLSSVVTV |
| | | PSSSLGTQTY |
| | | ICNVNHKPSN |
| | | TKVDKKVEPK |
| | | SCDKTHTCPP |
| | | CPAPPLLGPS |
| | | VFLFPPKPKD |
| | | TLMISRTPEV |
| | | TCVVVDVSHE |
| | | DPEVKFNWYV |
| | | DGVEVHNAKT |
| | | KPREEQYNST |
| | | YRVVSVLTVL |
| | | HQDWLNGKEY |
| | | KCKVSNKALP |
| | | APIEKTISKA |
| | | KGQPREPQVY |
| | | TLPPSRDELT |
| | | KNQVSLTCLV |
| | | KGFYPSDIAV |
| | | EWESNGQPEN |
| | | NYKTTPPVLD |
| | | SDGSFLLYSK |
| | | LTVDKSRWQQ |
| | | GNVFSCSVLH |
| | | EALHSHYTQK |
| | | SLSLSPGK |
| SEQ | anti-HER2 shielded | AYRPSETLCG |
| ID | antibody by | GELVDTLQFV |
| NO: | TavoPRECISE-Shield | CGDRGFYFGG |
| 12 | light chain EAC244 | GGSLSGRSDN |
| | with IGF2 B chain | HGGGGSDIQM |
| | (underlined) and uPA | TQSPSSLSAS |
| | substrate sequence | VGDRVTITCR |
| | (bolded) | ASQDVNTAVA |
| | | WYQQKPGKAP |
| | | KLLIYSASFL |
| | | YSGVPSRFSG |
| | | SRSGTDFTLT |
| | | ISSLQPEDFA |
| | | TYYCQQHYTT |
| | | PPTFGQGTKV |
| | | EIKRTVAAPS |
| | | VFIFPPSDEQ |
| | | LKSGTASVVC |
| | | LLNNFYPREA |
| | | KVQWKVDNAL |
| | | QSGNSQESVT |
| | | EQDSKDSTYS |
| | | LSSTLTLSKA |
| | | DYEKHKVYAC |
| | | EVTHQGLSSP |
| | | VTKSFNRGEC |
| SEQ | anti-VEGF shielded | GIVEECCFRS |
| ID | antibody by | CDLALLETYC |
| NO: | TavoPRECISE-Shield | AGGGGSPLGL |
| 13 | heavy chain EAC259 | AGGGGGSEVQ |
| | with IGF2 A chain | LVESGGGLVQ |
| | (underlined), | PGGSLRLSCA |
| | MMP2/9 substrate | ASGYTFTNYG |
| | sequence (bolded) and | MNWVRQAPGK |
| | IgGl Fc with E233P, | GLEWVGWINT |
| | K409R, M428L, | YTGEPTYAAD |
| | N434S mutations and | FKRRFTFSLD |
| | G236 deleted | TSKSTAYLQM |
| | | NSLRAEDTAV |
| | | YYCAKYPHYY |
| | | GSSHWYFDVW |
| | | GQGTLVTVSS |
| | | ASTKGPSVFP |
| | | LAPSSKSTSG |
| | | GTAALGCLVK |
| | | DYFPEPVTVS |
| | | WNSGALTSGV |
| | | HTFPAVLQSS |
| | | GLYSLSSVVT |
| | | VPSSSLGTQT |
| | | YICNVNHKPS |
| | | NTKVDKKVEP |
| | | KSCDKTHTCP |
| | | PCPAPPLLGP |
| | | SVFLFPPKPK |
| | | DTLMISRTPE |
| | | VTCVVVDVSH |
| | | EDPEVKFNWY |
| | | VDGVEVHNAK |
| | | TKPREEQYNS |
| | | TYRVVSVLTV |
| | | LHQDWLNGKE |
| | | YKCKVSNKAL |
| | | PAPIEKTISK |
| | | AKGQPREPQV |
| | | YTLPPSRDEL |
| | | TKNQVSLTCL |
| | | VKGFYPSDIA |
| | | VEWESNGQPE |
| | | NNYKTTPPVL |
| | | DSDGSFFLYS |
| | | RLTVDKSRWQ |
| | | QGNVFSCSVL |
| | | HEALHSHYTQ |
| | | KSLSLSPGK |
| SEQ | anti-VEGF shielded | AYRPSETLCG |
| ID | antibody by | GELVDTLQFV |
| NO: | TavoPRECISE-Shield | CGDRGFYFGG |
| 14 | light chain EAC229 | GGSPLGLAGG |
| | with IGF2 B chain | GGGSDIQMTQ |
| | (underlined) and | SPSSLSASVG |
| | MMP2/9 substrate | DRVTITCSAS |
| | sequence (bolded) | QDISNYLNWY |
| | | QQKPGKAPKV |
| | | LIYFTSSLHS |
| | | GVPSRFSGSG |
| | | SGTDFTLTIS |
| | | SLQPEDFATY |
| | | YCQQYSTVPW |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-VEGF shielded | GIVEECCFRS |
| ID | antibody by | CDLALLETYC |
| NO: | TavoPRECISE-Shield | AGGGGSLSGR |
| 15 | heavy chain EAC245 | SDNHGGGGSE |
| | with IGF2 A chain | VQLVESGGGL |
| | (underlined), uPA | VQPGGSLRLS |
| | substrate sequence | CAASGYTFTN |
| | (bolded) and IgGl Fc | YGMNWVRQAP |
| | with E233P, F405L, | GKGLEWVGWI |
| | M428L, N434S | NTYTGEPTYA |
| | mutations and G236 | ADFKRRFTFS |
| | deleted | LDTSKSTAYL |
| | | QMNSLRAEDT |
| | | AVYYCAKYPH |
| | | YYGSSHWYFD |
| | | VWGQGTLVTV |
| | | SSASTKGPSV |
| | | FPLAPSSKST |
| | | SGGTAALGCL |
| | | VKDYFPEPVT |
| | | VSWNSGALTS |
| | | GVHTFPAVLQ |
| | | SSGLYSLSSV |
| | | VTVPSSSLGT |
| | | QTYICNVNHK |
| | | PSNTKVDKKV |
| | | EPKSCDKTHT |
| | | CPPCPAPPLL |
| | | GPSVFLFPPK |
| | | PKDTLMISRT |
| | | PEVTCVVVDV |
| | | SHEDPEVKFN |
| | | WYVDGVEVHN |
| | | AKTKPREEQY |
| | | NSTYRVVSVL |
| | | TVLHQDWLNG |
| | | KEYKCKVSNK |
| | | ALPAPIEKTI |
| | | SKAKGQPREP |
| | | QVYTLPPSRD |
| | | ELTKNQVSLT |
| | | CLVKGFYPSD |
| | | IAVEWESNGQ |
| | | PENNYKTTPP |
| | | VLDSDGSFLL |
| | | YSkLTVDKSR |
| | | WQQGNVFSCS |
| | | VLHEALHSHY |
| | | TQKSLSLSPG |
| | | K |
| SEQ | anti-VEGF shielded | AYRPSETLCG |
| ID | antibody by | GELVDTLQFV |
| NO: | TavoPRECISE-Shield | CGDRGFYFGG |
| 16 | light chain EAC246 | GGSLSGRSDN |
| | with IGF2 B chain | HGGGGSDIQM |
| | (underlined) and uPA | TQSPSSLSAS |
| | substrate sequence | VGDRVTITCS |
| | (bolded) | ASQDISNYLN |
| | | WYQQKPGKAP |
| | | KVLIYFTSSL |
| | | HSGVPSRFSG |
| | | SGSGTDFTLT |
| | | ISSLQPEDFA |
| | | TYYCQQYSTV |
| | | PWTFGQGTKV |
| | | EIKRTVAAPS |
| | | VFIFPPSDEQ |
| | | LKSGTASVVC |
| | | LLNNFYPREA |
| | | KVQWKVDNAL |
| | | QSGNSQESVT |
| | | EQDSKDSTYS |
| | | LSSTLTLSKA |
| | | DYEKHKVYAC |
| | | EVTHQGLSSP |
| | | VTKSFNRGEC |
| SEQ | Human IGF2 pre- | MGIPMGKSML |
| ID | prohormonc | VLLTFLAFAS |
| NO: | | CCIAAYRPSE |
| 17 | | TLCGGELVDT |
| | | LQFVCGDRGF |
| | | YFSRPASRVS |
| | | RRSRGIVEEC |
| | | CFRSCDLALL |
| | | ETYCATPAKS |
| | | ERDVSTPPTV |
| | | LPDNFPRYPV |
| | | GKFFQYDTWK |
| | | QSTQRLRRGL |
| | | PALLRARRGH |
| | | VLAKELEAFR |
| | | EAKRHRPLIA |
| | | LPTQDPAHGG |
| | | APPEMASNRK |
| SEQ | Human IGF2 A chain | GIVEECCFRS |
| ID | | CDLALLETYC |
| NO: | | A |
| 18 | | |
| SEQ | Human IGF2 B chain | AYRPSETLCG |
| ID | | GELVDTLQFV |
| NO: | | CGDRGFYF |
| 19 | | |
| SEQ | Human IGF2 C chain | SRPASRVSRR |
| ID | | SR |
| NO: | | |
| 20 | | |
| SEQ | Human Insulin pre- | MALWMRLLPL |
| ID | prohormonc | LALLALWGPD |
| NO: | | PAAAFVNQHL |
| 21 | | CGSHLVEALY |
| | | LVCGERGFFY |
| | | TPKTRREAED |
| | | LQVGQVELGG |
| | | GPGAGSLQPL |
| | | ALEGSLQKRG |
| | | IVEQCCTSIC |
| | | SLYQLENYCN |
| SEQ | Human IGF1 pre- | MGKISSLPTQ |
| ID | prohormonc | LFKCCFCDFL |
| NO: | | KVKMHTMSSS |
| 22 | | HLFYLALCLL |
| | | TFTSSATAGP |
| | | ETLCGAELVD |
| | | ALQFVCGDRG |
| | | FYFNKPTGYG |
| | | SSSRRAPQTG |
| | | IVDECCFRSC |
| | | DLRRLEMYCA |
| | | PLKPAKSARS |
| | | VRAQRHTDMP |
| | | KTQKYQPPST |
| | | NKNTKSQRRK |
| | | GWPKTHPGGE |
| | | QKEGTEASLQ |
| | | IRGKKKEQRR |
| | | EIGSRNAECR |
| | | GKKGK |
| SEQ | Human Relaxin-1 prc- | MPRLFLFHLL |
| ID | prohormonc | EFCLLLNQFS |
| NO: | | RAVAAKWKDD |
| 23 | | VIKLCGRELV |
| | | RAQIAICGMS |
| | | TWSKRSLSQE |
| | | DAPQTPRPVA |
| | | EIVPSFINKD |
| | | TETIIIMLEF |
| | | IANLPPELKA |
| | | ALSERQPSLP |
| | | ELQQYVPALK |
| | | DSNLSFEEFK |
| | | KLIRNRQSEA |
| | | ADSNPSELKY |
| | | LGLDTHSQKK |
| | | RRPYVALFEK |
| | | CCLIGCTKRS |
| | | LAKYC |
| SEQ | Human Relaxin-2 pre- | MPRLFFFHLL |
| ID | prohormone | GVCLLLNQFS |
| NO: | | RAVADSWMEE |
| 24 | | VIKLCGRELV |
| | | RAQIAICGMS |
| | | TWSKRSLSQE |
| | | DAPQTPRPVA |
| | | EIVPSFINKD |
| | | TETINMMSEF |
| | | VANLPQELKL |
| | | TLSEMQPALP |
| | | QLQQHVPVLK |
| | | DSSLLFEEFK |
| | | KLIRNRQSEA |
| | | ADSSPSELKY |
| | | LGLDTHSRKK |
| | | RQLYSALANK |
| | | CCHVGCTKRS |
| | | LARFC |
| SEQ | Human Relaxin-3 pre- | MARYMLLLLL |
| ID | prohormone | AVWVLTGELW |
| NO: | | PGAEARAAPY |
| 25 | | GVRLCGREFI |
| | | RAVIFTCGGS |
| | | RWRRSDILAH |
| | | EAMGDTFPDA |
| | | DADEDSLAGE |
| | | LDEAMGSSEW |
| | | LALTKSPQAF |
| | | YRGRPSWQGT |
| | | PGVLRGSRDV |
| | | LAGLSSSCCK |
| | | WGCSKSEISS |
| | | LC |
| SEQ | Human INSL3 prc- | MDPRLPAWAL |
| ID | prohormonc | VLLGPALVFA |
| NO: | | LGPAPTPEMR |
| 26 | | EKLCGHHFVR |
| | | ALVRVCGGPR |
| | | VVSTEARRPA |
| | | TGGDRELLQW |
| | | LERRHLLHGL |
| | | VADSNLTLGP |
| | | GLQPLPQTSH |
| | | HHRHHRAAAT |
| | | NPARYCCLSG |
| | | CTQQDLLTLC |
| | | PY |
| SEQ | Human INSL4 prc- | MASLFRSYLP |
| ID | prohormonc | AIWLLLSQLL |
| NO: | | RESLAAELRG |
| 27 | | CGPRFGKHLL |
| | | SYCPMPEKTF |
| | | TTTPGGWLLE |
| | | SGRPKEMVST |
| | | SNNKDGQALG |
| | | TTSEFIPNLS |
| | | PELKKPLSEG |
| | | QPSLKKIILS |
| | | RKKRSGRHRF |
| | | DPFCCEVICD |
| | | DGTSVKLCT |
| SEQ | Human INSL5 prc- | MKGSIFTLFL |
| ID | prohormonc | FSVLFAISEV |
| NO: | | RSKESVRLCG |
| 28 | | LEYIRTVIYI |
| | | CASSRWRRHQ |
| | | EGIPQAQQAE |
| | | TGNSFQLPHK |
| | | REFSEENPAQ |
| | | NLPKVDASGE |
| | | DRLWGGQMPT |
| | | EELWKSKKHS |
| | | VMSRQDLQTL |
| | | CCTDGCSMTD |
| | | LSALC |
| SEQ | Human INSL6 prc- | MPRLLRLSLL |
| ID | prohormone | WLGLLLVRFS |
| NO: | | RELSDISSAR |
| 29 | | KLCGRYLVKE |
| | | IEKLCGHANW |
| | | SQFRFEEETP |
| | | FSRLIAQASE |
| | | KVEAYSPYQF |
| | | ESPQTASPAR |
| | | GRGTNPVSTS |
| | | WEEAVNSWEM |
| | | QSLPEYKDKK |
| | | GYSPLGK |
| | | TREFSSSHNI |
| | | NVYIHENAKF |
| | | QKKRRNKIKT |
| | | LSNLFWGHHP |
| | | QRKRRGYSEK |
| | | CCLTGCTKEE |
| | | LSIACLPYID |
| | | FKRLKEKRSS |
| | | LVTKIY |
| SEQ | MMP2/9 protcase- | GGGGSGPLGV |
| 10 | cleavable linker | RGGGGS |
| NO: | sequence | |
| 30 | | |
| SEQ | uPA protease- | GGGGSLSGRS |
| ID | cleavable linker | DNHGGGGS |
| NO: | sequence | |
| 31 | | |
| SEQ | MMP3 protease- | GGGGSRPKPV |
| ID | cleavable linker | EVWRKGGGGS |
| NO: | sequence | |
| 32 | | |
| SEQ | Substrate sequence | GPLGVR |
| ID | for MMP2 and | |
| NO: | MMP9 | |
| 33 | | |
| SEQ | Substrate sequence | PLGLAR |
| ID | for MMP2 and | |
| NO: | MMP9 | |
| 34 | | |
| SEQ | Substrate sequence | PLGLAG |
| II) | for MMP2 and | |
| NO: | MMP9 | |
| 35 | | |
| SEQ | Substrate sequence | IPVSLRSG |
| ID | for MMP2 and | |
| NO: | MMP9 | |
| 36 | | |
| SEQ | Substrate sequence | GPLGMLSQ |
| ID | for MMP2 and | |
| NO: | MMP9 | |
| 37 | | |
| SEQ | Substrate sequence | RPKPVEVWRK |
| ID | for MMP3 | |
| NO: | | |
| 38 | | |
| SEQ | Substrate sequence | LSGRSDNH |
| ID | for uPA | |
| NO: | | |
| 39 | | |
| SEQ | Substrate sequence | TGRGPSWV |
| ID | for uPA | |
| NO: | | |
| 40 | | |
| SEQ | anti-TNFα shielded | GILEECCFRS |
| ID | antibody by | CDLALLETYC |
| NO: | TavoPRECISE-Shield | AGGGGSPLGL |
| 41 | heavy chain with | AGGGGGSEVQ |
| | IGF2 A chain with | LVESGGGLVQ |
| | V43L mutation | P |
| | (underlined), | GRSLRLSCAA |
| | MMP2/9 substrate | SGFTFDDYAM |
| | sequence (bolded) and | HWVRQAPGKG |
| | Adalimumab heavy | LEWVSAITWN |
| | chain | SGHIDYADSV |
| | | EGRFTISRDN |
| | | AKNSLYLQMN |
| | | SLRAEDTAVY |
| | | YCAKVSYLST |
| | | ASSLDYWGQG |
| | | TLVTVSSAST |
| | | KGPSVFPLAP |
| | | SSKSTSGGTA |
| | | ALGCLVKDYF |
| | | PEPVTVSWNS |
| | | GALTSGVHTF |
| | | PAVLQSSGLY |
| | | SLSSWTVPSS |
| | | SLGTQTYICN |
| | | VNHKPSNTKV |
| | | DKKVEPKSCD |
| | | KTHTCPPCPA |
| | | PELLGGPSVF |
| | | LFPPKPKDTL |
| | | MISRTPEVTC |
| | | VVVDVSHEDP |
| | | EVKFNWYVDG |
| | | VEVHNAKTKP |
| | | REEQYNSTYR |
| | | VVSVLTVLHQ |
| | | DWLNGKEYKC |
| | | KVSNKALPAP |
| | | IEKTISKAKG |
| | | QPREPQVYTL |
| | | PPSRDELTKN |
| | | QVSLTCLVKG |
| | | FYPSDIAVEW |
| | | ESNGQPENNY |
| | | KTTPPVLDSD |
| | | GSFFLYSkLT |
| | | VDKSRWQQGN |
| | | VFSCSVMHEA |
| | | LHNHYTQKSL |
| | | SLSPGK |
| SEQ | anti-TNFα shielded | AYRPSETLCG |
| ID | antibody by | GELVDTLQFV |
| NO: | TavoPRECISE-Shield | CGDRGFAFGG |
| 42 | light chain with IGF2 | GGSPLGLAGG |
| | B chain with Y27A | GGGSDIQMTQ |
| | mutation (underlined), | SPSSLSASVG |
| | MMP2/9 substrate | DRVTITCRAS |
| | sequence (bolded), | QGIRNYLAWY |
| | and Adalimumab light | QQKPGKAPKL |
| | chain | LIYAASTLQS |
| | | GVPSRFSGSG |
| | | SGTDFTLTIS |
| | | SLQPEDVATY |
| | | YCQRYNRAPY |
| | | TFGQGTKVEI |
| | | KRTVAAPSVF |
| | | IFPPSDEQLK |
| | | SGTASWCLLN |
| | | NFYPREAKVQ |
| | | WKVDNALQSG |
| | | NSQESVTEQD |
| | | SKDSTYSLSS |
| | | TLTLSKADYE |
| | | KHKVYACEVT |
| | | HQGLSSPVTK |
| | | SFNRGEC |
| SEQ | anti-TNFα shielded | GILEECCFRS |
| ID | antibody by | CDLALLETYC |
| NO: | TavoPRECISE-Shield | AGGGGSRPKP |
| 43 | heavy chain with | VEVWRKGGGG |
| | IGF2 A chain with | SEVQLVESGG |
| | V43L mutation | GLVQPGGSLR |
| | (underlined), MMP3 | LSCAASGFDF |
| | substrate sequence | ADYAMHWVRQ |
| | (bolded) and | APGKGLEWVS |
| | Infliximab heavy | AITWNGGHTD |
| | chain | YADSVKGRFT |
| | | ISRDNSKNTL |
| | | YLQMNSLRAE |
| | | DTAVYYCAKV |
| | | SYLSTASSLD |
| | | YWGQGTLVTV |
| | | SSASTKGPSV |
| | | FPLAPSSKST |
| | | SGGTAALGCL |
| | | VKDYFPEPVT |
| | | VSWNSGALTS |
| | | GVHTFPAVLQ |
| | | SSGLYSLSSV |
| | | VTVPSSSLGT |
| | | QTYICNVNHK |
| | | PSNTKVDKKV |
| | | EPKSCDKTHT |
| | | CPPCPAPELL |
| | | GGPSVFLFPP |
| | | KPKDTLMISR |
| | | TPEVTCVVVD |
| | | VSHEDPEVKF |
| | | NWYVDGVEVH |
| | | NAKTKPREEQ |
| | | YNSTYRVVSV |
| | | LTVLHQDWLN |
| | | GKEYKCKVSN |
| | | KALPAPIEKT |
| | | ISKAKGQPRE |
| | | PQVYTLPPSR |
| | | DELTKNQVSL |
| | | TCLVKGFYPS |
| | | DIAVEWESNG |
| | | QPENNYKTTP |
| | | PVLDSDGSFF |
| | | LYSkLTVDKS |
| | | RWQQGNVFSC |
| | | SVMHEALHNH |
| | | YTQKSLSLSP |
| | | GK |
| SEQ | anti-TNFα shielded | AYRPSETLCG |
| ID | antibody by | GELVDTLQFV |
| NO: | TavoPRECISE-Shield | CGDRGFAFGG |
| 44 | light chain with IGF2 | GGSRPKPVEV |
| | B chain with Y27A | WRKGGGGSEI |
| | mutation (underlined), | VMTQSPATLS |
| | MMP3 substrate | VSPGERATLS |
| | sequence (bolded) and | CRASQGIRNY |
| | Infliximab light chain | LAWYQQKPGQ |
| | | APRLLIYAAS |
| | | TLQSGIPARF |
| | | SGSGSGTEFT |
| | | LTISSLQSED |
| | | FAVYYCQRYN |
| | | RAPYTFGQGT |
| | | KVEIKRTVAA |
| | | PSVFIFPPSD |
| | | EQLKSGTASV |
| | | VCLLNNFYPR |
| | | EAKVQWKVDN |
| | | ALQSGNSQES |
| | | VTEQDSKDST |
| | | YSLSSTLTLS |
| | | KADYEKHKVY |
| | | ACEVTHQGLS |
| | | SPVTKSFNRG |
| | | EC |
| SEQ | anti-IL-1β shielded | GILEECCFRS |
| ID | antibody by | CDLALLETYC |
| NO: | TavoPRECISE-Shield | AGGGGSPLGL |
| 45 | heavy chain with | AGGGGGSQVQ |
| | IGF2 A chain with | LVESGGGVVQ |
| | V43L mutation | PGRSLRLSCA |
| | (underlined), | ASGFTFSVYG |
| | MMP2/9 substrate | MNWVRQAPGK |
| | sequence (bolded) and | GLEWVAIIWY |
| | Canakinumab heavy | DGDNQYYADS |
| | chain | VKGRFTISRD |
| | | NSKNTLYLQM |
| | | NGLRAEDTAV |
| | | YYCARDLRTG |
| | | PFDYWGQGTL |
| | | VTVSSASTKG |
| | | PSVFPLAPSS |
| | | KSTSGGTAAL |
| | | GCLVKDYFPE |
| | | PVTVSWNSGA |
| | | LTSGVHTFPA |
| | | VLQSSGLYSL |
| | | SSWTVPSSSL |
| | | GTQTYICNVN |
| | | HKPSNTKVDK |
| | | RVEPKSCDKT |
| | | HTCPPCPAPE |
| | | LLGGPSVFLF |
| | | PPKPKDTLMI |
| | | SRTPEVTCVW |
| | | DVSHEDPEVK |
| | | FNWYVDGVEV |
| | | HNAKTKPREE |
| | | QYNSTYRVVSV |
| | | LTVLHQDWLN |
| | | GKEYKCKVSN |
| | | KALPAPIEKT |
| | | ISKAKGQPRE |
| | | PQVYTLPPSR |
| | | EEMTKNQVSL |
| | | TCLVKGFYPS |
| | | DIAVEWESNG |
| | | QPENNYKTTP |
| | | PVLDSDGSFF |
| | | LYSkLTVDKS |
| | | RWQQGNVFSC |
| | | SVMHEALHNH |
| | | YTQKSLSLSP |
| | | GK |
| SEQ | anti-IL-1β shielded | AYRPSETLCG |
| ID | antibody by | GELVDTLQFV |
| NO: | TavoPRECISE-Shield | CGDRGFAFGG |
| 46 | light chain with IGF2 | GGSPLGLAGG |
| | B chain with Y27A | GGGSEIVLTQ |
| | mutation (underlined), | SPDFQSVTPK |
| | MMP2/9 substrate | EKVTITCRAS |
| | sequence (bolded), | QSIGSSLHWY |
| | and Canakinumab | QQKPDQSPKL |
| | light chain | LIKYASQSFS |
| | | GVPSRFSGSG |
| | | SGTDFTLTIN |
| | | SLEAEDAAAY |
| | | YCHQSSSLPF |
| | | TFGPGTKVDI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-EGFR shielded | GILEECCFRS |
| ID | antibody by | CDLALLETYC |
| NO: | TavoPRECISE-Shield | AGGGGSPLGL |
| 47 | heavy chain with | AGGGGGSQVQ |
| | IGF2 A chain with | LKQSGPGLVQ |
| | V43L mutation | PSQSLSITCT |
| | (underlined), | VSGFSLTNYG |
| | MMP2/9 substrate | VHWVRQSPGK |
| | sequence (bolded) | GLEWLGVIWS |
| | and | GGNTDYNTPF |
| | Cetuximab heavy | TSRLSINKDN |
| | chain | SKSQVFFKMN |
| | | SLQSNDTAIY |
| | | YCARALTYYD |
| | | YEFAYWGQGT |
| | | LVTVSAASTK |
| | | GPSVFPLAPS |
| | | SKSTSGGTAA |
| | | LGCLVKDYFP |
| | | EPVTVSWNSG |
| | | ALTSGVHTFP |
| | | AVLQSSGLYS |
| | | LSSVVTVPSS |
| | | SLGTQTYICN |
| | | VNHKPSNTKV |
| | | DKKVEPKSCD |
| | | KTHTCPPCPA |
| | | PELLGGPSVF |
| | | LFPPKPKDTL |
| | | MISRTPEVTC |
| | | VVVDVSHEDP |
| | | EVKFNWYVDG |
| | | VEVHNAKTKP |
| | | REEQYNSTYR |
| | | WSVLTVLHQD |
| | | WLNGKEYKCK |
| | | VSNKALPAPI |
| | | EKTISKAKGQ |
| | | PREPQVYTLP |
| | | PSRDELTKNQ |
| | | VSLTCLVKGF |
| | | YPSDIAVEWE |
| | | SNGQPENNYK |
| | | TTPPVLDSDG |
| | | SFFLYSkLTV |
| | | DKSRWQQGNV |
| | | FSCSVMHEAL |
| | | HNHYTQKSLS |
| | | LSPGK |
| SEQ | anti-EGFR shielded | AYRPSETLCG |
| ID | antibody by | GELVDTLQFV |
| NO: | TavoPRECISE-Shield | CGDRGFAFGG |
| 48 | light chain with IGF2 | GGSPLGLAGG |
| | B chain with Y27A | GGGSDILLTQ |
| | mutation (underlined), | SPVILSVSPG |
| | MMP2/9 substrate | ERVSFSCRAS |
| | sequence (bolded), | QSIGTNIHWY |
| | and Cetuximab light | QQRTNGSPRL |
| | chain | LIKYASESIS |
| | | GIPSRFSGSG |
| | | SGTDFTLSIN |
| | | SVESEDIADY |
| | | YCQQNNNWPT |
| | | TFGAGTKLEL |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-CTLA-4 shielded | GILEECCFRS |
| ID | antibody by | CDLALLETYC |
| NO: | TavoPRECISE-Shield | AGGGGSLSGR |
| 49 | heavy chain with | SDNHGGGGSQ |
| | IGF2 A chain with | VQLVESGGGV |
| | V43L mutation | VQPGRSLRLS |
| | (underlined), uPA | CAASGFTFSS |
| | substrate sequence | YTMHWVRQAP |
| | (bolded) and | GKGLEWVTFI |
| | Ipilimumab heavy | SYDGNNKYYA |
| | chain | DSVKGRFTIS |
| | | RDNSKNTLYL |
| | | QMNSLRAEDT |
| | | AIYYCARTGW |
| | | LGPFDYWGQG |
| | | TLVTVSSAST |
| | | KGPSVFPLAP |
| | | SSKSTSGGTA |
| | | ALGCLVKDYF |
| | | PEPVTVSWNS |
| | | GALTSGVHTF |
| | | PAVLQSSGLY |
| | | SLSSWTVPSS |
| | | SLGTQTYICN |
| | | VNHKPSNTKV |
| | | DKRVEPKSCD |
| | | KTHTCPPCPA |
| | | PELLGGPSVF |
| | | LFPPKPKDTL |
| | | MISRTPEVTC |
| | | VWDVSHEDPE |
| | | VKFNWYVDGV |
| | | EVHNAKTKPR |
| | | EEQYNSTYRW |
| | | SVLTVLHQDW |
| | | LNGKEYKCKV |
| | | SNKALPAPIE |
| | | KTISKAKGQP |
| | | REPQVYTLPP |
| | | SRDELTKNQV |
| | | SLTCLVKGFY |
| | | PSDIAVEWES |
| | | NGQPENNYKT |
| | | TPPVLDSDGS |
| | | FFLYSkLTVD |
| | | KSRWQQGNVF |
| | | SCSVMHEALH |
| | | NHYTQKSLSL |
| | | SPGK |
| SEQ | anti-CTLA-4 shielded | AYRPSETLCG |
| ID | antibody by | GELVDTLQFV |
| NO: | TavoPRECISE-Shield | CGDRGFAFGG |
| 50 | light chain with IGF2 | GGSLSGRSDN |
| | B chain with Y27A | HGGGGSEIVL |
| | mutation (underlined), | TQSPGTLSLS |
| | uPA substrate | PGERATLSCR |
| | sequence (bolded), | ASQSVGSSYL |
| | and Ipilimumab light | AWYQQKPGQA |
| | chain | PRLLIYGAFS |
| | | RATGIPDRFS |
| | | GSGSGTDFTL |
| | | TISRLEPEDF |
| | | AVYYCQQYGS |
| | | SPWTFGQGTK |
| | | VEIKRTVAAP |
| | | SVFIFPPSDE |
| | | QLKSGTASVV |
| | | CLLNNFYPRE |
| | | AKVQWKVDNA |
| | | LQSGNSQESV |
| | | TEQDSKDSTY |
| | | SLSSTLTLSK |
| | | ADYEKHKVYA |
| | | CEVTHQGLSS |
| | | PVTKSFNRGE |
| | | C |
| SEQ | Shielded anti-CD3e | GILEECCFRS |
| ID | heavy chain with | CDLALLETYC |
| NO: | IGF2 A chain with | AGGGGSPLGL |
| 51 | V43L mutation | AGGGGGSQVQ |
| | (underlined), | LQQSGAELAR |
| | MMP2/9 substrate | PGASVKMSCK |
| | sequence (bolded) and | ASGYTFTRYT |
| | Muromonab vH | MHWVKQRPGQ |
| | sequence | GLEWIGYINP |
| | | SRGYTNYNQK |
| | | FKDKATLTTD |
| | | KSSSTAYMQL |
| | | SSLTSEDSAV |
| | | YYCARYYDDH |
| | | YCLDYWGQGT |
| | | TLTVSS |
| SEQ | Shielded anti-CD3e | AYRPSETLCG |
| ID | light chain with IGF2 | GELVDTLQFV |
| NO: | B chain with Y27A | CGDRGFAFGG |
| 52 | mutation (underlined), | GGSPLGLAGG |
| | MMP2/9 substrate | GGGSQIVLTQ |
| | sequence (bolded) and | SPAIMSASPG |
| | Muromonab vL | EKVTMTCSAS |
| | sequence | SSVSYMNWYQ |
| | | QKSGTSPKRW |
| | | IYDTSkLASG |
| | | VPAHFRGSGS |
| | | GTSYSLTISG |
| | | MEAEDAATYY |
| | | CQQWSSNPFT |
| | | FGSGTKLEI |
| SEQ | Nucleic acid sequence | GGCATCGTGG |
| ID | encoding heavy chain | AGGAGTGCTG |
| NO: | polypeptide EAC190 | CTTCAGAAGC |
| 53 | | TGCGATCTGG |
| | | CTCTGCTGGA |
| | | GACCTACTGC |
| | | GCTGGAGGAG |
| | | GCGGCAGCCC |
| | | CCTGGGACTG |
| | | GCTGGCGGAG |
| | | GAGGAGGCAG |
| | | CGAGGTGCAG |
| | | CTGGTGGAGA |
| | | GCGGAGGAGG |
| | | ACTGGTGCAG |
| | | CCCGGCGGCT |
| | | CTCTGAGACT |
| | | GAGCTGTGCC |
| | | GCCAGCGGCT |
| | | TCGACTTTGC |
| | | CGACTACGCC |
| | | ATGCACTGGG |
| | | TGAGACAAGC |
| | | CCCCGGCAAA |
| | | GGACTGGAGT |
| | | GGGTGAGCGC |
| | | CATCACATGG |
| | | AACGGCGGCC |
| | | ACACAGACTA |
| | | CGCCGATAGC |
| | | GTGAAGGGAA |
| | | GATTCACCAT |
| | | CTCTAGAGAC |
| | | AACAGCAAGA |
| | | ACACACTGTA |
| | | TCTGCAGATG |
| | | AACTCTCTGA |
| | | GAGCCGAGGA |
| | | CACCGCCGTG |
| | | TACTACTGCG |
| | | CCAAGGTGAG |
| | | CTATCTGAGC |
| | | ACCGCCAGCT |
| | | CTCTGGACTA |
| | | CTGGGGACAA |
| | | GGCACACTGG |
| | | TGACCGTGTC |
| | | CTCCGCCA |
| | | GCACCAAGGG |
| | | CCCAAGCGTG |
| | | TTTCCTCTGG |
| | | CCCCCTCCTC |
| | | CAAAAGCACA |
| | | TCCGGCGGAA |
| | | CAGCCGCCCT |
| | | CGGATGTCTG |
| | | GTCAAGGACT |
| | | ACTTCCCCGA |
| | | ACCCGTGACA |
| | | GTCAGCTGGA |
| | | ATAGCGGCGC |
| | | CCTCACAAGC |
| | | GGCGTGCACA |
| | | CATTTCCCGC |
| | | CGTGCTCCAG |
| | | AGCAGCGGAC |
| | | TGTATTCTCT |
| | | GTCCTCCGTG |
| | | GTCACAGTCC |
| | | CCAGCTCCTC |
| | | CCTCGGCACC |
| | | CAGACCTATA |
| | | TTTGCAACGT |
| | | GAACCATAAG |
| | | CCCTCCAACA |
| | | CCAAAGTCGA |
| | | CAAGAAGGTG |
| | | GAGCCCAAGA |
| | | GCTGCGATAA |
| | | GACCCATACA |
| | | TGCCCCCCTT |
| | | GTCCCGCCCC |
| | | CCCTGCTGCT |
| | | GGTCCCAGCG |
| | | TGTTTTTATT |
| | | TCCTCCTAAG |
| | | CCCAAGGACA |
| | | CTTTAATGAT |
| | | CTCTCGTACC |
| | | CCCGAGGTGA |
| | | CATGTGTGGT |
| | | GGTGGACGTC |
| | | TCCCATGAGG |
| | | ACCCCGAGGT |
| | | GAAGTTCAAT |
| | | TGGTACGTCG |
| | | ACGGCGTGGA |
| | | GGTGCACAAT |
| | | GCCAAGACCA |
| | | AGCCTAGAGA |
| | | GGAGCAGTAC |
| | | AACTCCACCT |
| | | ATAGGGTGGT |
| | | GTCCGTGCTC |
| | | ACCGTGCTGC |
| | | ACCAAGATTG |
| | | GCTGAACGGC |
| | | AAGGAGTACA |
| | | AGTGTAAGGT |
| | | GTCCAACAAG |
| | | GCTCTGCCCG |
| | | CTCCCATCGA |
| | | GAAGACCATC |
| | | TCCAAGGCCA |
| | | AAGGCCAGCC |
| | | TAGAGAACCC |
| | | CAAGTGTATA |
| | | CACTGCCCCC |
| | | TAGOAGAGAT |
| | | GAACTGACCA |
| | | AGAACCAAGT |
| | | GTCTCTGACA |
| | | TGTCTGGTCA |
| | | AGGGATTCTA |
| | | CCCCAGCGAC |
| | | ATCGCCGTGG |
| | | AGTGGGAGAG |
| | | CAATGGCCAG |
| | | CCCGAAAACA |
| | | ACTACAAGAC |
| | | CACCCCTCCC |
| | | GTGCTCGACA |
| | | GCGACGGATC |
| | | CTTTCTCCTG |
| | | TACAGCAAGC |
| | | TGACCGTGGA |
| | | CAAAAGCAGA |
| | | TGGCAGCAAG |
| | | GCAACGTCTT |
| | | TAGCTGCAGC |
| | | GTGCTGCACG |
| | | AGGCTCTGCA |
| | | TAGCCACTAC |
| | | ACACAGAAGT |
| | | CTCTGTCTCT |
| | | GTCCCCCGGC |
| | | AAG |
| SEQ | Nucleic acid sequence | GCCTACAGAC |
| ID | encoding light chain | CCAGCGAGAC |
| NO: | polypeptide EAC191 | ACTGTGTGGC |
| 54 | | GGCGAGCTGG |
| | | TGGACACACT |
| | | GCAGTTCGTG |
| | | TGCGGCGACA |
| | | GAGGCTTCTA |
| | | CTTCGGCGGC |
| | | GGAGGCAGCC |
| | | CCCTGGGACT |
| | | GGCTGGAGGC |
| | | GGAGGCGGAA |
| | | GCGAGATCGT |
| | | GATGACCCAG |
| | | AGCCCCGCCA |
| | | CACTGAGCGT |
| | | GAGCCCCGGC |
| | | GAGAGGGCCA |
| | | CACTGAGCTG |
| | | CAGAGCCAGC |
| | | CAAGGCATCA |
| | | GAAACTATCT |
| | | GGCTTGGTAC |
| | | CAGCAGAAGC |
| | | CCGGCCAAGC |
| | | CCCCAGACTG |
| | | CTGATCTACG |
| | | CCGCCAGCAC |
| | | ACTGCAGAGC |
| | | GGCATTCCCG |
| | | CCAGATTCAG |
| | | CGGAAGCGGC |
| | | AGCGGAACCG |
| | | AGTTCACACT |
| | | GACCATCTCC |
| | | TCTCTGCAGA |
| | | GCGAGGACTT |
| | | CGCCGTGTAC |
| | | TACTGCCAGA |
| | | GGTACAACAG |
| | | AGCCCCCTAC |
| | | ACCTTCGGCC |
| | | AAGGCACCAA |
| | | GGTGGAGATC |
| | | AAGCGTACGG |
| | | TGGCTGCACC |
| | | ATCTGTCTTC |
| | | ATCTTCCCGC |
| | | CATCTGATGA |
| | | GCAGTTGAAA |
| | | TCTGGAACTG |
| | | CCTCTGTTGT |
| | | GTGCCTGCTG |
| | | AATAACTTCT |
| | | ATCCCAGAGA |
| | | GGCCAAAGTA |
| | | CAGTGGAAGG |
| | | TGGATAACGC |
| | | CCTCCAATCG |
| | | GGTAACTCCC |
| | | AGGAGAGTGT |
| | | CACAGAGCAG |
| | | GACAGCAAGG |
| | | ACAGCACCTA |
| | | CAGCCTCAGC |
| | | AGCACCCTGA |
| | | CGCTGAGCAA |
| | | AGCAGACTAC |
| | | GAGAAACACA |
| | | AAGTCTACGC |
| | | CTGCGAAGTC |
| | | ACCCATCAGG |
| | | GCCTGAGCTC |
| | | GCCCGTCACA |
| | | AAGAGCTTCA |
| | | ACAGGGGAGA |
| | | GTGT |
| SEQ | anti-TNFα shielded | GIVEECCFRS |
| TD | antibody by | CDLALLETYC |
| NO: | TavoPRECISE-Shield | AGGGGSGPLG |
| 55 | heavy chain EAC186 | VRGGGGSEVQ |
| | based on ADA-H2X | LVESGGGLVQ |
| | variable domain with | PGGSLRLSCA |
| | IGF2 A chain | ASGFDFADYA |
| | (underlined), | MHWVRQAPGK |
| | MMP2/9 substrate | GLEWVSAITW |
| | sequence (bolded) and | NGGHTDYADS |
| | IgG1 Fc with E233P, | VKGRFTISRD |
| | L234A, L235A, | NSKNTLYLQM |
| | F405L, M428L, | NSLRAEDTAV |
| | N434S mutations and | YYCAKVSYLS |
| | G236 deleted | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQG |
| | | NVFSCSVLHE |
| | | ALHSHYTQKS |
| | | LSLSPGK |
| SEQ | anti-TNFα shielded | AYRPSETLCG |
| ID | antibody by | GELVDTLQFV |
| NO: | TavoPRECISE-Shield | CGDRGFYFGG |
| 56 | light chain EAC187 | GGSGPLGVRG |
| | based on ADA-L1 | GGGSEIVMTQ |
| | variable domain with | SPATLSVSPG |
| | IGF2 B chain | ERATLSCRAS |
| | (underlined) and | QGIRNYLAWY |
| | MMP2/9 substrate | QQKPGQAPRL |
| | sequence (bolded) | LIYAASTLQS |
| | | GIPARFSGSG |
| | | SGTEFTLTIS |
| | | SLQSEDFAVY |
| | | YCQRYNRAPY |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-TNFα shielded | GIVEECCFRS |
| ID | antibody by | CDLALLETYC |
| NO: | TavoPRECISE-Shield | AGGGGSPLGL |
| 57 | heavy chain EAC188 | ARGGGGSEVQ |
| | based on ADA-H2X | LVESGGGLVQ |
| | variable domain with | PGGSLRLSCA |
| | IGF2 A chain | ASGFDFADYA |
| | (underlined), | MHWVRQAPGK |
| | MMP2/9 substrate | GLEWVSAITW |
| | sequence (bolded) and | NGGHTDYADS |
| | IgG1 Fc with E233P, | VKGRFTISRD |
| | L234A, L235A, | NSKNTLYLQM |
| | F405L, M428L, | NSLRAEDTAV |
| | N434S mutations and | YYCAKVSYLS |
| | G236 deleted | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQG |
| | | NVFSCSVLHE |
| | | ALHSHYTQKS |
| | | LSLSPGK |
| SEQ | anti-TNFα shielded | AYRPSETLCG |
| ID | antibody by | GELVDTLQFV |
| NO: | TavoPRECISE-Shield | CGDRGFYFGG |
| 58 | light chain EAC189 | GGSPLGLARG |
| | based on ADA-L1 | GGGSEIVMTQ |
| | variable domain with | SPATLSVSPG |
| | IGF2 B chain | ERATLSCRAS |
| | (underlined) and | QGIRNYLAWY |
| | MMP2/9 substrate | QQKPGQAPRL |
| | sequence (bolded) | LIYAASTLQS |
| | | GIPARFSGSG |
| | | SGTEFTLTIS |
| | | SLQSEDFAVY |
| | | YCQRYNRAPY |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-TNFα shielded | GIVEECCFRS |
| ID | antibody by | CDLALLETYC |
| NO: | TavoPRECISE-Shield | AGGGGSIPVS |
| 59 | heavy chain EAC192 | LRSGGGGGSE |
| | based on ADA-H2X | VQLVESGGGL |
| | variable domain with | VQ |
| | IGF2 A chain | PGGSLRLSCA |
| | (underlined), | ASGFDFADYA |
| | MMP2/9 substrate | MHWVRQAPGK |
| | sequence (bolded) and | GLEWVSAITW |
| | IgG1 Fc with E233P, | NGGHTDYADS |
| | L234A, L235A, | VKGRFTISRD |
| | F405L, M428L, | NSKNTLYLQM |
| | N434S mutations and | NSLRAEDTAV |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQG |
| | | NVFSCSVLH |
| | | EALHSHYTQK |
| | | SLSLSPGK |
| SEQ | anti-TNFα shielded | AYRPSETLCG |
| ID | antibody by | GELVDTLQFV |
| NO: | TavoPRECISE-Shield | CGDRGFYFGG |
| 60 | light chain EAC193 | GGSIPVSLRS |
| | based on ADA-L1 | GGGGGSEIVM |
| | variable domain with | TQSPATLSVS |
| | IGF2 B chain | PGERATLSCR |
| | (underlined) and | ASQGIRNYLA |
| | MMP2/9 substrate | WYQQKPGQAP |
| | sequence (bolded) | RLLIYAASTL |
| | | QSGIPARFSG |
| | | SGSGTEFTLT |
| | | ISSLQSEDFA |
| | | VYYCQRYNRA |
| | | PYTFGQGTKV |
| | | EIKRTVAAPS |
| | | VFIFPPSDEQ |
| | | LKSGTASVVC |
| | | LLNNFYPREA |
| | | KVQWKVDNAL |
| | | QSGNSQESVT |
| | | EQDSKDSTYS |
| | | LSSTLTLSKA |
| | | DYEKHKVYAC |
| | | EVTHQGLSSP |
| | | VTKSFNRGEC |
| SEQ | anti-TNFα shielded | GIVEECCFRS |
| ID | antibody by | CDLALLETYC |
| NO: | TavoPRECISE-Shield | AGGGGSGPLG |
| 61 | heavy chain EAC194 | MLSQGGGGSE |
| | based on ADA-H2X | VQLVESGGGL |
| | variable domain with | VQPGGSLRLS |
| | IGF2 A chain | CAASGFDFAD |
| | (underlined), | YAMHWVRQAP |
| | MMP2/9 substrate | GKGLEWVSAI |
| | sequence (bolded) and | TWNGGHTDYA |
| | IgG1 Fc with E233P, | DSVKGRFTIS |
| | L234A, L235A, | RDNSKNTLYL |
| | F405L, M428L, | QMNSLRAEDT |
| | N434S mutations and | AVYYCAKVSY |
| | G236 deleted | LSTASSLDYW |
| | | GQGTLVTVSS |
| | | ASTKGPSVFP |
| | | LAPSSKSTSG |
| | | GTAALGCLVK |
| | | DYFPEPVTVS |
| | | WNSGALTSGV |
| | | HTFPAVLQSS |
| | | GLYSLSSVVT |
| | | VPSSSLGTQT |
| | | YICNVNHKPS |
| | | NTKVDKKVEP |
| | | KSCDKTHTCP |
| | | PCPAPPAAGP |
| | | SVFLFPPKPK |
| | | DTLMISRTPE |
| | | VTCVVVDVSH |
| | | EDPEVKFNWY |
| | | VDGVEVHNAK |
| | | TKPREEQYNS |
| | | TYRVVSVLTV |
| | | LHQDWLNGKE |
| | | YKCKVSNKAL |
| | | PAPIEKTISK |
| | | AKGQPREPQV |
| | | YTLPPSRDEL |
| | | TKNQVSLTCL |
| | | VKGFYPSDIA |
| | | VEWESNGQPE |
| | | NNYKTTPPVL |
| | | DSDGSFLLYS |
| | | KLTVDKSRWQ |
| | | QGNVFSCSVL |
| | | HEALHSHYTQ |
| | | KSLSLSPGK |
| SEQ | anti-TNFα shielded | AYRPSETLCG |
| ID | antibody by | GELVDTLQFV |
| NO: | TavoPRECISE-Shield | CGDRGFYFGG |
| 62 | light chain EAC195 | GGSGPLGMLS |
| | based on ADA-L1 | QGGGGSEIVM |
| | variable domain with | TQSPATLSVS |
| | IGF2 B chain | PGERATLSCR |
| | (underlined) and | ASQGIRNYLA |
| | MMP2/9 substrate | WYQQKPGQAP |
| | sequence (bolded) | RLLIYAASTL |
| | | QSGIPARFSG |
| | | SGSGTEFTLT |
| | | ISSLQSEDFA |
| | | VYYCQRYNRA |
| | | PYTFGQGTKV |
| | | EIKRTVAAPS |
| | | VFIFPPSDEQ |
| | | LKSGTASVVC |
| | | LLNNFYPREA |
| | | KVQWKVDNAL |
| | | QSGNSQESVT |
| | | EQDSKDSTYS |
| | | LSSTLTLSKA |
| | | DYEKHKVYAC |
| | | EVTFIQGLSS |
| | | PVTKSFNRGE |
| | | C |
| SEQ | anti-TNFα shielded | GIVEECCFRS |
| ID | antibody by | CDLALLETYC |
| NO: | TavoPRECISE-Shield | AGGGGSLSGR |
| 63 | heavy chain EAC198 | SDNHGGGGSE |
| | based on ADA-H2X | VQLVESGGGL |
| | variable domain with | V |
| | IGF2 A chain | QPGGSLRLSC |
| | (underlined), uPA | AASGFDFADY |
| | substrate sequence | AMHWVRQAPG |
| | (bolded) and IgG1 Fc | KGLEWVSAIT |
| | with E233P, L234A, | WNGGHTDYAD |
| | L235A, F405L, | SVKGRFTISR |
| | M428L, N434S | DNSKNTLYLQ |
| | mutations and G236 | MNSLRAEDTA |
| | deleted | VYYCAKVSYL |
| | | STASSLDYWG |
| | | QGTLVTVSSA |
| | | STKGPSVFPL |
| | | APSSKSTSGG |
| | | TAALGCLVKD |
| | | YFPEPVTVSW |
| | | NSGALTSGVH |
| | | TFPAVLQSSG |
| | | LYSLSSVVTV |
| | | PSSSLGTQTY |
| | | ICNVNHKPSN |
| | | TKVDKKVEPK |
| | | SCDKTHTCPP |
| | | CPAPPAAGPS |
| | | VFLFPPKPKD |
| | | TLMISRTPEV |
| | | TCVVVDVSHE |
| | | DPEVKFNWYV |
| | | DGVEVHNAKT |
| | | KPREEQYNST |
| | | YRVVSVLTVL |
| | | HQDWLNGKEY |
| | | KCKVSNKALP |
| | | APIEKTISKA |
| | | KGQPREPQVY |
| | | TLPPSRDELT |
| | | KNQVSLTCLV |
| | | KGFYPSDIAV |
| | | EWESNGQPEN |
| | | NYKTTPPVLD |
| | | SDGSFLLYSK |
| | | LTVDKSRWQQ |
| | | GNVFSCSVLH |
| | | EALHSHYTQK |
| | | SLSLSPGK |
| SEQ | anti-TNFα shielded | AYRPSETLCG |
| ID | antibody by | GELVDTLQFV |
| NO: | TavoPRECISE-Shield | CGDRGFYFGG |
| 64 | light chain EAC199 | GGSLSGRSDN |
| | based on ADA-L1 | HGGGGSEIVM |
| | variable domain with | TQSPATLSVS |
| | IGF2 B chain | PGERATLSCR |
| | (underlined) and uPA | ASQGIRNYLA |
| | substrate sequence | WYQQKPGQAP |
| | (bolded) | RLLIYAASTL |
| | | QSGIPARFSG |
| | | SGSGTEFTLT |
| | | ISSLQSEDFA |
| | | VYYCQRYNRA |
| | | PYTFGQGTKV |
| | | EIKRTVAAPS |
| | | VFIFPPSDEQ |
| | | LKSGTASVVC |
| | | LLNNFYPREA |
| | | KVQWKVDNAL |
| | | QSGNSQESVT |
| | | EQDSKDSTYS |
| | | LSSTLTLSKA |
| | | DYEKHKVYAC |
| | | EVTHQGLSSP |
| | | VTKSFNRGEC |
| SEQ | anti-TNFα antibody | EVQLVESGGG |
| ID | heavy chain EAC167 | LVQPGGSLRL |
| NO: | based on ADA-H2X | SCAASGFDFA |
| 65 | variable domain with | DYAMHWVRQA |
| | IgG1 Fc with E233P, | PGKGLEWVSA |
| | L234A, L235A, | ITWNGGHTDY |
| | F403L, M428L, | ADSVKGRFTI |
| | N434S mutations and | SRDNSKNTLY |
| | G236 deleted | LQMNSLRAED |
| | | TAVYYCAKVS |
| | | YLSTASSLDY |
| | | WGQGTLVTVS |
| | | SASTKGPSVF |
| | | PLAPSSKSTS |
| | | GGTAALGCLV |
| | | KDYFPEPVTV |
| | | SWNSGALTSG |
| | | VHTFPAVLQS |
| | | SGLYSLSSVV |
| | | TVPSSSLGTQ |
| | | TYICNVNHKP |
| | | SNTKVDKKVE |
| | | PKSCDKTHTC |
| | | PPCPAPPAAG |
| | | PSVFLFPPKP |
| | | KDTLMISRTP |
| | | EVTCVVVDVS |
| | | HEDPEVKFNW |
| | | YVDGVEVHNA |
| | | KTKPREEQYN |
| | | STYRVVSVLT |
| | | VLHQDWLNGK |
| | | EYKCKVSNKA |
| | | LPAPIEKTIS |
| | | KAKGQPREPQ |
| | | VYTLPPSRDE |
| | | LTKNQVSLTC |
| | | LVKGFYPSDI |
| | | AVEWESNGQP |
| | | ENNYKTTPPV |
| | | LDSDGSFLLY |
| | | SkLTVDKSRW |
| | | QQGNVFSCSV |
| | | LHEALHSHYT |
| | | QKSLSLSPGK |
| SEQ | anti-TNFα antibody | EIVMTQSPAT |
| ID | light chain EAC127 | LSVSPGERAT |
| NO: | based on ADA-L1 | LSCRASQGIR |
| 66 | variable domain | NYLAWYQQKP |
| | | GQAPRLLIYA |
| | | ASTLQSGIPA |
| | | RFSGSGSGTE |
| | | FTLTISSLQS |
| | | EDFAVYYCQR |
| | | YNRAPYTFGQ |
| | | GTKVEIKRTV |
| | | AAPSVFIFPP |
| | | SDEQLKSGTA |
| | | SWCLLNNFYP |
| | | REAKVQWKVD |
| | | NALQSGNSQE |
| | | SVTEQDSKDS |
| | | TYSLSSTLTL |
| | | SKADYEKHKV |
| | | YACEVTHQGL |
| | | SSPVTKSFNR |
| | | GEC |
| SEQ | anti-HER2 antibody | EVQLVESGGG |
| ID | heavy chain EAC202 | LVQPGGSLRL |
| NO: | with IgG1 Fc with | SCAASGFNIK |
| 67 | E233P, F405L, | DTYIHWVRQA |
| | M428L, N434S | PGKGLEWVAR |
| | mutations and G236 | IYPTNGYTRY |
| | deleted | ADSVKGRFTI |
| | | SADTSKNTAY |
| | | LQMNSLRAED |
| | | TAVYYCSRWG |
| | | GDGFYAMDYW |
| | | GQGTLVTVSS |
| | | ASTKGPSVFP |
| | | LAPSSKSTSG |
| | | GTAALGCLVK |
| | | DYFPEPVTVS |
| | | WNSGALTSGV |
| | | HTFPAVLQSS |
| | | GLYSLSSVVT |
| | | VPSSSLGTQT |
| | | YICNVNHKPS |
| | | NTKVDKKVEP |
| | | KSCDKTHTCP |
| | | PCPAPPLLGP |
| | | SVFLFPPKPK |
| | | DTLMISRTPE |
| | | VTCVVVDVSH |
| | | EDPEVKFNWY |
| | | VDGVEVHNAK |
| | | TKPREEQYNS |
| | | TYRVVSVLTV |
| | | LHQDWLNGKE |
| | | YKCKVSNKAL |
| | | PAPIEKTISK |
| | | AKGQPREPQV |
| | | YTLPPSRDEL |
| | | TKNQVSLTCL |
| | | VKGFYPSDIA |
| | | VEWESNGQPE |
| | | NNYKTTPPVL |
| | | DSDGSFLLYS |
| | | KLTVDKSRWQ |
| | | QGNVFSCSVL |
| | | HEALHSHYTQ |
| | | KSLSLSPGK |
| SEQ | anti-HER2 antibody | DIQMTQSPSS |
| ID | light chain EAC203 | LSASVGDRVT |
| NO: | | ITCRASQDVN |
| 68 | | TAVAWYQQKP |
| | | GKAPKLUYSA |
| | | SFLYSGVPSR |
| | | FSGSRSGTDF |
| | | TLTISSLQPE |
| | | DFATYYCQQH |
| | | YTTPPTFGQG |
| | | TKVEIKRTVA |
| | | APSVFIFPPS |
| | | DEQLKSGTAS |
| | | WCLLNNFYPR |
| | | EAKVQWKVDN |
| | | ALQSGNSQES |
| | | VTEQDSKDST |
| | | YSLSSTLTLS |
| | | KADYEKHKVY |
| | | ACEVTHQGLS |
| | | SPVTKSFNRG |
| | | EC |
| SEQ | Human IGF2 A chain | GIAEECCFRS |
| ID | with V43A mutation | CDLALLETYC |
| NO: | (bold) | A |
| 69 | | |
| SEQ | Human IGF2 A chain | GILEECCFRS |
| ID | with V43L mutation | CDLALLETYC |
| NO: | (bold) | A |
| 70 | | |
| SEQ | Human IGF2 B chain | AYRPSETLCG |
| ID | with Y27A mutation | GELVDTLQFV |
| NO: | (bold) | CGDRGFAF |
| 71 | | |
| SEQ | Human IGF2 B chain | AYRPSETLCG |
| II) | with Y27L mutation | GELVDTLQFV |
| NO: | (bold) | CGDRGFLF |
| 72 | | |
| SEQ | anti-TNFα shielded | GILEECCFRS |
| ID | antibody heavy chain | CDLALLETYC |
| NO: | EAC271 based on | AGGGGSPLGL |
| 73 | ADA-H2X variable | AGGGGGSEVQ |
| | domain with IGF2 A | LVESGGGLVQ |
| | chain with V43L | PGGSLRLSCA |
| | mutation (underlined), | ASGFDFADYA |
| | MMP2/9 substrate | MHWVRQAPGK |
| | sequence (bolded) and | GLEWVSAITW |
| | IgG1 Fc with E233P, | NGGHTDYADS |
| | L234A, L235A, | VKGRFTISRD |
| | F405L, M428L, | NSKNTLYLQM |
| | N434S mutations and | NSLRAEDTAV |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSWTVPS |
| | | SSLGTQTYIC |
| | | NVNHKPSNTK |
| | | VDKKVEPKSC |
| | | DKTHTCPPCP |
| | | APPAAGPSVF |
| | | LFPPKPKDTL |
| | | MISRTPEVTC |
| | | VVVDVSHEDP |
| | | EVKFNWYVDG |
| | | VEVHNAKTKP |
| | | REEQYNSTYR |
| | | VVSVLTVLHQ |
| | | DWLNGKEYKC |
| | | KVSNKALPAP |
| | | IEKTISKAKG |
| | | QPREPQVYTL |
| | | PPSRDELTKN |
| | | QVSLTCLVKG |
| | | FYPSDIAVEW |
| | | ESNGQPENNY |
| | | KTTPPVLDSD |
| | | GSFLLYSkLT |
| | | VDKSRWQQ |
| | | GNVFSCSVLH |
| | | EALHSHYTQK |
| | | SLSLSPGK |
| SEQ | anti-TNFα shielded | AYRPSETLCG |
| ID | antibody light chain | GELVDTLQFV |
| NO: | EAC272 based on | CGDRGFAFGG |
| 74 | ADA-L1 variable | GGSPLGLAGG |
| | domain with IGF2 B | GGGSEIVMTQ |
| | chain with Y27A | SPATLSVSPG |
| | mutation (underlined) | ERATLSCRAS |
| | and MMP2/9 | QGIRNYLAWY |
| | substrate sequence | QQKPGQAPRL |
| | (bolded) | LIYAASTLQS |
| | | GIPARFSGSG |
| | | SGTEFTLTIS |
| | | SLQSEDFAVY |
| | | YCQRYNRAPY |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-TNFα shielded | GILEECCFRS |
| ID | antibody heavy chain | CDLALLETYC |
| NO: | EAC578 based on | AGGGGSPLGL |
| 75 | ADA-H1 variable | AGGGGGSEVQ |
| | domain with IGF2 A | LVESGGVVVQ |
| | chain with V43L | PGGSLRLSCA |
| | mutation (underlined), | ASGFTFDDYA |
| | MMP2/9 substrate | MHWVRQAPGK |
| | sequence (bolded) and | GLEWVSAITW |
| | IgG1 Fc with E233P, | NSGHIDYADS |
| | L234A, L235A, | VKGRFTISRD |
| | F405L, M428L, | NSKNSLYLQM |
| | N434S mutations and | NSLRTEDTAL |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVWDVSHEDP |
| | | EVKFNWYVDG |
| | | VEVHNAKTKP |
| | | REEQYNSTYR |
| | | VVSVLTVLHQ |
| | | DWLNGKEYKC |
| | | KVSNKALPAP |
| | | IEKTISKAKG |
| | | QPREPQVYTL |
| | | PPSRDELTKN |
| | | QVSLTCLVKG |
| | | FYPSDIAVEW |
| | | ESNGQPENNY |
| | | KTTPPVLDSD |
| | | GSFLLYSkL |
| | | TVDKSRWQQ |
| | | GNVFSCSVLH |
| | | EALHSHYTQK |
| | | SLSLSPGK |
| SEQ | anti-TNFα shielded | GILEECCFRS |
| ID | antibody heavy chain | CDLALLETYC |
| NO: | EAC579 based on | AGGGGSPLGL |
| 76 | ADA-H1X variable | AGGGGGSEVQ |
| | domain with IGF2 A | LVESGGVVVQ |
| | chain with V43L | PGGSLRLSCA |
| | mutation (underlined), | ASGFDFADYA |
| | MMP2/9 substrate | MHWVRQAPGK |
| | sequence (bolded) and | GLEWVSAITW |
| | IgG1 Fc with E233P, | NGGHTDYADS |
| | L234A, L235A, | VKGRFTISRD |
| | F405L, M428L, | NSKNSLYLQM |
| | N434S mutations and | NSLRTEDTAL |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQ |
| | | GNVFSCSVLH |
| | | EALHSHYTQK |
| | | SLSLSPGK |
| SEQ | anti-TNFα shielded | GILEECCFRS |
| ID | antibody heavy chain | CDLALLETYC |
| NO: | EAC580 based on | AGGGGSPLGL |
| 77 | ADA-H2 variable | AGGGGGSEVQ |
| | domain with IGF2 A | LVESGGGLVQ |
| | chain with V43L | PGGSLRLSCA |
| | mutation (underlined), | ASGFTFDDYA |
| | MMP2/9 substrate | MHWVRQAPGK |
| | sequence (bolded) and | GLEWVSAITW |
| | IgG1 Fc with E233P, | NSGHIDYADS |
| | L234A, L235A, | VKGRFTISRD |
| | F405L, M428L, | NSKNTLYLQM |
| | N434S mutations and | NSLRAEDTAV |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGA |
| | | LTSGVHTFPA |
| | | VLQSSGLYSL |
| | | SSVVTVPSSS |
| | | LGTQTYICNV |
| | | NHKPSNTKVD |
| | | KKVEPKSCDK |
| | | THTCPPCPAP |
| | | PAAGPSVFLF |
| | | PPKPKDTLMI |
| | | SRTPEVTCVW |
| | | DVSHEDPEVK |
| | | FNWYVDGVEV |
| | | HNAKTKPREE |
| | | QYNSTYRVVS |
| | | VLTVLHQDWL |
| | | NGKEYKCKVS |
| | | NKALPAPIEK |
| | | TISKAKGQPR |
| | | EPQVYTLPPS |
| | | RDELTKNQVS |
| | | LTCLVKGFYP |
| | | SDIAVEWESN |
| | | GQPENNYKTT |
| | | PPVLDSDGSF |
| | | LLYSkLTVDK |
| | | SRWQQGNVFS |
| | | CSVLHEALHS |
| | | HYTQKSLSLS |
| | | PGK |
| SEQ | anti-TNFa shielded | GILEECCFRS |
| ID | antibody heavy chain | CDLALLETYC |
| NO: | EAC584 based on | AGGGGSPLGL |
| 78 | ADA-H4X variable | AGGGGGSQVQ |
| | domain with IGF2 A | LVESGGGVVQ |
| | chain with V43L | PGGSLRLSCA |
| | mutation (underlined), | ASGFDFADYA |
| | MMP2/9 substrate | MHWVRQAPGK |
| | sequence (bolded) and | GLEWVSAITW |
| | IgG1 Fc with E233P, | NGGHTDYADS |
| | L234A, L235A, | VKGRFTISRD |
| | F405L, M428L, | NSKNTLYLQM |
| | N434S mutations and | NSLRAEDTAV |
| | G236 deleted | YYCAKVSYLS |
| | | TASSLDYWGQ |
| | | GTLVTVSSAS |
| | | TKGPSVFPLA |
| | | PSSKSTSGGT |
| | | AALGCLVKDY |
| | | FPEPVTVSWN |
| | | SGALTSGVHT |
| | | FPAVLQSSGL |
| | | YSLSSVVTVP |
| | | SSSLGTQTYI |
| | | CNVNHKPSNT |
| | | KVDKKVEPKS |
| | | CDKTHTCPPC |
| | | PAPPAAGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQG |
| | | NVFSCSVLHE |
| | | ALHSHYTQKS |
| | | LSLSPGK |
| SEQ | anti-TNFa shielded | AYRPSETLCG |
| ID | antibody light chain | GELVDTLQFV |
| NO: | EAC585 based on | CGDRGFAFGG |
| 79 | ADA-L2 variable | GGSPLGLAGG |
| | domain with IGF2 B | GGGSDIVMTQ |
| | chain with Y27A | SPDSLAVSLG |
| | mutation (underlined) | ERATINCRAS |
| | and MMP2/9 | QGIRNYLAWY |
| | substrate sequence | QQKPGQAPKL |
| | (bolded) | LIYAASTLQS |
| | | GVPDRFSGSG |
| | | SGTDFTLTIS |
| | | SLQAEDVAVY |
| | | YCQRYNRAPY |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | shielded anti-HER2 | GILEECCFRS |
| ID | antibody heavy chain | CDLALLETYC |
| NO: | EAC293 with 1GF2 A | AGGGGSPLGL |
| 80 | chain with V43L | AGGGGGSEVO |
| | mutation (underlined), | LVESGGGLVQ |
| | MMP2/9 substrate | PGGSLRLSCA |
| | sequence (bolded) and | ASGFNIKDTY |
| | IgG1 Fc with F405L, | IHWVRQAPGK |
| | M428L, N434S | GLEWVARIYP |
| | mutations | TNGYTRYADS |
| | | VKGRFTISAD |
| | | TSKNTAYLQM |
| | | NSLRAEDTAV |
| | | YYCSRWGGDG |
| | | FYAMDYWGQG |
| | | TLVTVSSAST |
| | | KGPSVFPLAP |
| | | SSKSTSGGTA |
| | | ALGCLVKDYF |
| | | PEPVTVSWNS |
| | | GALTSGVHTF |
| | | PAVLQSSGLY |
| | | SLSSVVTVPS |
| | | SSLGTQTYIC |
| | | NVNHKPSNTK |
| | | VDKKVEPKSC |
| | | DKTHTCPPCP |
| | | APELLGGPSV |
| | | FLFPPKPKDT |
| | | LMISRTPEVT |
| | | CVVVDVSHED |
| | | PEVKFNWYVD |
| | | GVEVHNAKTK |
| | | PREEQYNSTY |
| | | RVVSVLTVLH |
| | | QDWLNGKEYK |
| | | CKVSNKALPA |
| | | PIEKTISKAK |
| | | GQPREPQVYT |
| | | LPPSRDELTK |
| | | NQVSLTCLVK |
| | | GFYPSDIAVE |
| | | WESNGQPENN |
| | | YKTTPPVLDS |
| | | DGSFLLYSkL |
| | | TVDKSRWQQG |
| | | NVFSCSVLHE |
| | | ALHSHYTQKS |
| | | LSLSPGK |
| SEQ | shielded anti-HER2 | AYRPSETLCG |
| ID | antibody light chain | GELVDTLQFV |
| NO: | EAC294 with IGF2 B | CGDRGFAFGG |
| 81 | chain with Y27A | GGSPLGLAGG |
| | mutation (underlined) | GGGSDIQMTQ |
| | and MMP2/9 | SPSSLSASVG |
| | substrate sequence | DRVTITCRAS |
| | (bolded) | QDVNTAVAWY |
| | | QQKPGKAPKL |
| | | LIYSASFLYS |
| | | GVPSRFSGSR |
| | | SGTDFTLTIS |
| | | SLQPEDFATY |
| | | YCQQHYTTPP |
| | | TFGQGTKVEI |
| | | KRTVAAPSVF |
| | | IFPPSDEQLK |
| | | SGTASVVCLL |
| | | NNFYPREAKV |
| | | QWKVDNALQS |
| | | GNSQESVTEQ |
| | | DSKDSTYSLS |
| | | STLTLSKADY |
| | | EKHKVYACEV |
| | | THQGLSSPVT |
| | | KSFNRGEC |
| SEQ | anti-HER2 antibody | EVQLVESGGG |
| ID | heavy chain EAC289 | LVQPGGSLRL |
| NO: | with IgG1 Fc with | SCAASGFNIK |
| 82 | F405L, M428L, | DTYIHWVRQA |
| | N434S mutations | PGKGLEWVAR |
| | | IYPTNGYTRY |
| | | ADSVKGRFTI |
| | | SADTSKNTAY |
| | | LQMNSLRAED |
| | | TAVYYCSRWG |
| | | GDGFYAMDYW |
| | | GQGTLVTVSS |
| | | ASTKGPSVFP |
| | | LAPSSKSTSG |
| | | GTAALGCLVK |
| | | DYFPEPVTVS |
| | | WNSGALTSGV |
| | | HTFPAVLQSS |
| | | GLYSLSSVVT |
| | | VPSSSLGTQT |
| | | YICNVNHKPS |
| | | NTKVDKKVEP |
| | | KSCDKTHTCP |
| | | PCPAPELLGG |
| | | PSVFLFPPKP |
| | | KDTLMISRTP |
| | | EVTCVVVDVS |
| | | HEDPEVKFNW |
| | | YVDGVEVHNA |
| | | KTKPREEQYN |
| | | STYRVVSVLT |
| | | VLHQDWLNGK |
| | | EYKCKVSNKA |
| | | LPAPIEKTIS |
| | | KAKGQPREPQ |
| | | VYTLPPSRDE |
| | | LTKNQVSLTC |
| | | LVKGFYPSDI |
| | | AVEWESNGQP |
| | | ENNYKTTPPV |
| | | LDSDGSFLLY |
| | | SkLTVDKSRW |
| | | QQGNVFSCSV |
| | | LHEALHSHYT |
| | | QKSLSLSPGK |

### REFERENCES

Agunbiade, T. A., R. Y. Zaghlol and A. Barac (2019). “Heart Failure in Relation to Tumor-Targeted Therapies and Immunotherapies.” Methodist Debakey Cardiovasc J 15(4): 250-257.
Autio, K. A., V. Boni, R. W. Humphrey and A. Naing (2020). “Probody Therapeutics: An Emerging Class of Therapies Designed to Enhance On-Target Effects with Reduced Off-Tumor Toxicity for Use in Immuno-Oncology.” Clin Cancer Res 26(5): 984-989.
Banys-Paluchowski, M., I. Witzel, B. Aktas, P. A. Fasching, A. Hartkopf, W. Janni, S. Kasimir-Bauer, K. Pantel, G. Schon, B. Rack, S. Riethdorf, E. F. Solomayer, T. Fehm and V. Muller (2019). “The prognostic relevance of urokinase-type plasminogen activator (uPA) in the blood of patients with metastatic breast cancer.” Sci Rep 9(1): 2318.
Chen, I. J., C. H. Chuang, Y. C. Hsieh, Y. C. Lu, W. W. Lin, C. C. Huang, T. C. Cheng, Y. A. Cheng, K. W. Cheng, Y. T. Wang, F. M. Chen, T. L. Cheng and S. C. Tzou (2017). “Selective antibody activation through protease-activated pro-antibodies that mask binding sites with inhibitory domains.” Sci Rep 7(1): 11587.
Chen, X., J. L. Zaro and W. C. Shen (2013). “Fusion protein linkers: property, design and functionality.” Adv Drug Deliv Rev 65(10): 1357-1369.
Dall'Acqua, W. F., P. A. Kiener and H. Wu (2006). “Properties of human IgG₁s engineered for enhanced binding to the neonatal Fc receptor (FcRn).” J Biol Chem 281(33): 23514-23524.
Di Giacomo, A. M., M. Biagioli and M. Maio (2010). “The emerging toxicity profiles of anti-CTLA-4 antibodies across clinical indications.” Semin Oncol 37(5): 499-507.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343(6176): 1260-1263.
Donaldson, J. M., C. Kari, R. C. Fragoso, U. Rodeck and J. C. Williams (2009). “Design and development of masked therapeutic antibodies to limit off-target effects: application to anti-EGFR antibodies.” Cancer Biol Ther 8(22): 2147-2152.
Fernandez-Ruiz, M. and J. M. Aguado (2018). “Risk of infection associated with anti-TNF-alpha therapy.” Expert Rev Anti Infect Ther 16(12): 939-956.
Fiorentino, D., V. Ho, M. G. Lebwohl, L. Leite, L. Hopkins, C. Galindo, K. Goyal, W. Langholff, S. Fakharzadeh, B. Srivastava and R. G. Langley (2017). “Risk of malignancy with systemic psoriasis treatment in the Psoriasis Longitudinal Assessment Registry.” J Am Acad Dermatol 77(5): 845-854 e845.
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody F_{c} heterodimer formation through electrostatic steering effects: applications to bispecific molecules and monovalent IgG.” J Biol Chem 285(25): 19637-19646.
Haluska, P., M. Menefee, E. R. Plimack, J. Rosenberg, D. Northfelt, T. LaVallee, L. Shi, X. Q. Yu, P. Burke, J. Huang, J. Viner, J. McDevitt and P. LoRusso (2014). “Phase I dose-escalation study of MEDI-573, a bispecific, antiligand monoclonal antibody against IGFI and IGFII, in patients with advanced solid tumors.” Clin Cancer Res 20(18): 4747-4757.
Hinton, P. R., J. M. Xiong, M. G. Johlfs, M. T. Tang, S. Keller and N. Tsurushita (2006). “An engineered human IgG1 antibody with longer serum half-life.” J Immunol 176(1): 346-356.
Kavanaugh, W. M. (2020). “Antibody prodrug for cancer.” Expert Opin Biol Ther 20(2): 163-171.
Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brezski (2013). “Engineered protease-resistant antibodies with selectable cell-killing functions.” J Biol Chem 288(43): 30843-30854.
Klement, R. J. and M. K. Fink (2016). “Dietary and pharmacological modification of the insulin/IGF-1 system: exploiting the full repertoire against cancer.” Oncogenesis 5: e193.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2013). “Efficient generation of stable bispecific IgG1 by controlled Fab-arm exchange.” Proc Natl Acad Sci USA 110(13): 5145-5150.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG1 by controlled Fab-arm exchange.” Proc Natl Acad Sci USA 110(13): 5145-5150.
Lin, W. W., Y. C. Lu, C. H. Chuang and T. L. Cheng (2020). “Ab locks for improving the selectivity and safety of antibody drugs.” J Biomed Sci 27(1): 76.
Livingstone, C. (2013). “IGF2 and cancer.” Endocr Relat Cancer 20(6): R321-339.
Lu, Y. C., C. H. Chuang, K. H. Chuang, I. J. Chen, B. C. Huang, W. H. Lee, H. E. Wang, J. J. Li, Y. A. Cheng, K. W. Cheng, J. Y. Wang, Y. C. Hsieh, W. W. Lin and T. L. Cheng (2019). “Specific activation of pro-Infliximab enhances selectivity and safety of rheumatoid arthritis therapy.” PLoS Biol 17(6): e3000286.
Metz, S., C. Panke, A. K. Haas, J. Schanzer, W. Lau, R. Croasdale, E. Hoffmann, B. Schneider, J. Auer, C. Gassner, B. Bossenmaier, P. Umana, C. Sustmann and U. Brinkmann (2012). “Bispecific antibody derivatives with restricted binding functionalities that are activated by proteolytic processing.” Protein Eng Des Sel 25(10): 571-580.
Moore, A. R., S. Allden, T. Bourne, M. C. Denis, K. Kranidioti, R. Okoye, Y. Sotsios, Z. Stencel, A. Vugler, G. Watt and S. Shaw (2014). “Collagen II antibody-induced arthritis in Tg1278TNFko mice: optimization of a novel model to assess treatments targeting human TNFalpha in rheumatoid arthritis.” J Transl Med 12: 285.
Mullard, A. (2021). “FDA approves 100th monoclonal antibody product.” Nat Rev Drug Discov 20(7): 491-495.
Nair, V. B., C. S. Samuel, F. Separovic, M. A. Hossain and J. D. Wade (2012). “Human relaxin-2: historical perspectives and role in cancer biology.” Amino Acids 43(3): 1131-1140.
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18(12): 1759-1769.
Ridgway, J. B., L. G. Presta and P. Carter (1996). “‘Knobs-into-holes’ engineering of antibody CH3 domains for heavy chain heterodimerization.” Protein Eng 9(7): 617-621.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Spiers, L., N. Coupe and M. Payne (2019). “Toxicities associated with checkpoint inhibitors—an overview.” Rheumatology (Oxford) 58(Suppl 7): vii7-vii16.
Touyz, R. M., S. M. S. Herrmann and J. Herrmann (2018). “Vascular toxicities with VEGF inhibitor therapies-focus on hypertension and arterial thrombotic events.” J Am Soc Hypertens 12(6): 409-425.
Trang, V. H., X. Zhang, R. C. Yumul, W. Zeng, I. J. Stone, S. W. Wo, M. M. Dominguez, J. H. Cochran, J. K. Simmons, M. C. Ryan, R. P. Lyon, P. D. Senter and M. R. Levengood (2019). “A coiled-coil masking domain for selective activation of therapeutic antibodies.” Nat Biotechnol 37(7): 761-765.
Vafa, O., G. L. Gilliland, R. J. Brezski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65(1): 114-126.
Worn, A. and A. Pluckthun (2001). “Stability engineering of antibody single-chain Fv fragments.” J Mol Biol 305(5): 989-1010.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200(1): 16-26.
Yu, H., J. Mistry, M. J. Nicar, M. J. Khosravi, A. Diamandis, J. van Doorn and A. Juul (1999). “Insulin-like growth factors (IGF-I, free IGF-I and IGF-II) and insulin-like growth factor binding proteins (IGFBP-2, IGFBP-3, IGFBP-6, and ALS) in blood circulation.” J Clin Lab Anal 13(4): 166-172.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel F_{c} mutations to promote antibody multimerization.” MAbs 9: 1129-1142.
Zhang, W., Q. Huang, W. Xiao, Y. Zhao, J. Pi, H. Xu, H. Zhao, J. Xu, C. E. Evans and H. Jin (2020). “Advances in Anti-Tumor Treatments Targeting the CD47/SIRPalpha Axis.” Front Immunol 11: 18.

We claim:

1. A shielded biologic comprising:
a) a heavy chain polypeptide comprising, from N-terminus to C-terminus, a first masking domain unit, a first protease-cleavable linker, and an antibody heavy chain or an antigen-binding fragment thereof; and
b) a light chain polypeptide comprising, from N-terminus to C-terminus, a second masking domain unit, a second protease-cleavable linker, and an antibody light chain or an antigen-binding fragment thereof, wherein the first masking domain unit and the second masking domain unit form a masking domain.

2. The shielded biologic of claim 1,
wherein the first masking domain unit comprises a sequence based on the A chain or B chain of Insulin Growth Factor 2 (IGF2), and the second masking domain unit comprises a sequence based on the B chain or A chain of IGF2.

3. The shielded biologic of claim 1, wherein the masking domain is capable of shielding the antigen binding capability of the biologics in normal tissues, and wherein in target disease tissues, one or both of the masking domain units are cleaved off by disease site-specific proteases and the shielded biologic is converted to an active antibody.

4. The shielded biologic of claim 1, wherein the shielded biologic comprises a heavy chain polypeptide and a light chain polypeptide selected from the heavy chain and light chain sequences listed in Tables 2, 3, 4, and 5.

5. The shielded biologic of claim 1, wherein the shielded biologic comprises a combination of a heavy chain and a light chain with an amino acid sequence having at least 80% identity to the respective heavy chain and light chain amino acid sequences chosen from SEQ ID NO: 1 to 16, 41 to 52, 55 to 64, and 73 to 81.

6. The shielded biologic of claim 1, wherein the first and second masking domain units comprise sequences derived from human IGF2 pre-prohormone comprising an amino acid sequence having at least 80% identity to the amino acid sequence set forth as SEQ ID NO: 17.

7. The shielded biologic of claim 1, wherein the first or second masking domain unit comprises a human IGF2 A chain sequence comprising an amino acid sequence having at least 80% amino acid identity to the acid sequence set forth as SEQ ID NO: 18, and the first or second masking domain unit comprises a human IGF2 B chain sequence comprising an amino acid sequence having at least 80% amino acid identity to the sequence set forth as SEQ ID NO: 19, and wherein the A chain and B chain of IGF2 forms a heterodimeric complex through two interchain disulfide bonds.

8. The shielded biologic of claim 1, wherein the first or second masking domain unit comprises human IGF2 A chain or B chain with mutations chosen from SEQ ID NO: 69-72, wherein the mutations disrupt IGF2 binding to its receptor.

9. The shielded biologic of claim 1, wherein the first or second masking domain unit comprises human IGF2 A chain with a V43L mutation set forth as SEQ ID NO: 70 or human IGF2 B chain with a Y27A mutation set forth as SEQ ID NO: 71, wherein the mutations disrupt IGF2 binding to its receptor.

10. The shielded biologic of claim 1, wherein the first and second masking domain units comprise sequences derived from pre-prohormone sequences of a member of Insulin-Relaxin superfamily, comprising amino acid sequences having at least 80% identity to the amino acid sequence chosen from SEQ ID NO: 21 to 29.

11. The shielded biologic of claim 1, wherein the first and second protease-cleavable linkers comprise a protease substrate sequence for a protease enriched in target disease site chosen from matrix metalloprotease, Cathepsin, urokinase plasminogen activator (uPA), disintegrin and metalloproteinase, wherein the first and second protease-cleavable linkers may comprise the same or different protease substrate sequences.

12. The shielded biologic of claim 1, wherein the first and/or second protease-cleavable linkers comprise a substrate sequence having at least 80% identity to the amino acid sequence chosen from SEQ ID NO: 30 to 40.

13. The shielded biologic of claim 1, wherein the shielded biologic is against one or more therapeutic targets chosen from, but are not limited to, TNFα, IL-1β, HER2, VEGF, EGFR, cMET, Nectin-4, CTLA-4, CD3ε, a4-integrin, CD20, CD11a, CD52, RANK-L, PD-1, PD-L1, CD47, CD24, CD166, and CD71.

14. The shielded biologic of claim 1, wherein the shielded biologic is a monoclonal antibody, bispecific antibody, or multi-specific antibody with IgG, IgA, IgD, IgM subtypes.

15. The shielded biologic of claim 1, wherein the shielded biologic comprises an antigen-binding fragment, or an antigen-binding fragment in antibody drug conjugate format, or an antigen-binding fragment in a chimeric antigen receptor format, or an antigen binding fragment on a non-antibody scaffold.

16. The shielded biologic of claim 1, wherein the heavy chain polypeptide has one or more F_{c} mutations that extend the half-life of the shielded biologic when compared to the native shielded biologic without the one or mutations.

17. The shielded biologic of claim 1, wherein the heavy chain polypeptide has one or more sets of mutations selected from M252Y/S254T/T256E, M428L/N434S, T250Q/M428L, N434A and T307A/E380A/N434A when compared to the native shielded biologic without the mutations, according to the EU Index residue numbering.

18. The shielded biologics of claim 1, wherein the heavy chain polypeptide has one or more F_{c} mutations that enhance the resistance of the shielded biologics to proteolytic degradation by a protease that cleaves the native shielded biologic without the one or more mutations between or at residues 222-237, according to the EU Index residue numbering.

19. The shielded biologics of claim 1, wherein the heavy chain polypeptide comprises E233P/L234A/L235A Fc mutations with G236 deleted when compared to the native shielded biologic without the mutations, residue numbering according to the EU Index residue numbering.

20. The shielded biologic of claim 1, wherein the heavy chain polypeptide has one or more F_{c} mutations that reduce or eliminate the effector functions of engineered shielded biologic compared to the native shielded biologic without the one or more mutations.

21. The shielded biologic of claim 1, wherein the heavy chain polypeptide has L234A, L235A, M428L, and N434S F_{c} mutations that extend the half-life and reduce the effector functions of the engineered shielded biologic, residue numbering according to the EU Index, compared to the shielded biologic.

22. The shielded biologic of claim 1, wherein the heavy chain polypeptide has E233P, L234A, L235A, M428L, and N434S F_{c} mutations with G236 deleted that extend the half-life, reduce the effector functions, and enhance the resistance of the shielded biologic to proteolytic degradation by a protease, residue numbering according to the EU Index, compared to the native shielded biologic.

23. The shielded biologic of claim 1, wherein the heavy chain polypeptide has one or more F_{c} mutations chosen from an F405L mutation and a K409R mutation, wherein the one or more mutations can facilitate heavy chain heterodimerization when compared to the native shielded biologic without the one or more mutations, residue numbering according to the EU Index.

24. An isolated polynucleotide encoding the shielded biologic of claim 1.

25. A vector comprising the polynucleotide of claim 24.

26. The vector of claim 25, which is an expression vector.

27. A host cell comprising the vector of claim 25.

28. A pharmaceutical composition comprising the shielded biologic of claim 1 and a pharmaceutical carrier.

29. A method of producing the shielded biologic of claim 1, comprising culturing a host cell comprising a vector comprising a polynucleotide encoding the shielded biologic of claim 1 in conditions wherein the shielded biologic is expressed, and isolating the shielded biologic.

30. A method of measuring the half-life of the shielded biologic of claim 1.

31. A method of measuring the resistance to proteolytic degradation of the shielded biologic of claim 1.

32. A method of measuring the effector functions of the shielded biologic of claim 1.

33. A method of cleaving the first and/or the second masking domain unit off the shielded biologic of claim 1, by one or more proteases, and converting the shielded biologic into an active antibody.

34. A method of detecting the cleavage of the first and/or the second masking domain unit from the shielded biologic of claim 1, by one or more proteases.

35. A method of measuring the antigen-binding capability of the shielded biologic of claim 1 and the antibody after the first and/or the second masking domain unit is cleaved off by one or more proteases.

36. A method of measuring the functional activities of the shielded biologic of claim 1 and the antibody after the first and/or the second masking domain unit is cleaved off by one or more proteases by in vitro assays.

37. A method of measuring the in vivo efficacy of the shielded biologic of claim 1 and the antibody after the first and/or the second masking domain unit is cleaved off by one or more proteases by one or more animal drug potency studies.

38. A method of measuring the safety profile of the shielded biologic of claim 1 and the antibody after the first and/or the second masking domain unit is cleaved off by one or more proteases by one or more animal toxicity models.

39. A method for treating an auto-immune and/or inflammatory disease in a subject in need thereof, comprising administering to the subject an effective amount of the shielded biologic of claim 1, wherein the disease is chosen from rheumatoid arthritis, systemic lupus erythematosus, osteoarthritis, ankylosing spondylitis, Behcet's Disease, gout, psoriatic arthritis, multiple sclerosis, Crohn's colitis, and inflammatory bowel disease.

40. A method for treating a condition in a subject in need thereof, comprising administering to the subject an effective amount of the shielded biologic of claim 1, wherein the condition is chosen from Type II diabetes mellitus, Parkinson's disease, age-related macular degeneration, polyneuropathy, sensory peripheral neuropathy, proliferative diabetic retinopathy, diabetic neuropathy, decubitus ulcer, fulminant Type 1 diabetes, retinal vasculitis, non-infectious posterior uveitis, and alcoholic neuropathy.

41. A method for treating cancer in a subject in need thereof, comprising administering to the subject an effective amount of the shielded biologic of claim 1, wherein the cancer is chosen from multiple myeloma, non-small cell lung cancer, acute myeloid leukemia, female breast cancer, pancreatic cancer, colorectal cancer and peritoneum cancer.

42. The method of claim 39, wherein said administering is subcutaneous.

43. The method of claim 39, wherein said administering is intravenous.

44. The method of claim 39, wherein said administering is intramuscular.

45. The method of claim 39, wherein said administering is oral or rectal.

46. The method of claim 39, wherein said administering is systemic.

47. The method of claim 39, wherein said administering is local.


SEQUENCE LISTING (102 sequences)

SEQ ID NO: 1 - protein, 487 aa, Artificial Sequence
  Description of Artificial Sequence Synthetic polypeptide
GIVEECCFRSCDLALLETYCAGGGGSPLGLAGGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 2 - protein, 258 aa, Artificial Sequence
  Description of Artificial Sequence Synthetic polypeptide
AYRPSETLCGGELVDTLQFVCGDRGFYFGGGGSPLGLAGGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 491 aa, Artificial Sequence
  Description of Artificial Sequence Synthetic polypeptide
GIVEECCFRSCDLALLETYCAGGGGSRPKPVEVWRKGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 4 - protein, 262 aa, Artificial Sequence
  Description of Artificial Sequence Synthetic polypeptide
AYRPSETLCGGELVDTLQFVCGDRGFYFGGGGSRPKPVEVWRKGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 487 aa, Artificial Sequence
  Description of Artificial Sequence Synthetic polypeptide
GIVEECCFRSCDLALLETYCAGGGGSPLGLAGGGGGSEVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 6 - protein, 263 aa, Artificial Sequence
  Description of Artificial Sequence Synthetic polypeptide
AYRPSETLCGGELVDTLQFVCGDRGFYFGGGGSPLGLAGGGGGSDIVMTQTPLSLPVTPGEPASISCKSSQSLLNSRTRKNYLAWYLQKPGQSPQLLIYWASTRESGVPDRFSGSGSGTDFTLKISRVEAEDVGVYYCKQTYNFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 491 aa, Artificial Sequence
  Description of Artificial Sequence Synthetic polypeptide
GIVEECCFRSCDLALLETYCAGGGGSRPKPVEVWRKGGGGSEVQLVESGGGLVQPGGSLRLSCAASGFTFSSFGMHWVRQAPGKGLEWVAYISIGSYTVHYADSVKGRFTISRDNAKNSLYLQMNSLRDEDTAVYYCVRDDYDVTDYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 8 - protein, 267 aa, Artificial Sequence
  Description of Artificial Sequence Synthetic polypeptide
AYRPSETLCGGELVDTLQFVCGDRGFYFGGGGSRPKPVEVWRKGGGGSDIVMTQTPLSLPVTPGEPASISCKSSQSLLNSRTRKNYLAWYLQKPGQSPQLLIYWASTRESGVPDRFSGSGSGTDFTLKISRVEAEDVGVYYCKQTYNFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 486 aa, Artificial Sequence
  Description of Artificial Sequence Synthetic polypeptide
GIVEECCFRSCDLALLETYCAGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 10 - protein, 258 aa, Artificial Sequence
  Description of Artificial Sequence Synthetic polypeptide
AYRPSETLCGGELVDTLQFVCGDRGFYFGGGGSPLGLAGGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 488 aa, Artificial Sequence
  Description of Artificial Sequence Synthetic polypeptide
GIVEECCFRSCDLALLETYCAGGGGSLSGRSDNHGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 12 - protein, 260 aa, Artificial Sequence
  Description of Artificial Sequence Synthetic polypeptide
AYRPSETLCGGELVDTLQFVCGDRGFYFGGGGSLSGRSDNHGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 489 aa, Artificial Sequence
  Description of Artificial Sequence Synthetic polypeptide
GIVEECCFRSCDLALLETYCAGGGGSPLGLAGGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 14 - protein, 258 aa, Artificial Sequence
  Description of Artificial Sequence Synthetic polypeptide
AYRPSETLCGGELVDTLQFVCGDRGFYFGGGGSPLGLAGGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 491 aa, Artificial Sequence
  Description of Artificial Sequence Synthetic polypeptide
GIVEECCFRSCDLALLETYCAGGGGSLSGRSDNHGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 16 - protein, 260 aa, Artificial Sequence
  Description of Artificial Sequence Synthetic polypeptide
AYRPSETLCGGELVDTLQFVCGDRGFYFGGGGSLSGRSDNHGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 18 - protein, 21 aa, Homo sapiens
GIVEECCFRSCDLALLETYCA

SEQ ID NO: 19 - protein, 28 aa, Homo sapiens
AYRPSETLCGGELVDTLQFVCGDRGFYF

SEQ ID NO: 20 - protein, 12 aa, Homo sapiens
SRPASRVSRRSR

SEQ ID NO: 21 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 22 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 23 - protein, 185 aa, Homo sapiens
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 24 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 25 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 26 - protein, 131 aa, Homo sapiens
MDPRLPAWALVLLGPALVFALGPAPTPEMREKLCGHHFVRALVRVCGGPRWSTEARRPATGGDRELLQWLERRHLLHGLVADSNLTLGPGLQPLPQTSHHHRHHRAAATNPARYCCLSGCTQQDLLTLCPY

SEQ ID NO: 27 - protein, 139 aa, Homo sapiens
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

SEQ ID NO: 28 - protein, 135 aa, Homo sapiens
MKGSIFTLFLFSVLFAISEVRSKESVRLCGLEYIRTVIYICASSRWRRHQEGIPQAQQAETGNSFQLPHKREFSEENPAQNLPKVDASGEDRLWGGQMPTEELWKSKKHSVMSRQDLQTLCCTDGCSMTDLSALC

SEQ ID NO: 29 - protein, 213 aa, Homo sapiens
MPRLLRLSLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAKFQKKRRNKIKTLSNLFWGHHPQRKRRGYSEKCCLTGCTKEELSIACLPYIDFKRLKEKRSSLVTKIY

SEQ ID NO: 30 - protein, 16 aa, Artificial Sequence
  Description of Artificial Sequence Synthetic peptide
GGGGSGPLGVRGGGGS

SEQ ID NO: 31 - protein, 18 aa, Artificial Sequence
  Description of Artificial Sequence Synthetic peptide
GGGGSLSGRSDNHGGGGS

SEQ ID NO: 32 - protein, 20 aa, Artificial Sequence
  Description of Artificial Sequence Synthetic peptide
GGGGSRPKPVEVWRKGGGGS

SEQ ID NO: 33 - protein, 6 aa, Artificial Sequence
  Description of Artificial Sequence Synthetic peptide
GPLGVR

SEQ ID NO: 34 - protein, 6 aa, Artificial Sequence
  Description of Artificial Sequence Synthetic peptide
PLGLAR

SEQ ID NO: 35 - protein, 6 aa, Artificial Sequence
  Description of Artificial Sequence Synthetic peptide
PLGLAG

SEQ ID NO: 36 - protein, 8 aa, Artificial Sequence
  Description of Artificial Sequence Synthetic peptide
IPVSLRSG

SEQ ID NO: 37 - protein, 8 aa, Artificial Sequence
  Description of Artificial Sequence Synthetic peptide
GPLGMLSQ

SEQ ID NO: 38 - protein, 10 aa, Artificial Sequence
  Description of Artificial Sequence Synthetic peptide
RPKPVEVWRK

SEQ ID NO: 39 - protein, 8 aa, Artificial Sequence
  Description of Artificial Sequence Synthetic peptide
LSGRSDNH

SEQ ID NO: 40 - protein, 8 aa, Artificial Sequence
  Description of Artificial Sequence Synthetic peptide
TGRGPSWV

SEQ ID NO: 41 - protein, 488 aa, Artificial Sequence
  Description of Artificial Sequence Synthetic polypeptide
GILEECCFRSCDLALLETYCAGGGGSPLGLAGGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 258 aa, Artificial Sequence
  Description of Artificial Sequence Synthetic polypeptide
AYRPSETLCGGELVDTLQFVCGDRGFAFGGGGSPLGLAGGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 492 aa, Artificial Sequence
  Description of Artificial Sequence Synthetic polypeptide
GILEECCFRSCDLALLETYCAGGGGSRPKPVEVWRKGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 262 aa, Artificial Sequence
  Description of Artificial Sequence Synthetic polypeptide
AYRPSETLCGGELVDTLQFVCGDRGFAFGGGGSRPKPVEVWRKGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 485 aa, Artificial Sequence
  Description of Artificial Sequence Synthetic polypeptide
GILEECCFRSCDLALLETYCAGGGGSPLGLAGGGGGSQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 258 aa, Artificial Sequence
  Description of Artificial Sequence Synthetic polypeptide
AYRPSETLCGGELVDTLQFVCGDRGFAFGGGGSPLGLAGGGGGSEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 486 aa, Artificial Sequence
  Description of Artificial Sequence Synthetic polypeptide
GILEECCFRSCDLALLETYCAGGGGSPLGLAGGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 258 aa, Artificial Sequence
  Description of Artificial Sequence Synthetic polypeptide
AYRPSETLCGGELVDTLQFVCGDRGFAFGGGGSPLGLAGGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 487 aa, Artificial Sequence
  Description of Artificial Sequence Synthetic polypeptide
GILEECCFRSCDLALLETYCAGGGGSLSGRSDNHGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 261 aa, Artificial Sequence
  Description of Artificial Sequence Synthetic polypeptide
AYRPSETLCGGELVDTLQFVCGDRGFAFGGGGSLSGRSDNHGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 156 aa, Artificial Sequence
  Description of Artificial Sequence Synthetic polypeptide
GILEECCFRSCDLALLETYCAGGGGSPLGLAGGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 52 - protein, 149 aa, Artificial Sequence
  Description of Artificial Sequence Synthetic polypeptide
AYRPSETLCGGELVDTLQFVCGDRGFAFGGGGSPLGLAGGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEI

SEQ ID NO: 53 - protein, 1461 aa, Artificial Sequence
  Description of Artificial Sequence Synthetic polypeptide
GGCATCGTGGAGGAGTGCTGCTTCAGAAGCTGCGATCTGGCTCTGCTGGAGACCTACTGCGCTGGAGGAGGCGGCAGCCCCCTGGGACTGGCTGGCGGAGGAGGAGGCAGCGAGGTGCAGCTGGTGGAGAGCGGAGGAGGACTGGTGCAGCCCGGCGGCTCTCTGAGACTGAGCTGTGCCGCCAGCGGCTTCGACTTTGCCGACTACGCCATGCACTGGGTGAGACAAGCCCCCGGCAAAGGACTGGAGTGGGTGAGCGCCATCACATGGAACGGCGGCCACACAGACTACGCCGATAGCGTGAAGGGAAGATTCACCATCTCTAGAGACAACAGCAAGAACACACTGTATCTGCAGATGAACTCTCTGAGAGCCGAGGACACCGCCGTGTACTACTGCGCCAAGGTGAGCTATCTGAGCACCGCCAGCTCTCTGGACTACTGGGGACAAGGCACACTGGTGACCGTGTCCTCCGCCAGCACCAAGGGCCCAAGCGTGTTTCCTCTGGCCCCCTCCTCCAAAAGCACATCCGGCGGAACAGCCGCCCTCGGATGTCTGGTCAAGGACTACTTCCCCGAACCCGTGACAGTCAGCTGGAATAGCGGCGCCCTCACAAGCGGCGTGCACACATTTCCCGCCGTGCTCCAGAGCAGCGGACTGTATTCTCTGTCCTCCGTGGTCACAGTCCCCAGCTCCTCCCTCGGCACCCAGACCTATATTTGCAACGTGAACCATAAGCCCTCCAACACCAAAGTCGACAAGAAGGTGGAGCCCAAGAGCTGCGATAAGACCCATACATGCCCCCCTTGTCCCGCCCCCCCTGCTGCTGGTCCCAGCGTGTTTTTATTTCCTCCTAAGCCCAAGGACACTTTAATGATCTCTCGTACCCCCGAGGTGACATGTGTGGTGGTGGACGTCTCCCATGAGGACCCCGAGGTGAAGTTCAATTGGTACGTCGACGGCGTGGAGGTGCACAATGCCAAGACCAAGCCTAGAGAGGAGCAGTACAACTCCACCTATAGGGTGGTGTCCGTGCTCACCGTGCTGCACCAAGATTGGCTGAACGGCAAGGAGTACAAGTGTAAGGTGTCCAACAAGGCTCTGCCCGCTCCCATCGAGAAGACCATCTCCAAGGCCAAAGGCCAGCCTAGAGAACCCCAAGTGTATACACTGCCCCCTAGCAGAGATGAACTGACCAAGAACCAAGTGTCTCTGACATGTCTGGTCAAGGGATTCTACCCCAGCGACATCGCCGTGGAGTGGGAGAGCAATGGCCAGCCCGAAAACAACTACAAGACCACCCCTCCCGTGCTCGACAGCGACGGATCCTTTCTCCTGTACAGCAAGCTGACCGTGGACAAAAGCAGATGGCAGCAAGGCAACGTCTTTAGCTGCAGCGTGCTGCACGAGGCTCTGCATAGCCACTACACACAGAAGTCTCTGTCTCTGTCCCCCGGCAAG

SEQ ID NO: 54 - protein, 774 aa, Artificial Sequence
  Description of Artificial Sequence Synthetic polypeptide
GCCTACAGACCCAGCGAGACACTGTGTGGCGGCGAGCTGGTGGACACACTGCAGTTCGTGTGCGGCGACAGAGGCTTCTACTTCGGCGGCGGAGGCAGCCCCCTGGGACTGGCTGGAGGCGGAGGCGGAAGCGAGATCGTGATGACCCAGAGCCCCGCCACACTGAGCGTGAGCCCCGGCGAGAGGGCCACACTGAGCTGCAGAGCCAGCCAAGGCATCAGAAACTATCTGGCTTGGTACCAGCAGAAGCCCGGCCAAGCCCCCAGACTGCTGATCTACGCCGCCAGCACACTGCAGAGCGGCATTCCCGCCAGATTCAGCGGAAGCGGCAGCGGAACCGAGTTCACACTGACCATCTCCTCTCTGCAGAGCGAGGACTTCGCCGTGTACTACTGCCAGAGGTACAACAGAGCCCCCTACACCTTCGGCCAAGGCACCAAGGTGGAGATCAAGCGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGT

SEQ ID NO: 55 - protein, 487 aa, Artificial Sequence
  Description of Artificial Sequence Synthetic polypeptide
GIVEECCFRSCDLALLETYCAGGGGSGPLGVRGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 56 - protein, 258 aa, Artificial Sequence
  Description of Artificial Sequence Synthetic polypeptide
AYRPSETLCGGELVDTLQFVCGDRGFYFGGGGSGPLGVRGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 487 aa, Artificial Sequence
  Description of Artificial Sequence Synthetic polypeptide
GIVEECCFRSCDLALLETYCAGGGGSPLGLARGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 58 - protein, 258 aa, Artificial Sequence
  Description of Artificial Sequence Synthetic polypeptide
AYRPSETLCGGELVDTLQFVCGDRGFYFGGGGSPLGLARGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 489 aa, Artificial Sequence
  Description of Artificial Sequence Synthetic polypeptide
GIVEECCFRSCDLALLETYCAGGGGSIPVSLRSGGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 60 - protein, 260 aa, Artificial Sequence
  Description of Artificial Sequence Synthetic polypeptide
AYRPSETLCGGELVDTLQFVCGDRGFYFGGGGSIPVSLRSGGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 489 aa, Artificial Sequence
  Description of Artificial Sequence Synthetic polypeptide
GIVEECCFRSCDLALLETYCAGGGGSGPLGMLSQGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 62 - protein, 260 aa, Artificial Sequence
  Description of Artificial Sequence Synthetic polypeptide
AYRPSETLCGGELVDTLQFVCGDRGFYFGGGGSGPLGMLSQGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 489 aa, Artificial Sequence
  Description of Artificial Sequence Synthetic polypeptide
GIVEECCFRSCDLALLETYCAGGGGSLSGRSDNHGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 64 - protein, 260 aa, Artificial Sequence
  Description of Artificial Sequence Synthetic polypeptide
AYRPSETLCGGELVDTLQFVCGDRGFYFGGGGSLSGRSDNHGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 450 aa, Artificial Sequence
  Description of Artificial Sequence Synthetic polypeptide
EVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 66 - protein, 214 aa, Artificial Sequence
  Description of Artificial Sequence Synthetic polypeptide
EIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPLLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 68 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 21 aa, Artificial Sequence
  Description of Artificial Sequence Synthetic peptide
GIAEECCFRSCDLALLETYCA

SEQ ID NO: 70 - protein, 21 aa, Artificial Sequence
  Description of Artificial Sequence Synthetic peptide
GILEECCFRSCDLALLETYCA

SEQ ID NO: 71 - protein, 28 aa, Artificial Sequence
  Description of Artificial Sequence Synthetic peptide
AYRPSETLCGGELVDTLQFVCGDRGFAF

SEQ ID NO: 72 - protein, 28 aa, Artificial Sequence
  Description of Artificial Sequence Synthetic peptide
AYRPSETLCGGELVDTLQFVCGDRGFLF

SEQ ID NO: 73 - protein, 487 aa, Artificial Sequence
  Description of Artificial Sequence Synthetic polypeptide
GILEECCFRSCDLALLETYCAGGGGSPLGLAGGGGGSEVQLVESGGGLVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 74 - protein, 258 aa, Artificial Sequence
  Description of Artificial Sequence Synthetic polypeptide
AYRPSETLCGGELVDTLQFVCGDRGFAFGGGGSPLGLAGGGGGSEIVMTQSPATLSVSPGERATLSCRASQGIRNYLAWYQQKPGQAPRLLIYAASTLQSGIPARFSGSGSGTEFTLTISSLQSEDFAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 487 aa, Artificial Sequence
  Description of Artificial Sequence Synthetic polypeptide
GILEECCFRSCDLALLETYCAGGGGSPLGLAGGGGGSEVQLVESGGVVVQPGGSLRLSCAASGFTFDDYAMHWVRQAPGKGLEWVSAITWNSGHIDYADSVKGRFTISRDNSKNSLYLQMNSLRTEDTALYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 76 - protein, 487 aa, Artificial Sequence
  Description of Artificial Sequence Synthetic polypeptide
GILEECCFRSCDLALLETYCAGGGGSPLGLAGGGGGSEVQLVESGGVVVQPGGSLRLSCAASGFDFADYAMHWVRQAPGKGLEWVSAITWNGGHTDYADSVKGRFTISRDNSKNSLYLQMNSLRTEDTALYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 77 - protein, 487 aa, Artificial Sequence
  Description of Artificial Sequence Synthetic polypeptide
GILEECCFRSCDLALLETYCAGGGGSPLGLAGGGGGSEVQLVESGGGLVQPGGSLRLSCAASGFTFDDYAMHWVRQAPGKGLEWVSAITWNSGHIDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 78 - protein, 487 aa, Artificial Sequence
  Description of Artificial Sequence Synthetic polypeptide
GILEECCFRSCDLALLETYCAGGGGSPLGLAGGGGGSQVQLVESGGGVVQPGGSLRLSCAASGFDFADYAMHWVRQAPGKGLEWVSAITWNGGHTDYADSVKGRFTISRDNSKNTLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 79 - protein, 258 aa, Artificial Sequence
  Description of Artificial Sequence Synthetic polypeptide
AYRPSETLCGGELVDTLQFVCGDRGFAFGGGGSPLGLAGGGGGSDIVMTQSPDSLAVSLGERATINCRASQGIRNYLAWYQQKPGQAPKLLIYAASTLQSGVPDRFSGSGSGTDFTLTISSLQAEDVAVYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 487 aa, Artificial Sequence
  Description of Artificial Sequence Synthetic polypeptide
GILEECCFRSCDLALLETYCAGGGGSPLGLAGGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 81 - protein, 258 aa, Artificial Sequence
  Description of Artificial Sequence Synthetic polypeptide
AYRPSETLCGGELVDTLQFVCGDRGFAFGGGGSPLGLAGGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVLHEALHSHYTQKSLSLSPGK

SEQ ID NO: 83 - protein, 17 aa, Artificial Sequence
  Description of Artificial Sequence Synthetic peptide
WEAAAREACCRECCARA

SEQ ID NO: 84 - protein, 5 aa, Artificial Sequence
  Description of Artificial Sequence Synthetic peptide
  See specification as filed for detailed description of substitutions and preferred embodiments
GSGGS

SEQ ID NO: 85 - protein, 4 aa, Artificial Sequence
  Description of Artificial Sequence Synthetic peptide
  See specification as filed for detailed description of substitutions and preferred embodiments
GGGS

SEQ ID NO: 86 - protein, 4 aa, Artificial Sequence
  Description of Artificial Sequence Synthetic peptide
GGSG

SEQ ID NO: 87 - protein, 5 aa, Artificial Sequence
  Description of Artificial Sequence Synthetic peptide
GGSGG

SEQ ID NO: 88 - protein, 5 aa, Artificial Sequence
  Description of Artificial Sequence Synthetic peptide
GGGGS

SEQ ID NO: 89 - protein, 5 aa, Artificial Sequence
  Description of Artificial Sequence Synthetic peptide
GSGSG

SEQ ID NO: 90 - protein, 5 aa, Artificial Sequence
  Description of Artificial Sequence Synthetic peptide
GSGGG

SEQ ID NO: 91 - protein, 5 aa, Artificial Sequence
  Description of Artificial Sequence Synthetic peptide
GGGSG

SEQ ID NO: 92 - protein, 5 aa, Artificial Sequence
  Description of Artificial Sequence Synthetic peptide
GSSSG

SEQ ID NO: 93 - protein, 5 aa, Artificial Sequence
  Description of Artificial Sequence Synthetic peptide
  See specification as filed for detailed description of substitutions and preferred embodiments
EAAAK

SEQ ID NO: 94 - protein, 5 aa, Artificial Sequence
  Description of Artificial Sequence Synthetic peptide
  MOD_RES (4)..(4): Any amino acid other than Pro
VPGXG

SEQ ID NO: 95 - protein, 9 aa, Artificial Sequence
  Description of Artificial Sequence Synthetic peptide
YPYDVPDYA

SEQ ID NO: 96 - protein, 8 aa, Artificial Sequence
  Description of Artificial Sequence Synthetic peptide
DYKDDDDK

SEQ ID NO: 97 - protein, 10 aa, Artificial Sequence
  Description of Artificial Sequence Synthetic peptide
EQKLISEEDL

SEQ ID NO: 98 - protein, 5 aa, Artificial Sequence
  Description of Artificial Sequence Synthetic 5xHis tag
HHHHH

SEQ ID NO: 99 - protein, 6 aa, Artificial Sequence
  Description of Artificial Sequence Synthetic 6xHis tag
HHHHHH

SEQ ID NO: 100 - protein, 8 aa, Artificial Sequence
  Description of Artificial Sequence Synthetic peptide
WSHPQFEK

SEQ ID NO: 101 - protein, 5 aa, Artificial Sequence
  Description of Artificial Sequence Synthetic peptide
RYIRS

SEQ ID NO: 102 - protein, 4 aa, Artificial Sequence
  Description of Artificial Sequence Synthetic peptide
FHHT